(19) United States
(12) Patent Application Publication
(10) Pub. No.: US 2024/0150322 A1
(43) Pub. Date: May 9, 2024

(54) CDK9 Inhibitors

(71) Applicant: Biolexis Therapeutics, Inc., Salt Lake City, UT (US)

(72) Inventors: Hariprasad VANKAYALAPATI, Sandy, UT (US); Zhaoliang LI, Salt Lake City, UT (US); Kyle MEDLEY, American Fork, UT (US); Dongqing YAN, Salt Lake City, UT (US); David J. BEARSS, Alpine, UT (US)

(21) Appl. No.: 18/296,935

(22) Filed: Apr. 6, 2023

Related U.S. Application Data
(60) Provisional application No. 63/329,139, filed on Apr. 8, 2022.

(51) Int. Cl. C07D 403/04; A61P 35/00; C07D 471/04

(52) U.S. Cl. CPC C07D 403/04; A61P 35/00; C07D 471/04

(57) ABSTRACT

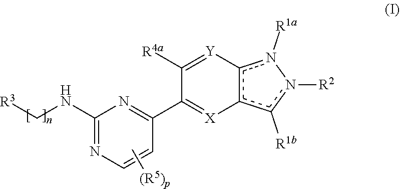

or a salt (e.g., pharmaceutically acceptable salt) or stereoisomer thereof, wherein X, Y, R^{1a}, R^{1b}, R², R³, R^{4s}, R⁵, p, and n are as defined herein. Use of the compounds as a component of a pharmaceutical compositions and methods for their use are also provided.

### STATEMENT REGARDING FEDERALLY SPONSORED RESEARCH OR DEVELOPMENT

Portions of the work described herein were made with government support under grant number W81XWH2110170 awarded by the United States Department of Defense. The government has certain rights in the invention.

### BACKGROUND

### Technical Field

The present disclosure generally relates to compounds that inhibit protein kinase activity such as cyclin-dependent protein kinases (CDKs), and to compositions and methods to treat cancers and other conditions that are associated with CDKs.

### Description of the Related Art

Cyclin-dependent kinases (CDKs) are serine-threonine kinases that function to coordinate multiple cellular functions and phosphorylate substrates essential for progression through the cell cycle. Activity of specific CDKs at specific times is essential for both initiation and coordinated progress through the cell cycle. For example, CDK7, CDK8, and CDK9 play a role in regulating transcription to further influence cell proliferation and survival by driving the expression of numerous target genes. The relevance of CDKs to cancer growth and survival has garnered widespread interest in the generation of CDK inhibitors.

A great number of CDK9 inhibitors have been widely used in tumor cells to the rapid induction of apoptosis. However, a complex toxicity profile associated with activity beyond CDK9 has hampered development.

Therefore, there is a need for the rational design of specific and selective CDK9 inhibitors for the treatment of cancer and other conditions that are mediated and/or associated. The present disclosure fulfills these needs and offers other related advantages.

### BRIEF SUMMARY

In brief, the present disclosure provides CDK9 inhibitor compounds, including stereoisomers or salts (e.g., pharmaceutically acceptable salts) thereof, which can be used alone or in combination with a pharmaceutically acceptable carrier. Methods for use of CDK9 inhibitor compounds for treatment of various diseases or conditions, such as bladder cancer, prostate cancer, and leukemia are also provided.

In one embodiment, compounds having the following Structure (I) are provided:

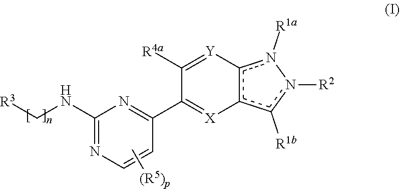

or a salt (e.g., pharmaceutically acceptable salt) or stereoisomer thereof, wherein X, Y, R^{1a}, R^{1b}, R², R³, R^{4s}, R⁵, p, and n are as defined herein. Use of the compounds as a component of a pharmaceutical compositions and methods for their use are also provided. Pharmaceutical compositions comprising one or more of the foregoing compounds of Structure (I) and a therapeutic agent are also provided.

In other embodiments, the present disclosure provides a method for administering a therapeutic agent to a patient in need thereof, the method comprising preparing a composition comprising the compound of Structure (I) and a therapeutic agent and delivering the composition to the patient.

These and other aspects of the disclosure will be apparent upon reference to the following detailed description.

### BRIEF DESCRIPTION OF THE DRAWINGS

In the figures, identical reference numbers identify similar elements. The sizes and relative positions of elements in the figures are not necessarily drawn to scale and some of these elements are enlarged and positioned to improve figure legibility. Further, the particular shapes of the elements as drawn are not intended to convey any information regarding the actual shape of the particular elements and have been solely selected for ease of recognition in the figures.

FIG. 1 is a chart illustrating cellular antiproliferative efficacy of compound I-2 on six prostate cancer cell lines over various concentrations.

FIG. 2 is a chart illustrating cellular antiproliferative efficacy of a benchmark compound AZD-4573 on six prostate cancer cell lines over various concentrations.

FIG. 3 is a bar graph illustrating cellular antiproliferative efficacy of compound I-2 on 22RV-1 colony.

FIG. 4 is a bar graph illustrating cellular antiproliferative efficacy of compound I-2 on LASCPC-1 colony.

FIG. 5 is a series of images from LASCPC-01 cells.

FIG. 6 is a series of charts showing Annexin V/7-AAD staining for LASCPC-01.

FIG. 7 is a series of charts showing Annexin V/7-AAD staining for 22Rev1.

FIG. 8 is an image of Western Blot analysis of N-MYC.

FIG. 9 is an image of Western Blot analysis of N-MYC and C-MYC.

FIG. 10 is a bar graph showing RT-PCR analysis for 22Rv1 and LASCPC-01.

FIG. 11 is a series of images of staining for LASCPC-01 cells.

FIG. 12 is an image of Western Blot from 22Rv1 with compound I-2 and AZD-4573.

FIG. 13 is an image of Western Blot from LASCPC-01 with compound I-2 and AZD-4573.

FIG. 14 is a chart showing 22Rv1 average tumor volume comparison.

FIG. 15 is a chart showing LASCPC-01 average tumor volume comparison.

FIG. 16 shows relative body weights of groups of treated and untreated mice FIG. 17 illustrates 22Rv1 mean tumor volume over a 21-day study for different doses of compound I-2.

FIG. 18 shows tumor growth inhibition plotted against dose and tumor weights.

FIG. 19 shows relative body weights of groups of treated and untreated mice.

FIG. 20 illustrates LASCPC-01 mean tumor volume over a 21-day study for different doses of compound I-2.

FIG. 21 shows tumor growth inhibition plotted against dose and tumor weights.

FIG. 22 depicts the effect of compound I-2 (50 mg/kg, p.o., daily) changed to (30 mg/kg, p.o. bi-weekly) on tumor volume in C4-2 xenograft in male NOD·SCID mice. Statistical analysis was performed using GraphPad Prism software version 5.0., two-way ANOVA with Bonferroni's post-hoc test; All the values are expressed as Mean±SEM, 7-8 mice per group, *P<0.05, ***P<0.001 vs. Vehicle control

FIG. 23 shows the effect of compound I-2 (50 mg/kg, p.o., daily) changed to (30 mg/kg, p.o., bi-weekly) on tumor growth inhibition in C4-2 xenograft in male NOD·SCID mice.

FIG. 24 illustrates the effect of compound I-2 (50 mg/kg, p.o., daily) changed to (30 mg/kg, p.o. bi-weekly) on tumor weight in C4-2 xenograft in male NOD·SCID mice. Statistical analysis was performed using GraphPad Prism software version 5.0., t-test; All the values are expressed as Mean±SEM, 8 mice per group, *P<0.05 vs. Vehicle control.

### DETAILED DESCRIPTION

In the following description, certain specific details are set forth in order to provide a thorough understanding of various embodiments of the disclosure. However, one skilled in the art will understand that the disclosure may be practiced without these details. As used herein, the following terms have the meanings ascribed to them unless specified otherwise.

Unless the context requires otherwise, throughout the present specification and claims, the word “comprise” and variations thereof, such as, “comprises” and “comprising” are to be construed in an open and inclusive sense, that is, as “including, but not limited to”.

Reference throughout this specification to “one embodiment” or “an embodiment” means that a particular feature, structure, or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the features, structures, or characteristics may be combined in any suitable manner in one or more embodiments.

Unless defined otherwise, all technical and scientific terms used herein have the same meaning as is commonly understood by one of skill in the art to which this disclosure belongs. As used in the specification and claims, the singular form “a”, “an” and “the” include plural references unless the context clearly dictates otherwise.

“Hydroxy” or “hydroxyl” refers to an —OH radical.

“Amino” refers to an —NH₂ radical.

“Cyano” refers to a —CN radical.

“Alkyl” refers to a saturated straight or branched hydrocarbon chain radical consisting solely of carbon and hydrogen atoms having from one to six carbon atoms (C₁-C₆ alkyl), which is attached to the rest of the molecule by a single bond. Hydrocarbon chain radicals include, for example, methyl, ethyl, n-propyl, 1 methylethyl (iso-propyl), n-butyl, n-pentyl, 1,1 dimethylethyl (t-butyl), iso-pentyl, n-hexyl, and the like. Unless stated otherwise specifically in the specification, an alkyl group is optionally substituted.

“Cycloalkyl” refers to a saturated cyclic hydrocarbon radical having from three to eight carbon atoms (C₃-C₈ cycloalkyl) attached to the rest of the molecule by a single bond. Saturated cyclic hydrocarbon radicals include, for example, cyclopropyl, cyclobutyl, cyclopentyl or cyclohexyl. Unless otherwise stated specifically in the specification, a cycloalkyl group is optionally substituted.

“Bridged cycloalkyl” refers to a cycloalkyl as defined above that also includes an alkylene bridge between at least two carbon atoms of the cycle. Bridged cycloalkyl radicals include, for example, bicyclo[2.1.1]hexanyl, bicyclo[2.2.1]heptanyl, bicyclo[3.1.1]heptanyl, (1R, 5S)-bicyclo[3.2.1]octanyl, and bicyclo[2.2.2]octanyl. Unless otherwise stated specifically in the specification, a bridged cycloalkyl group is optionally substituted.

“Halo” refers to fluoro, chloro, bromo, or iodo. Halo belongs to group 17 of the periodic table.

“Alkoxy” refers to a radical of the formula —ORₐ where Rₐ is an alkyl radical as defined above containing one to twelve carbon atoms (C₁-C₁₂ alkoxy), one to eight carbon atoms (C₁-C₈ alkoxy) or one to six carbon atoms (C₁-C₆ alkoxy), or any value within these ranges. Unless stated otherwise specifically in the specification, an alkoxy group is optionally substituted.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group is optionally substituted.

“Haloalkoxy” refers to a radical having the following formula: —O-haloalkyl, wherein haloalkyl is as defined above. Unless otherwise stated specifically in the specification, a haloalkoxy group is optionally substituted.

“Hydroxylalkyl” or “hydroxyalkyl” refers to an alkyl radical, as defined above that is substituted by one or more hydroxyl radical. The hydroxyalkyl radical is joined at the main chain through the alkyl carbon atom. Unless stated otherwise specifically in the specification, a hydroxyalkyl group is optionally substituted.

“Aryl” refers to an all-carbon monocyclic or fused-ring polycyclic (i.e., rings which share adjacent pairs of carbon atoms) groups of 6 to 12 carbon atoms having a completely conjugated pi-electron system. Examples, without limitation, of aryl groups are phenyl, naphthyl and anthracenyl. The aryl group may be substituted or unsubstituted. When substituted, the aryl group is substituted with one or more substituents as this term is defined below, more preferably one, two or three, even more preferably one or two substituents independently selected from the group consisting of alkyl (wherein the alkyl may be optionally substituted with one or two substituents), haloalkyl, halo, hydroxy, alkoxy, mercapto, alkylthio, cyano, acyl, nitro, phenoxy, heteroaryl, heteroaryloxy, haloalkyl, haloalkoxy, carboxy, alkoxycarbonyl, amino, alkylamino dialkylamino, aryl, heteroaryl, carbocycle or heterocycle (wherein the aryl, heteroaryl, carbocycle or heterocycle may be optionally substituted).

“Heterocyclyl” or “heterocyclic ring” refers to a stable 3- to 18-membered non-aromatic ring radical which consists of two to twelve carbon atoms and from one to six heteroatoms selected from the group consisting of nitrogen, oxygen, and sulfur. Unless stated otherwise specifically in the specification, the heterocyclyl radical may be a monocyclic, bicyclic, tricyclic or tetracyclic ring system, which may include fused or bridged ring systems; and the nitrogen, carbon or sulfur atoms in the heterocyclyl radical may be optionally oxidized; the nitrogen atom may be optionally quaternized; and the heterocyclyl radical may be partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group may be optionally substituted.

“N-heterocyclyl” refers to a heterocyclyl radical as defined above, wherein at least one heteroatom or heteroatoms are nitrogen. Examples of N-heterocyclyl radicals include azetidinyl, pyrrolidinyl, piperidinyl, piperazinyl, morpholinyl, and the like. Unless stated otherwise specifically in the specification, an N-heterocyclyl group may be optionally substituted.

“Heteroaryl” refers to a 5- to 18-membered, for example 5- to 6-membered, ring system radical comprising one to thirteen ring carbon atoms, one to six ring heteroatoms selected from the group consisting of nitrogen, oxygen and sulfur, and at least one aromatic ring. Heteroaryl radicals may be a monocyclic, bicyclic, tricyclic or tetracyclic ring system, which may include fused or bridged ring systems; and the nitrogen, carbon or sulfur atoms in the heteroaryl radical may be optionally oxidized; the nitrogen atom may be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl group is optionally substituted.

The term “substituted” used herein means any of the above groups (e.g., alkyl, cycloalkyl or heterocyclyl) wherein at least one hydrogen atom is replaced by a bond to a non-hydrogen atoms such as, but not limited to: a halogen atom such as F, Cl, Br, and I; oxo groups (═O); hydroxyl groups (—OH); alkoxy groups (—OR^{a}, where R^{a} is C₁-C₁₂ alkyl or cycloalkyl); carboxyl groups (—OC(═O)R^{a} or —C(═O)OR^{a}, where R^{a} is H, C₁-C₁₂ alkyl or cycloalkyl); amine groups (—NR^{a}R^{b}, where R^{a} and R^{b} are each independently H, C₁-C₁₂ alkyl or cycloalkyl); C₁-C₁₂ alkyl groups; and cycloalkyl groups. In some embodiments the substituent is a C₁-C₁₂ alkyl group. In other embodiments, the substituent is a cycloalkyl group. In other embodiments, the substituent is a halo group, such as fluoro. In other embodiments, the substituent is a oxo group. In other embodiments, the substituent is a hydroxyl group. In other embodiments, the substituent is an alkoxy group. In other embodiments, the substituent is a carboxyl group. In other embodiments, the substituent is an amine group.

“Optional” or “optionally” (e.g., optionally substituted) means that the subsequently described event of circumstances may or may not occur, and that the description includes instances where said event or circumstance occurs and instances in which it does not. For example, “optionally substituted alkyl” means that the alkyl radical may or may not be substituted and that the description includes both substituted alkyl radicals and alkyl radicals having no substitution.

“Prodrug” is meant to indicate a compound that may be converted under physiological conditions or by solvolysis to a biologically active compound of the disclosure. Thus, the term “prodrug” refers to a metabolic precursor of a compound of the disclosure that is pharmaceutically acceptable. A prodrug may be inactive when administered to a subject in need thereof but is converted in vivo to an active compound of the disclosure. Prodrugs are typically rapidly transformed in vivo to yield the parent compound of the disclosure, for example, by hydrolysis in blood. The prodrug compound often offers advantages of solubility, tissue compatibility or delayed release in a mammalian organism (see, Bundgard, H., Design of Prodrugs (1985), pp. 7-9, 21-24 (Elsevier, Amsterdam)). A discussion of prodrugs is provided in Higuchi, T., et al., A.C.S. Symposium Series, Vol. 14, and in Bioreversible Carriers in Drug Design, Ed. Edward B. Roche, American Pharmaceutical Association and Pergamon Press, 1987.

The term “prodrug” is also meant to include any covalently bonded carriers, which release the active compound of the disclosure in vivo when such prodrug is administered to a mammalian subject. Prodrugs of a compound of the disclosure may be prepared by modifying functional groups present in the compound of the disclosure in such a way that the modifications are cleaved, either in routine manipulation or in vivo, to the parent compound of the disclosure. Prodrugs include compounds of the disclosure wherein a hydroxy, amino or mercapto group is bonded to any group that, when the prodrug of the compound of the disclosure is administered to a mammalian subject, cleaves to form a free hydroxy, free amino or free mercapto group, respectively. Examples of prodrugs include, but are not limited to, acetate, formate and benzoate derivatives of alcohol or amide derivatives of amine functional groups in the compounds of the disclosure and the like.

The embodiments disclosed herein is also meant to encompass all pharmaceutically acceptable compounds of the compound of Structure (I) being isotopically labelled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²p, ³⁵S, ¹⁸F, ³⁶Cl, ¹²³I, and ¹²⁵I, respectively. These radiolabeled compounds could be useful to help determine or measure the effectiveness of the compounds, by characterizing, for example, the site or mode of action, or binding affinity to pharmacologically important site of action. Certain isotopically labelled compounds of Structure (I), for example, those incorporating a radioactive isotope, are useful in drug and/or substrate tissue distribution studies. The radioactive isotopes tritium, i.e., ³H, and carbon-14, i.e., ¹⁴C, are particularly useful for this purpose in view of their ease of incorporation and ready means of detection.

Substitution with heavier isotopes such as deuterium, i.e., ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy. Isotopically labeled compounds of Structure (I) can generally be prepared by conventional techniques known to those skilled in the art or by processes analogous to those described in the Preparations and Examples as set out below using an appropriate isotopically labeled reagent in place of the non-labeled reagent previously employed.

The embodiments disclosed herein is also meant to encompass the in vivo metabolic products of the disclosed compounds. Such products may result from, for example, the oxidation, reduction, hydrolysis, amidation, esterification, and the like of the administered compound, primarily due to enzymatic processes. Accordingly, the disclosure includes compounds produced by a process comprising administering a compound of this disclosure to a mammal for a period of time sufficient to yield a metabolic product thereof. Such products are typically identified by administering a radiolabeled compound of the disclosure in a detectable dose to an animal, such as rat, mouse, guinea pig, monkey, or to human, allowing sufficient time for metabolism to occur, and isolating its conversion products from the urine, blood or other biological samples.

“Stable compound” and “stable structure” are meant to indicate a compound that is sufficiently robust to survive isolation to a useful degree of purity from a reaction mixture, and formulation into an efficacious therapeutic agent.

“Mammal” includes humans and both domestic animals such as laboratory animals and household pets (e.g., cats, dogs, swine, cattle, sheep, goats, horses, rabbits), and non-domestic animals such as wildlife and the like.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid (TFA), undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

A “pharmaceutical composition” refers to a formulation of a compound of the disclosure and a medium generally accepted in the art for the delivery of the biologically active compound to mammals, e.g., humans. Such a medium includes all pharmaceutically acceptable carriers, diluents or excipients therefor.

“Effective amount” or “therapeutically effective amount” refers to that amount of a compound of the disclosure which, when administered to a mammal, preferably a human, is sufficient to effect treatment in the mammal, preferably a human. The amount of a lipid nanoparticle of the disclosure which constitutes a “therapeutically effective amount” will vary depending on the compound, the condition and its severity, the manner of administration, and the age of the mammal to be treated, but can be determined routinely by one of ordinary skill in the art having regard to his own knowledge and to this disclosure.

“Protein kinase-mediated condition” or “disease,” as used herein, refers to any disease or other deleterious condition in which a protein kinase is known to play a role and that are alleviated by treatment with a protein kinase inhibitor. In certain embodiments, the cancer is a cancer of colon, breast, stomach, prostate, pancreas, or ovarian tissue.

“Treating” or “treatment” as used herein covers the treatment of the disease or condition of interest in a mammal, preferably a human, having the disease or condition of interest, and includes:
(i) preventing the disease or condition from occurring in a mammal, in particular, when such mammal is predisposed to the condition but has not yet been diagnosed as having it;
(ii) inhibiting the disease or condition, i.e., arresting its development;
(iii) relieving the disease or condition, i.e., causing regression of the disease or condition; or
(iv) relieving the symptoms resulting from the disease or condition, i.e., relieving pain without addressing the underlying disease or condition. As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

The compounds of the disclosure or their salts (e.g., pharmaceutically acceptable salts) may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present disclosure is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present disclosure contemplates various stereoisomers and mixtures thereof and includes “enantiomers”, which refers to two stereoisomers whose molecules are nonsuperimposeable mirror images of one another. The present disclosure also contemplates “diastereomers”, which refers to non-mirror image of non-identical stereoisomers. Diastereomers occur when two or more stereoisomers of a compound have different configurations at one or more of the equivalent stereocenters and are not mirror images of each other.

A “tautomer” refers to a proton shift from one atom of a molecule to another atom of the same molecule. The present disclosure includes tautomers of any said compounds.

### Compounds

In an aspect, the present disclosure provides CDK9 inhibitor compounds, including stereoisomers or salts (e.g., pharmaceutically acceptable salts) thereof, which can be used alone or in combination with a pharmaceutically acceptable carrier. Methods for use of CDK9 inhibitor compounds for treatment of various diseases or conditions, such as bladder cancer, prostate cancer, and leukemia are also provided.

One embodiment provides a compound having the following Structure (I):

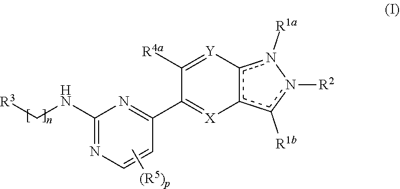

as a stereoisomer or salt thereof, wherein:
[symbol] represents a double or single bond such that all valences are satisfied and a heteroaryl ring is formed;
X is N or CR^{4b};
Y is N or CR^{4c};
R^{1a} is absent or C₁-C₆ alkyl;
R^{1b} is hydrogen, halo, hydroxy, C₁-C₆ alkyl, C₃-C₈ cycloalkyl, C₁-C₆ haloalkyl, C₁-C₆ alkoxy, or C₁-C₆ hydroxyalkyl;
R² is hydrogen or optionally substituted: C₁-C₆ alkyl, C₃-C₈ cycloalkyl, C₁-C₆ haloalkyl, or C₁-C₆ hydroxyalkyl;
R³ is C₃-C₅ cycloalkyl, C₆-C₈ bridged cycloalkyl, or 3-10 membered heterocyclyl, optionally substituted with hydroxyl, amino, cyano, halo, C₁-C₆ alkyl, C₃-C₈ cycloalkyl, C₁-C₆ haloalkyl, C₁-C₆ alkoxy, C₁-C₆ haloalkoxy, or —N(R^{3c})R^{3a}, or
R³ is C₃-C₈ cycloalkyl optionally substituted with hydroxyl, cyano, halo, C₁-C₆ alkyl, C₃-C₈ cycloalkyl, C₁-C₆ haloalkyl, C₁-C₆ alkoxy, C₁-C₆ haloalkoxy, or —N(R^{3c})R^{3b};
R^{3a} is optionally substituted 3-10 membered heterocyclyl or optionally substituted 3-10 membered heteroaryl;
R^{3b} is optionally substituted 3-10 membered N-heterocyclyl or optionally substituted 3-10 membered heteroaryl;
R^{3c} is hydrogen or optionally substituted C₁-C₆ alkyl;
R^{4a}, R^{4b}, and R^{4c} are each independently hydrogen, halo or optionally substituted: C₁-C₆ alkyl, C₃-C₈ cycloalkyl, C₁-C₆ haloalkyl, C₁-C₆ alkoxy, or C₁-C₆ haloalkoxy;
each occurrence of R⁵ is independently halo or optionally substituted C₁-C₆ haloalkyl;
n is 0, 1, 2, 3, or 4; and
p is 0, 1, or 2.

One embodiment provides a compound having the following Structure (I′):

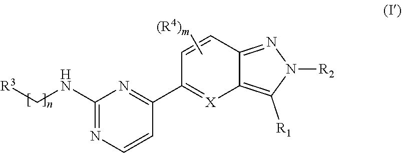

as a stereoisomer or salt (e.g., pharmaceutically acceptable salt) thereof, wherein:
X is N or CH;
R¹ is hydrogen, halo, hydroxy or optionally substituted: C₁-C₆ alkyl, C₃-C₈ cycloalkyl, C₁-C₆ haloalkyl, C₁-C₆ alkoxy, or C₁-C₆ hydroxyalkyl;
R² is hydrogen or optionally substituted: C₁-C₆ alkyl, C₃-C₈ cycloalkyl, C₁-C₆ haloalkyl, or C₁-C₆ hydroxyalkyl;
R³ is C₃-C₅ cycloalkyl or 3-10 membered heterocyclyl, optionally substituted with hydroxyl, amino, cyano, halo, C₁-C₆ alkyl, C₃-C₈ cycloalkyl, C₁-C₆ haloalkyl, C₁-C₆alkoxy, C₁-C₆ haloalkoxy, or —N(R^{3c})R^{3a}, or
R³ is C₃-C₈ cycloalkyl optionally substituted with hydroxyl, cyano, halo, C₁-C₆ alkyl, C₃-C₈ cycloalkyl, C₁-C₆ haloalkyl, C₁-C₆ alkoxy, C₁-C₆ haloalkoxy, or —N(R^{3c})R^{3b};
R^{3a} is optionally substituted 3-10 membered heterocyclyl or optionally substituted 3-10 membered heteroaryl;
R^{3b} is optionally substituted 3-10 membered N-heterocyclyl or optionally substituted 3-10 membered heteroaryl;
R^{3c} is hydrogen or optionally substituted C₁-C₆ alkyl;
R⁴ is halo or optionally substituted: C₁-C₆ alkyl, C₃-C₈ cycloalkyl, C₁-C₆ haloalkyl, C₁-C₆ alkoxy, or C₁-C₆ haloalkoxy;
m is 0, 1, or 2; and
n is 0, 1, 2, 3, or 4.

In some embodiments, the compound has one of the following structures (Ia) or (Ib):

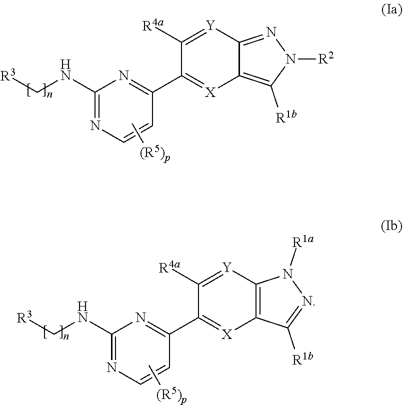

In certain embodiments, R^{1a} is absent. In some embodiments, R^{1a} is hydrogen. In some embodiments, R^{1a} is C₁-C₆ alkyl. In certain embodiments, R^{1a} is methyl.

In some embodiments, R¹ is hydrogen, C₁-C₆ alkyl, or C₃-C₈ cycloalkyl. In certain embodiments, R¹ is hydrogen, cyclopropyl, cyclobutyl, cyclopentyl, cyclohexyl, isopropyl, methyl, or ethyl. In some embodiments, R¹ is hydrogen, cyclopropyl, cyclobutyl, isopropyl, or methyl. In certain embodiments, R¹ is unsubstituted.

In some embodiments, R^{1b} is hydrogen, C₁-C₆ alkyl, or C₃-C₈ cycloalkyl. In certain embodiments, R^{1b} is hydrogen, cyclopropyl, cyclobutyl, cyclopentyl, cyclohexyl, isopropyl, methyl, or ethyl. In some embodiments, R^{1b} is hydrogen, cyclopropyl, cyclobutyl, isopropyl, or methyl. In certain embodiments, R^{1b} is unsubstituted.

In some embodiments, R² is hydrogen or C₁-C₆ alkyl. In more specific embodiments, R² is C₁-C₆ alkyl. In some embodiments, R² is —CH₃. In certain embodiments, R² is methyl, ethyl, iso-propyl or n-propyl. In some embodiments, R² is hydrogen. In certain embodiments, R² is unsubstituted.

In some embodiments, at least one of R¹ or R² is hydrogen. In certain embodiments, both of R¹ and R² are hydrogen.

In some embodiments, at least one of R^{1b} or R² is hydrogen. In certain embodiments, both of R^{1b} and R² are hydrogen.

In some embodiments, n is 0 or 1. In certain embodiments, n is 0. In some embodiments, n is 2, 3, or 4.

In some embodiments, R³ is C₃-C₅ cycloalkyl or 3-10 membered heterocyclyl optionally substituted with hydroxyl, amino, cyano, halo, C₁-C₆ alkyl, C₃-C₈ cycloalkyl, C₁-C₆ haloalkyl, C₁-C₆ alkoxy, C₁-C₆ haloalkoxy, or —N(R^{3c})R^{3a}.

In some embodiments, R³ is C₃-C₅ cycloalkyl, C₆-C₇ bridged cycloalkyl, or 3-10 membered heterocyclyl optionally substituted with hydroxyl, amino, cyano, halo, C₁-C₆ alkyl, C₃-C₈ cycloalkyl, C₁-C₆ haloalkyl, C₁-C₆ alkoxy, C₁-C₆ haloalkoxy, or —N(R^{3c})R^{3a}.

In certain embodiments, R³ is C₅ cycloalkyl optionally substituted with hydroxyl, amino, cyano, halo, C₁-C₆ alkyl, C₃-C₈ cycloalkyl, C₁-C₆ haloalkyl, C₁-C₆ alkoxy, C₁-C₆ haloalkoxy, or —N(R^{3c})R^{3a}. In some embodiments, R³ is C₅ cycloalkyl optionally substituted with hydroxyl or amino. In some embodiments, R³ is C₅ cycloalkyl optionally substituted with amino. In some embodiments, R³ is C₃-C₈ cycloalkyl optionally substituted with hydroxyl, cyano, halo, C₁-C₆ alkyl, C₃-C₈ cycloalkyl, C₁-C₆ haloalkyl, C₁-C₆ alkoxy, C₁-C₆ haloalkoxy, or —N(R^{3c})R^{3b}. In some embodiments, R³ is 5-6 membered heterocyclyl. In some embodiments, R³ is substituted. In more specific embodiments, R³ is unsubstituted.

In certain embodiments, R³ has one of the following structures:

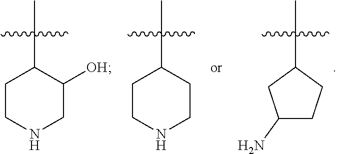

In certain embodiments, R³ has one of the following structures:

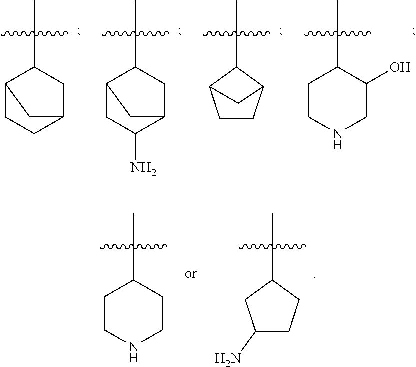

In some embodiments, R³ has one of the following structures:

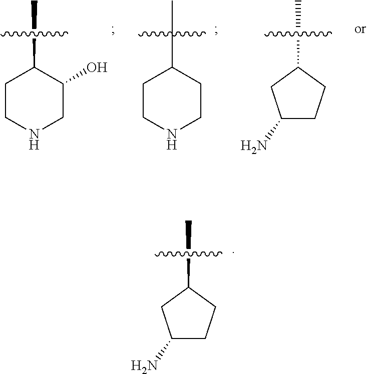

In some embodiments, R³ has one of the following structures:

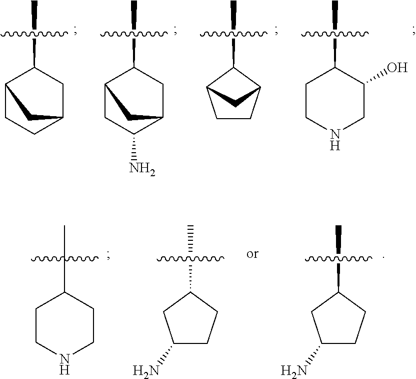

In some embodiments, R³ has the following structure:

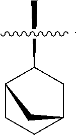

In certain embodiments, R³ has the following structure:

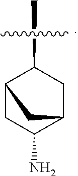

In some embodiments, R³ has the following structure:

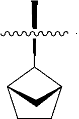

In certain embodiments, R³ has the following structure:

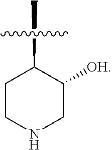

In some embodiments, R³ has the following structure:

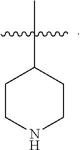 [SMILES: *C1CCNCC1]

In certain embodiments, R³ has the following structure:

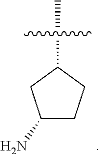

In some embodiments, R³ has the following structure:

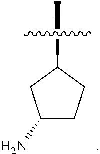

In some embodiments, m is 1 or 2. In certain embodiments, each occurrence of R⁴ is independently halo. In some embodiments, each occurrence of R⁴ is independently fluoro, chloro, or bromo. In some more specific embodiments, each occurrence of R⁴ is fluoro. In some embodiments, m is 0.

In some embodiments, R^{4a} is hydrogen. In some embodiments, R^{4a} is halo (e.g., chloro, fluoro, or bromo).

In some embodiments, X is N. In some embodiments, X is CR^{4b} and R^{4b} is hydrogen or halo. In some embodiments, X is CR^{4b} and R^{4b} is hydrogen or fluoro. In certain embodiments, X is CF. In some embodiments, X is CH.

In some embodiments, Y is N. In some embodiments, Y is CR^{4b} and R^{4b} is hydrogen or halo. In some embodiments, Y is CR^{4b} and R^{4b} is hydrogen or fluoro. In certain embodiments, Y is CF. In some embodiments, Y is CH.

In some embodiments, p is 1. In some embodiments, p is 2. In certain embodiments, each occurrence of R⁵ is halo (e.g., chloro, fluoro, or bromo). In some embodiments, p is 0. In some embodiments, p is 1 and R⁵ is chloro.

In some embodiments, the compound is a free base form. In certain embodiments, the compound is a pharmaceutically acceptable salt. In some embodiments, the compound is a trifluoroacetic acid salt. In some embodiments, the compound is a trifluoroacetic acid salt, a hydrochloric acid salt, or a formic acid salt. In certain embodiments, the compound is a tautomer.

In various different embodiments, the compound has one of the structures set forth in Table 1 below (or a stereoisomer or salt thereof).

TABLE 1
Representative compounds of Structure (I)

| | | | Mol. | IC₅₀ |
|---|---|---|---|---|
| No. | Compound | Name | Wt. | (μM) |
| I-1^{†} | 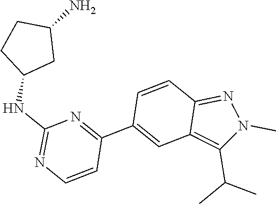 [SMILES: CC(C)c1c2cc(-c3ccnc(N[C@@H]4CC[C@H](N)C4)n3)ccc2nn1C] | (1S,3R)-[3-[4-(3- isopropyl-2- methylindazol-5- yl)pyrimidin-2- yl]aminocyclopentan- 1-yl]-1,3-diamine | 578.52 | ++++ |
| I-2^{†} | 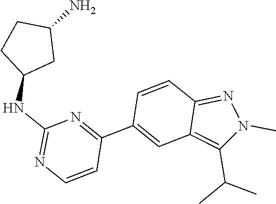 [SMILES: CC(C)c1c2cc(-c3ccnc(N[C@H]4CC[C@H](N)C4)n3)ccc2nn1C] | (1S,3S)-[3-[4-(3- isopropyl-2- methylindazol-5- yl)pyrimidin-2- yl]aminocyclopentan- 1-yl]-1,3-diamine | 578.52 | ++++ |
| I-3^{†} | 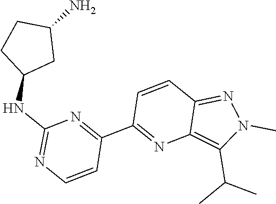 [SMILES: CC(C)c1c2nc(-c3ccnc(N[C@H]4CC[C@H](N)C4)n3)ccc2nn1C] | (1S,3S)-[3-[4-[3- isopropyl-2- methylpyrazolo[4,3- b]pyridin-5- yl]pyrimidin-2- yl]aminocyclopentan- 1-yl]-1,3-diamine | 579.50 | + |
| I-4^{†} | 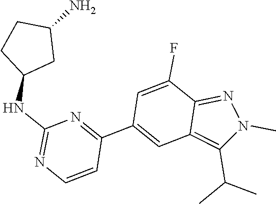 [SMILES: CC(C)c1c2cc(-c3ccnc(N[C@H]4CC[C@H](N)C4)n3)cc(F)c2nn1C] | (1S,3S)-[3-[4-(7- fluoro-3-isopropyl-2- methylindazol-5- yl)pyrimidin-2- yl]aminocyclopentan- 1-yl]-1,3-diamine | 482.48 | ++++ |
| I-5 | 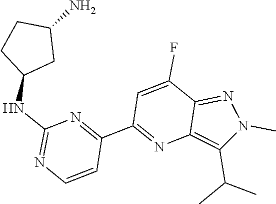 [SMILES: CC(C)c1c2nc(-c3ccnc(N[C@H]4CC[C@H](N)C4)n3)cc(F)c2nn1C] | (1S,3S)-[3-[4-(7- fluoro-3-isopropyl-2- methylpyrazolo[4,3- b]pyridin-5- yl)pyrimidin-2- yl]aminocyclopentan- 1-yl]-1,3-diamine | 369.45 | — |
| I-6^{‡} | 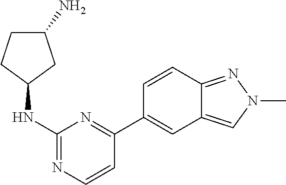 [SMILES: Cn1cc2cc(-c3ccnc(N[C@H]4CC[C@H](N)C4)n3)ccc2n1] | (1S,3S)-N¹-(4-(2- methyl-2H-indazol-5- yl)pyrimidin-2- yl)cyclopentane-1,3- diamine | 381.30 | ++++ |
| I-7^{‡} | 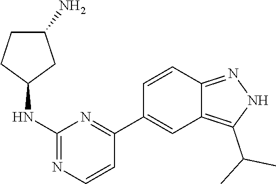 [SMILES: CC(C)c1[nH]nc2ccc(-c3ccnc(N[C@H]4CC[C@H](N)C4)n3)cc12] | (1S,3S)-N¹-(4-(3- isopropyl-2H-indazol- 5-yl)pyrimidin-2- yl)cyclopentane-1,3- diamine | 409.36 | ++++ |
| I-8^{‡} | 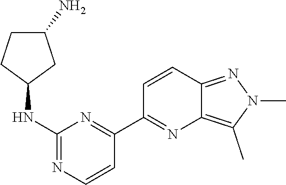 [SMILES: Cc1c2nc(-c3ccnc(N[C@H]4CC[C@H](N)C4)n3)ccc2nn1C] | (1S,3S)-N¹-(4-(2,3- dimethyl-2H-indazol- 5-yl)pyrimidin-2- yl)cyclopentane-1,3- diamine | 323.40 | — |
| I-9^{‡} | 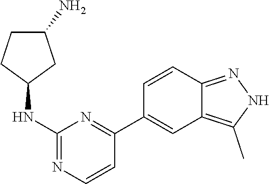 [SMILES: Cc1[nH]nc2ccc(-c3ccnc(N[C@H]4CC[C@H](N)C4)n3)cc12] | (1S,3S)-N¹-(4-(3- methyl-2H-indazol-5- yl)pyrimidin-2- yl)cyclopentane-1,3- diamine | 308.39 | +++ |
| I-10^{‡} | 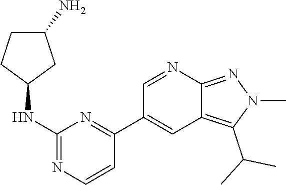 [SMILES: CC(C)c1c2cc(-c3ccnc(N[C@H]4CC[C@H](N)C4)n3)cnc2nn1C] | (1S,3S)-N¹-(4-(3- isopropyl-2-methyl- 2H-pyrazolo[3,4- b]pyridin-5- yl)pyrimidin-2- yl)cyclopentane-1,3- diamine | 424.37 | ++++ |
| I-11^{‡} | 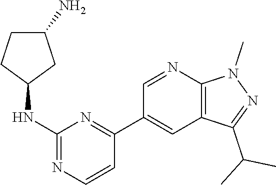 [SMILES: CC(C)c1nn(C)c2ncc(-c3ccnc(N[C@H]4CC[C@H](N)C4)n3)cc12] | (1S,3S)-N¹-(4-(3- isopropyl-1-methyl- 1H-pyrazolo[3,4- b]pyridin-5- yl)pyrimidin-2- yl)cyclopentane-1,3- diamine | 424.37 | + |
| I-12 | 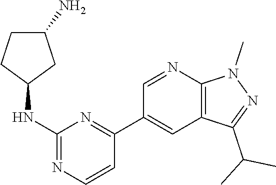 [SMILES: CC(C)c1nn(C)c2ncc(-c3ccnc(N[C@H]4CC[C@H](N)C4)n3)cc12] | (1S,3S)-N¹-(4-(4- fluoro-3-isopropyl-2- methyl-2H- pyrazolo[3,4- b]pyridin-5- yl)pyrimidin-2- yl)cyclopentane-1,3- diamine | 369.45 | — |
| I-13 | 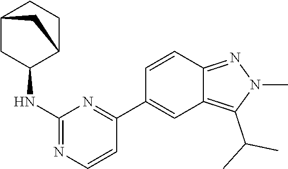 [SMILES: CC(C)c1c2cc(-c3ccnc(N[C@H]4C[C@@H]5CC[C@H]4C5)n3)ccc2nn1C] | N-((1R,2S,4R)- bicyclo[2.2.1]heptan- 2-yl)-4-(3-isopropyl-2- methyl-2H-indazol-5- yl)pyrimidin-2-amine | 361.49 | ++++ |
| I-14^{†} | 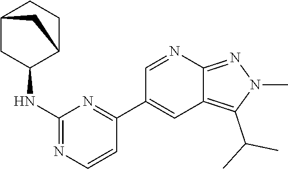 [SMILES: CC(C)c1c2cc(-c3ccnc(N[C@H]4C[C@@H]5CC[C@H]4C5)n3)cnc2nn1C] | N-((1R,2S,4R)- bicyclo[2.2.1]heptan- 2-yl)-4-(3-isopropyl-2- methyl-2H- pyrazolo[3,4- b]pyridin-5- yl)pyrimidin-2-amine | 362.48 | ++++ |
| I-15 | 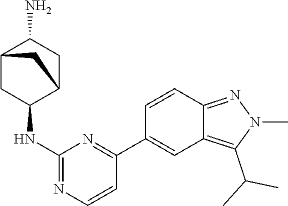 [SMILES: CC(C)c1c2cc(-c3ccnc(N[C@H]4C[C@H]5C[C@@H]4C[C@H]5N)n3)ccc2nn1C] | (1S,2S,4R,5R)-N²-(4- (3-isopropyl-2-methyl- 2H-indazol-5- yl)pyrimidin-2- yl)bicyclo[2.2.1] heptane-2,5-diamine | 376.51 | — |
| I-16 | 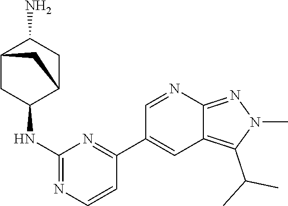 [SMILES: CC(C)c1c2cc(-c3ccnc(N[C@H]4C[C@H]5C[C@@H]4C[C@H]5N)n3)cnc2nn1C] | (1S,2S,4R,5R)-N²-(4- (3-isopropyl-2-methyl- 2H-pyrazolo[3,4- b]pyridin-5- yl)pyrimidin-2- yl)bicyclo[2.2.1] heptane-2,5-diamine | 377.50 | — |
| I-17 | 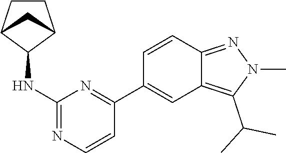 [SMILES: CC(C)c1c2cc(-c3ccnc(N[C@H]4[C@@H]5CC[C@H]4C5)n3)ccc2nn1C] | N-((1R,4R)- bicyclo[2.1.1]hexan-5- yl)-4-(3-isopropyl-2- methyl-2H-indazol-5- yl)pyrimidin-2-amine | 347.47 | — |
| I-18 | 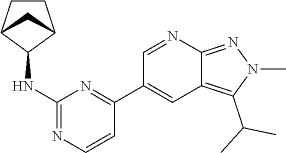 [SMILES: CC(C)c1c2cc(-c3ccnc(N[C@H]4[C@@H]5CC[C@H]4C5)n3)cnc2nn1C] | N-((1R,4R)- bicyclo[2.1.1]hexan-5- yl)-4-(3-isopropyl-2- methyl-2H- pyrazolo[3,4- b]pyridin-5- yl)pyrimidin-2-amine | 348.45 | — |

^{†}Also obtained as a trifluoroacetic acid salt
^{‡}Also obtained as a hydrochloric acid salt
# Also obtained as a formic acid salt
IC₅₀ values:
++++ represents a value below 0.05 μM
+++ represents a value between 0.05 and 0.10 μM
++ represents a value between 0.10 and 1.00 μM
+ represents a value greater than 1 μM

It is understood that any embodiment of the compounds of Structure (I) as set forth above, and any specific substituent and/or variable in the compound of Structure (I) as set forth above may be independently combined with other embodiments and/or substituents and/or variables of compounds of Structure (I) to form embodiments of the disclosure not specifically set forth above. In addition, in the event that a list of substituents and/or variables is listed for any particular R group or variables n or m in a particular embodiment and/or claim, it is understood that each individual substituent and/or variable may be deleted from the particular embodiment and/or claim and that the remaining list of substituents and/or variables will be considered to be within the scope of the disclosure. It is understood that in the present description, combinations of substituents and/or variables of the depicted formulae are permissible only if such contributions result in stable compounds.

### Pharmaceutical Compositions

Other embodiments are directed to pharmaceutical compositions. The pharmaceutical composition comprises anyone (or more) of the foregoing compounds and a pharmaceutically acceptable carrier. In some embodiments, the pharmaceutical composition is formulated for oral administration. In other embodiments, the pharmaceutical composition is formulated for injection. In still more embodiments, the pharmaceutical compositions comprise a compound as disclosed herein and an additional therapeutic agent (e.g., anticancer agent). Non-limiting examples of such therapeutic agents are described herein below.

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, a compound as described herein is administered in a local rather than systemic manner, for example, via injection of the compound directly into an organ, often in a depot preparation or sustained release formulation. In specific embodiments, long-acting formulations are administered by implantation (for example subcutaneously or intramuscularly) or by intramuscular injection. Furthermore, in other embodiments, the compound is delivered in a targeted drug delivery system, for example, in a liposome coated with and organ specific antibody. In such embodiments, the liposomes are targeted to and taken up selectively by the organ. In yet other embodiments, the compound as described herein is provided in the form of a rapid release formulation, in the form of an extended-release formulation, or in the form of an intermediate release formulation. In yet other embodiments, the compound described herein is administered topically.

In treatment methods according to embodiments of the disclosure, an effective amount of at least one compound of Structure (I) is administered to a subject suffering from or diagnosed as having such a disease, disorder, or medical condition. Effective amounts or doses may be ascertained by methods such as modeling, dose escalation studies or clinical trials, e.g., the mode or route of administration or drug delivery, the pharmacokinetics of the agent, the severity and course of the disease, disorder, or condition, the subject's previous or ongoing therapy, the subject's health status and response to drugs, and the judgment of the treating physician.

The compounds according to the disclosure are effective over a wide dosage range. For example, in the treatment of adult humans, dosages from 10 to 5000 mg, from 100 to 5000 mg, from 1000 mg to 4000 mg per day, and from 1000 to 3000 mg per day are examples of dosages that are used in some embodiments. The exact dosage will depend upon the route of administration, the form in which the compound is administered, the subject to be treated, the body weight of the subject to be treated, and the preference and experience of the attending physician.

In some embodiments, compounds of the disclosure are administered in a single dose. Typically, such administration will be by injection, e.g., intravenous injection, in order to introduce the agent quickly. However, other routes are used as appropriate. A single dose of a compound of the disclosure may also be used for treatment of an acute condition.

In some embodiments, compounds of the disclosure are administered in multiple doses. In some embodiments, dosing is about once, twice, three times, four times, five times, six times, or more than six times per day. In other embodiments, dosing is about once a month, once every two weeks, once a week, or once every other day. In another embodiment compounds of the disclosure and another agent (e.g., anti-cancer agent) are administered together about once per day to about 6 times per day. In another embodiment the administration of compounds of the disclosure and an agent continues for less than about 7 days. In yet another embodiment the administration continues for more than about 6, 10, 14, 28 days, two months, six months, or one year. In some cases, continuous dosing is achieved and maintained as long as necessary.

Administration of compounds of the disclosure may continue as long as necessary. In some embodiments, compounds of the disclosure are administered for more than 1, 2, 3, 4, 5, 6, 7, 14, or 28 days. In some embodiments, compounds of the disclosure are administered for less than 28, 14, 7, 6, 5, 4, 3, 2, or 1 day. In some embodiments, compounds of the disclosure are administered chronically on an ongoing basis, e.g., for the treatment of chronic effects.

In some embodiments, the compounds of the disclosure are administered in individual dosage forms. It is known in the art that due to intersubject variability in compound pharmacokinetics, individualization of dosing regimen is necessary for optimal therapy.

In some embodiments, the compounds described herein are formulated into pharmaceutical compositions. In specific embodiments, pharmaceutical compositions are formulated in a conventional manner using one or more physiologically acceptable carriers comprising excipients and auxiliaries which facilitate processing of the disclosed compounds into preparations which can be used pharmaceutically. Proper formulation is dependent upon the route of administration chosen. Any pharmaceutically acceptable techniques, carriers, and excipients are used as suitable to formulate the pharmaceutical compositions described herein: Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

Provided herein are pharmaceutical compositions comprising one or more compounds of Structure (I), and a pharmaceutically acceptable carrier.

Provided herein are pharmaceutical compositions comprising one or more compounds selected from compounds of Structure (I) and pharmaceutically acceptable diluent(s), excipient(s), and carrier(s). In certain embodiments, the compounds described are administered as pharmaceutical compositions in which one or more compounds selected from compounds of Structure (I) are mixed with other active ingredients, as in combination therapy. Encompassed herein are all combinations of actives set forth in the combination therapies section below and throughout this disclosure. In specific embodiments, the pharmaceutical compositions include one or more compounds of Structure (I).

A pharmaceutical composition, as used herein, refers to a mixture of one or more compounds selected from compounds of Structure (I) with other chemical components, such as carriers, stabilizers, diluents, dispersing agents, suspending agents, thickening agents, and/or excipients. In certain embodiments, the pharmaceutical composition facilitates administration of the compound to an organism. In some embodiments, therapeutically effective amounts of one or more compounds selected from compounds of Structure (I) provided herein are administered in a pharmaceutical composition to a mammal having a disease, disorder or medical condition to be treated. In specific embodiments, the mammal is a human. In certain embodiments, therapeutically effective amounts vary depending on the severity of the disease, the age and relative health of the subject, the potency of the compound used and other factors. The compounds described herein are used singly or in combination with one or more therapeutic agents as components of mixtures.

In one embodiment, one or more compounds selected from compounds of Structure (I) are formulated in aqueous solutions. In specific embodiments, the aqueous solution is selected from, by way of example only, a physiologically compatible buffer, such as Hank's solution, Ringer's solution, or physiological saline buffer. In other embodiments, one or more compounds selected from compounds of Structure (I) are formulated for transmucosal administration. In specific embodiments, transmucosal formulations include penetrants that are appropriate to the barrier to be permeated. In still other embodiments wherein the compounds described herein are formulated for other parenteral injections, appropriate formulations include aqueous or non-aqueous solutions. In specific embodiments, such solutions include physiologically compatible buffers and/or excipients.

In another embodiment, compounds described herein are formulated for oral administration. Compounds described herein are formulated by combining the active compounds with, e.g., pharmaceutically acceptable carriers or excipients. In various embodiments, the compounds described herein are formulated in oral dosage forms that include, by way of example only, tablets, powders, pills, dragees, capsules, liquids, gels, syrups, elixirs, slurries, suspensions and the like.

In certain embodiments, pharmaceutical preparations for oral use are obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. In specific embodiments, disintegrating agents are optionally added. Disintegrating agents include, by way of example only, cross linked croscarmellose sodium, polyvinylpyrrolidone, agar, or alginic acid or a salt thereof such as sodium alginate.

In one embodiment, dosage forms, such as dragee cores and tablets, are provided with one or more suitable coating. In specific embodiments, concentrated sugar solutions are used for coating the dosage form. The sugar solutions, optionally contain additional components, such as by way of example only, gum arabic, talc, polyvinylpyrrolidone, carbopol gel, polyethylene glycol, and/or titanium dioxide, lacquer solutions, and suitable organic solvents or solvent mixtures. Dyestuffs and/or pigments are also optionally added to the coatings for identification purposes. Additionally, the dyestuffs and/or pigments are optionally utilized to characterize different combinations of active compound doses.

In certain embodiments, therapeutically effective amounts of at least one of the compounds described herein are formulated into other oral dosage forms. Oral dosage forms include push fit capsules made of gelatin, as well as soft, sealed capsules made of gelatin and a plasticizer, such as glycerol or sorbitol. In specific embodiments, push fit capsules contain the active ingredients in admixture with one or more filler. Fillers include, by way of example only, lactose, binders such as starches, and/or lubricants such as talc or magnesium stearate and, optionally, stabilizers. In other embodiments, soft capsules, contain one or more active compound that is dissolved or suspended in a suitable liquid. Suitable liquids include, by way of example only, one or more fatty oil, liquid paraffin, or liquid polyethylene glycol. In addition, stabilizers are optionally added.

In still other embodiments, the compounds described herein are formulated for parental injection, including formulations suitable for bolus injection or continuous infusion. In specific embodiments, formulations for injection are presented in unit dosage form (e.g., in ampoules) or in multi dose containers. Preservatives are, optionally, added to the injection formulations. In still other embodiments, the pharmaceutical compositions are formulated in a form suitable for parenteral injection as sterile suspensions, solutions or emulsions in oily or aqueous vehicles. Parenteral injection formulations optionally contain formulatory agents such as suspending, stabilizing and/or dispersing agents. In specific embodiments, pharmaceutical formulations for parenteral administration include aqueous solutions of the active compounds in water soluble form. In additional embodiments, suspensions of one or more compounds selected from compounds of Structure (I) are prepared as appropriate oily injection suspensions. Suitable lipophilic solvents or vehicles for use in the pharmaceutical compositions described herein include, by way of example only, fatty oils such as sesame oil, or synthetic fatty acid esters, such as ethyl oleate or triglycerides, or liposomes. In certain specific embodiments, aqueous injection suspensions contain substances which increase the viscosity of the suspension, such as sodium carboxymethyl cellulose, sorbitol, or dextran. Optionally, the suspension contains suitable stabilizers or agents which increase the solubility of the compounds to allow for the preparation of highly concentrated solutions. Alternatively, in other embodiments, the active ingredient is in powder form for constitution with a suitable vehicle, e.g., sterile pyrogen-free water, before use.

Pharmaceutical compositions include at least one pharmaceutically acceptable carrier, diluent or excipient, and one or more compounds selected from compounds of Structure (I), described herein as an active ingredient. The active ingredient is in free-acid or free-base form, or in a pharmaceutically acceptable salt form. In addition, the methods and pharmaceutical compositions described herein include the use of N-oxides, crystalline forms (also known as polymorphs), as well as active metabolites of these compounds having the same type of activity. All tautomers of the compounds described herein are included within the scope of the compounds presented herein. Additionally, the compounds described herein encompass unsolvated as well as solvated forms with pharmaceutically acceptable solvents such as water, ethanol, and the like. The solvated forms of the compounds presented herein are also considered to be disclosed herein. In addition, the pharmaceutical compositions optionally include other medicinal or pharmaceutical agents, carriers, adjuvants, such as preserving, stabilizing, wetting or emulsifying agents, solution promoters, salts for regulating the osmotic pressure, buffers, and/or other therapeutically valuable substances.

Methods for the preparation of compositions comprising the compounds described herein include formulating the compounds with one or more inert, pharmaceutically acceptable excipients or carriers to form a solid, semi-solid or liquid. Solid compositions include, but are not limited to, powders, tablets, dispersible granules, capsules, cachets, and suppositories. Liquid compositions include solutions in which a compound is dissolved, emulsions comprising a compound, or a solution containing liposomes, micelles, or nanoparticles comprising a compound as disclosed herein. Semi-solid compositions include, but are not limited to, gels, suspensions and creams. The form of the pharmaceutical compositions described herein include liquid solutions or suspensions, solid forms suitable for solution or suspension in a liquid prior to use, or as emulsions. These compositions also optionally contain minor amounts of nontoxic, auxiliary substances, such as wetting or emulsifying agents, pH buffering agents, and so forth.

In some embodiments, pharmaceutical compositions comprising one or more compounds selected from compounds of Structure (I) illustratively takes the form of a liquid where the agents are present in solution, in suspension or both. Typically when the composition is administered as a suspension, a first portion of the agent is present in solution and a second portion of the agent is present in particulate form, in suspension in a liquid matrix. In some embodiments, a liquid composition includes a gel formulation. In other embodiments, the liquid composition is aqueous.

In certain embodiments, aqueous suspensions contain one or more polymers as suspending agents. Polymers include water-soluble polymers such as cellulosic polymers, e.g., hydroxypropyl methylcellulose, and water-insoluble polymers such as cross-linked carboxyl-containing polymers. Certain pharmaceutical compositions described herein comprise a mucoadhesive polymer, selected for example from carboxymethylcellulose, carbomer (acrylic acid polymer), poly(methylmethacrylate), polyacrylamide, polycarbophil, acrylic acid/butyl acrylate copolymer, sodium alginate and dextran.

Pharmaceutical compositions also, optionally, include solubilizing agents to aid in the solubility of one or more compounds selected from compounds of Structure (I). The term “solubilizing agent” generally includes agents that result in formation of a micellar solution or a true solution of the agent. Certain acceptable nonionic surfactants, for example polysorbate 80, are useful as solubilizing agents, as can ophthalmically acceptable glycols, polyglycols, e.g., polyethylene glycol 400, and glycol ethers.

Furthermore, pharmaceutical compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Compositions also, optionally, include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other pharmaceutical compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

Compositions may include one or more surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

Compositions may include one or more antioxidants to enhance chemical stability where required. Suitable antioxidants include, by way of example only, ascorbic acid and sodium metabisulfite.

In certain embodiments, aqueous suspension compositions are packaged in single-dose non-reclosable containers. Alternatively, multiple-dose reclosable containers are used, in which case it is typical to include a preservative in the composition.

In alternative embodiments, other delivery systems for hydrophobic pharmaceutical compounds are employed. Liposomes and emulsions are examples of delivery vehicles or carriers useful herein. In certain embodiments, organic solvents such as N-methylpyrrolidone are also employed. In additional embodiments, the compounds described herein are delivered using a sustained release system, such as semipermeable matrices of solid hydrophobic polymers containing the therapeutic agent. Various sustained release materials are useful herein. In some embodiments, sustained release capsules release the compounds for a few weeks up to over 100 days. Depending on the chemical nature and the biological stability of the therapeutic reagent, additional strategies for protein stabilization are employed.

In certain embodiments, the formulations described herein comprise one or more antioxidants, metal chelating agents, thiol containing compounds and/or other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

In some embodiments, the concentration of one or more compounds selected from compounds of Structure (I) provided in the pharmaceutical compositions of the present disclosure is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v.

In some embodiments, the concentration of one or more compounds selected from compounds of Structure (I) provided in the pharmaceutical compositions of the present disclosure is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v.

In some embodiments, the amount the one or more compounds selected from compounds of Structure (I) provided in the pharmaceutical compositions of the present disclosure is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of the one or more compounds selected from compounds of Structure (I) provided in the pharmaceutical compositions of the present disclosure is in the range of 0.0001-10 g, 0.0005-9 g, 0.001-8 g, 0.005-7 g, 0.01-6 g, 0.05-5 g, 0.1-4 g, 0.5-4 g, or 1-3 g.

Packaging materials for use in packaging pharmaceutical compositions described herein include those found in, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. For example, the container(s) includes one or more compounds described herein, optionally in a composition or in combination with another agent as disclosed herein. The container(s) optionally have a sterile access port (for example the container is an intravenous solution bag or a vial having a stopper pierceable by a hypodermic injection needle). Such kits optionally comprise a compound with an identifying description or label or instructions relating to its use in the methods described herein.

For example, a kit typically includes one or more additional containers, each with one or more of various materials (such as reagents, optionally in concentrated form, and/or devices) desirable from a commercial and user standpoint for use of a compound described herein. Non-limiting examples of such materials include, but not limited to, buffers, diluents, filters, needles, syringes; carrier, package, container, vial and/or tube labels listing contents and/or instructions for use, and package inserts with instructions for use. A set of instructions will also typically be included. A label is optionally on or associated with the container. For example, a label is on a container when letters, numbers or other characters forming the label are attached, molded or etched into the container itself, a label is associated with a container when it is present within a receptacle or carrier that also holds the container, e.g., as a package insert. In addition, a label is used to indicate that the contents are to be used for a specific therapeutic application. In addition, the label indicates directions for use of the contents, such as in the methods described herein. In certain embodiments, the pharmaceutical compositions are presented in a pack or dispenser device which contains one or more unit dosage forms containing a compound provided herein. The pack for example contains metal or plastic foil, such as a blister pack. Or, the pack or dispenser device is accompanied by instructions for administration. Or, the pack or dispenser is accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In some embodiments, compositions containing a compound provided herein formulated in a compatible pharmaceutical carrier are prepared, placed in an appropriate container, and labeled for treatment of an indicated condition.

One embodiment provides a method of treating neuroendocrine prostate cancer (NEPC). Some embodiments provide a method for treating overexpression of N-MYC or MCL-1. Some embodiments provide a method of treating metastatic castration-resistant prostate cancers, or CRPCs. In some embodiments the disorder or disease includes treatment of a tumor with drug resistance. In some embodiments, the NEPC arises from prostate adenocarcinoma, androgen deprivation therapy (ADT), or abnormal expression and activation of various kinases. Some embodiments provide a method for treating hematological tumors or solid tumors.

In some embodiments, the method includes inhibiting or silencing pCDK9, pSer2, P-TEFb, MYC oncogene transcriptional activity, or suppression of active super-enhancer complex.

In some embodiments, the method includes promoting prostate cancer cell death and overcoming drug resistance (e.g., chemotherapeutic resistance and other current targeted therapeutics).

In certain embodiments, the method includes inhibiting CDK9, N-MYC, C-MYC, and its associated super-enhancer genes expression profile. In some embodiments, the method includes increasing the (median) survival time to greater than 12, 13, 13.5, 13.6, 13.7, 14, 18, 20 or 36 months. In some embodiments, the method further comprises administering a compound of Structure (I) in combination with another chemotherapeutic (e.g., docetaxel, abiraterone, enzalutamide).

In some embodiments, the method includes inhibiting CDK9 as a treatment for prostate tumors. In some embodiments, the method includes increasing prostate cancer cell death and overcoming the resistance due to chemo- and current targeted therapeutics. In some embodiments, the method includes inhibiting the CDK9-cyclin T complex, which phosphorylates the negative elongation factor (NELF) complex, DRB-sensitivity inducing factor (DSIF), and the Ser2 of the CTD of RNAPII, thereby affecting the removal of elongation blocks.

In some embodiments, the method includes reversibly binding to and inhibiting CDK9. In some embodiments, the method includes treating malignant prostate cancer cells, especially those of the CRPC, mCRPC, NEPC and treatment-resistant subtype origin, while also demonstrating no toxicity to normal prostate cells. In some embodiments, the method includes modulating N-MYC, C-MYC and MCL-1 transcription (e.g., in NEPC cells such as 22RV1, LASCPC-01, C4-2 and C4-2B from prostate cancer patients). In certain embodiments, the method includes orally administering compound of Structure (I). In some embodiments, the method includes globally modulating transcription, silencing, and inhibiting CDK9. In some embodiments, the method includes dually inhibiting CDK9, MYC, and associated super-enhancer genes. In some embodiments, the method includes inhibiting tumor growth.

In certain embodiments, the method includes binding or targeting residues within the CDK9 ATP binding site, for example, gate keeper Phe103, hinge residues Asp104, Phe105, Cys106 and DFG loop (167, 168, 169 residues) including back pocket K48 and sugar binding pockets residues Glu107, His108 and Asp109.

As mentioned above, the compounds and compositions of the disclosure will find utility in a broad range of diseases and conditions mediated by protein kinases, including diseases and conditions mediated by kinase. Such diseases may include by way of example and not limitation, cancers such as lung cancer, NSCLC (non small cell lung cancer), oat-cell cancer, bone cancer, pancreatic cancer, skin cancer, dermatofibrosarcoma protuberans, cancer of the head and neck, cutaneous or intraocular melanoma, uterine cancer, ovarian cancer, colo-rectal cancer, cancer of the anal region, stomach cancer, colon cancer, breast cancer, gynecologic tumors (e.g., uterine sarcomas, carcinoma of the fallopian tubes, carcinoma of the endometrium, carcinoma of the cervix, carcinoma of the vagina or carcinoma of the vulva), Hodgkin's Disease, hepatocellular cancer, cancer of the esophagus, cancer of the small intestine, cancer of the endocrine system (e.g., cancer of the thyroid, pancreas, parathyroid or adrenal glands), sarcomas of soft tissues, cancer of the urethra, cancer of the penis, prostate cancer (particularly hormone-refractory), chronic or acute leukemia, solid tumors of childhood, hypereosinophilia, lymphocytic lymphomas, cancer of the bladder, cancer of the kidney or ureter (e.g., renal cell carcinoma, carcinoma of the renal pelvis), pediatric malignancy, neoplasms of the central nervous system (e.g., primary CNS lymphoma, spinal axis tumors, medulloblastoma, brain stem gliomas or pituitary adenomas), Barrett's esophagus (pre-malignant syndrome), neoplastic cutaneous disease, psoriasis, mycoses fungoides, and benign prostatic hypertrophy, diabetes related diseases such as diabetic retinopathy, retinal ischemia, and retinal neovascularization, hepatic cirrhosis, angiogenesis, cardiovascular disease such as atherosclerosis, immunological disease such as autoimmune disease and renal disease.

In some embodiments, a pharmaceutical composition has a compound described above and a pharmaceutically acceptable carrier including, for example, any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

In some embodiments, a method treating a disease or disorder, the method includes administering an effective amount of the compound or the pharmaceutical composition described herein to a subject in need thereof.

In some embodiments, the disease or disorder is a kinase-expressing cancer. In some specific embodiments, the cancer is bladder cancer. In some other specific embodiments, the cancer is prostate cancer. In some other specific embodiments, the cancer is a hematological malignancy such as acute myeloid leukemia. In some other specific embodiments, the disease or disorder is an autoimmune or inflammatory disease.

### Preparation of Compounds

Preparation methods for the above compounds and compositions are described herein below and/or known in the art. It will be appreciated by those skilled in the art that in the process described herein the functional groups of intermediate compounds may need to be protected by suitable protecting groups. Such functional groups include hydroxy, amino, mercapto and carboxylic acid. Suitable protecting groups for hydroxy include trialkylsilyl or diarylalkylsilyl (for example, t-butyldimethylsilyl, t-butyldiphenylsilyl or trimethylsilyl), tetrahydropyranyl, benzyl, and the like. Suitable protecting groups for amino, amidino and guanidino include t-butoxycarbonyl, benzyloxycarbonyl, and the like. Suitable protecting groups for mercapto include —C(O)—R″ (where R″ is alkyl, aryl or arylalkyl), p-methoxybenzyl, trityl and the like. Suitable protecting groups for carboxylic acid include alkyl, aryl or arylalkyl esters. Protecting groups may be added or removed in accordance with standard techniques, which are known to one skilled in the art and as described herein. The use of protecting groups is described in detail in Green, T. W. and P. G. M. Wutz, Protective Groups in Organic Synthesis (1999), 3rd Ed., Wiley. As one of skill in the art would appreciate, the protecting group may also be a polymer resin such as a Wang resin, Rink resin or a 2-chlorotrityl-chloride resin.

It will also be appreciated by those skilled in the art, although such protected derivatives of compounds of this disclosure may not possess pharmacological activity as such, they may be administered to a mammal and thereafter metabolized in the body to form compounds of the disclosure which are pharmacologically active. Such derivatives may therefore be described as “prodrugs”. All prodrugs of compounds of this disclosure are included within the scope of the disclosure.

Furthermore, all compounds of the disclosure which exist in free base or acid form can be converted to their pharmaceutically acceptable salts by treatment with the appropriate inorganic or organic base or acid by methods known to one skilled in the art. Salts of the compounds of the disclosure can be converted to their free base or acid form by standard techniques.

The following Reaction Scheme illustrates methods to make compounds of this disclosure, i.e., compounds of Structure (I):

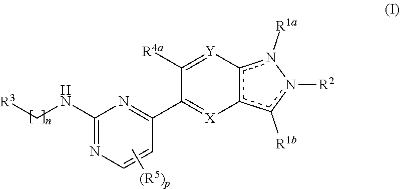

or a salt (e.g., pharmaceutically acceptable salt) or stereoisomer thereof, wherein X, Y, R^{1a}, R^{1b}, R², R³, R^{4s}, R⁵, p, and n are as defined herein. It is understood that one skilled in the art may be able to make these compounds by similar methods or by combining other methods known to one skilled in the art. It is also understood that one skilled in the art would be able to make, in a similar manner as described below, other compounds of Structure (I) not specifically illustrated below by using the appropriate starting components and modifying the parameters of the synthesis as needed. In general, starting components may be obtained from sources such as Sigma Aldrich, Lancaster Synthesis, Inc., Maybridge, Matrix Scientific, TCI, and Fluorochem USA, etc. or synthesized according to sources known to those skilled in the art (see, for example, Advanced Organic Chemistry: Reactions, Mechanisms, and Structure, 5th edition (Wiley, December 2000)) or prepared as described in this disclosure.

The following examples are provided for purpose of illustration and not limitation.

### Abbreviations

° C. (degree Celsius); ¹H NMR (proton Nuclear Magnetic Resonance); DCM (dichloromethane); DMSO (dimethylsulfoxide); eq (equivalent); EtOAc (ethyl acetate); g (gram); h (hour); MeOH (methanol); mg (milligram); min (minute); mL (milliliter); mmol (millimole); TFA (trifluoroacetic acid); THF (tetrahydrofuran); TLC (Thin Layer Chromatography); LDA (lithium diisopropylamide); AcOH (acetic acid); mCPBA (3-chloroperbenzoic acid).

### Example 1

### Synthesis of Compound I-1

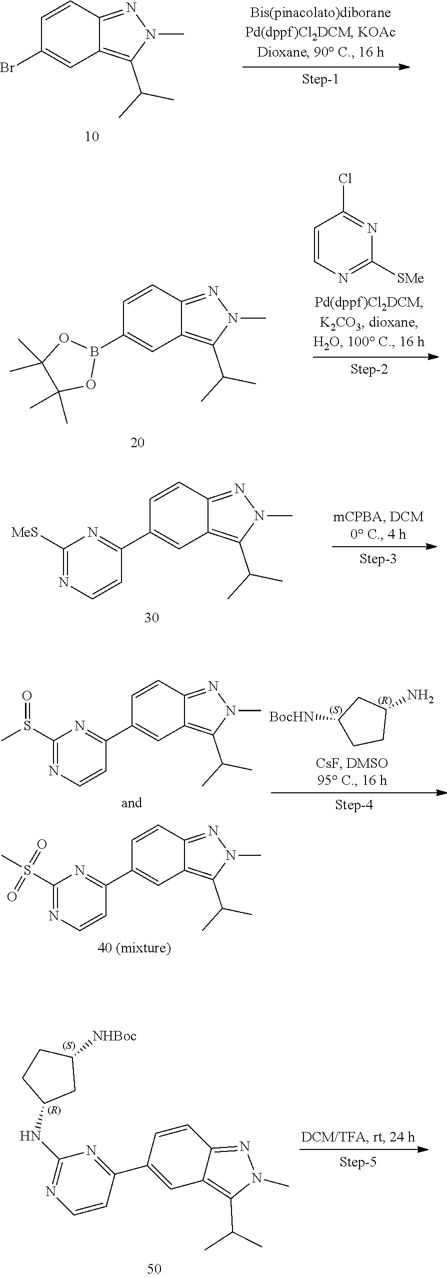

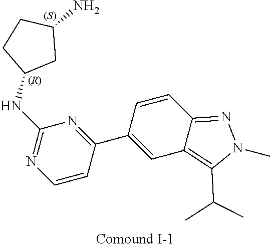

### Synthesis of 3-isopropyl-2-methyl-5-(4,4,5,5-tetramethyl-1,3,2-dioxaborolan-2-yl)indazole (20)

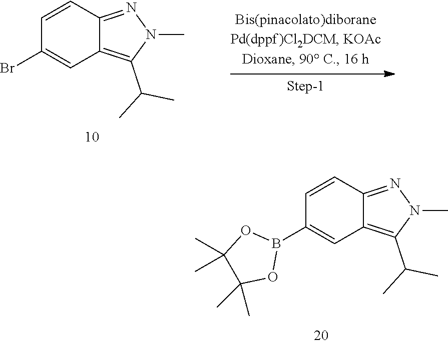

To a stirred solution of 5-bromo-2methyl-3-(propan-2-yl-2H-indazole) (10) (127 mg, 0.50 mmol, 1.0 eq) and bis(pinacolato)diborane (152 mg, 0.60 mmol, 1.2 eq) in 1,4-dioxane (8 mL) in a microwave vial was added potassium acetate (119 mg, 1.25 mmol, 2.5 eq), Pd(dppf)Cl₂·DCM (20 mg, 0.025 mmol, 0.05 eq). Then, the vial was sealed with the cap and degassed for 5 min with argon. The reaction mixture was stirred at 90° C. for 16 h. After completion of reaction by TLC, reaction mixture was diluted with water and ethyl acetate, resulting slurry was filtered through a pad of Celite® (i.e., diatomaceous earth). The organic layer was separated, washed with brine solution, dried over sodium sulfate and concentrated to provide crude product, 3-isopropyl-2-methyl-5-(4,4,5,5-tetramethyl-1,3,2-dioxaborolan-2-yl)indazole (20) as yellow solid (150 mg, Yield: 100%).

### Synthesis of 3-isopropyl-2-methyl-5-[2-(methylthio)pyrimidin-4-yl]-1H-indazole (30)

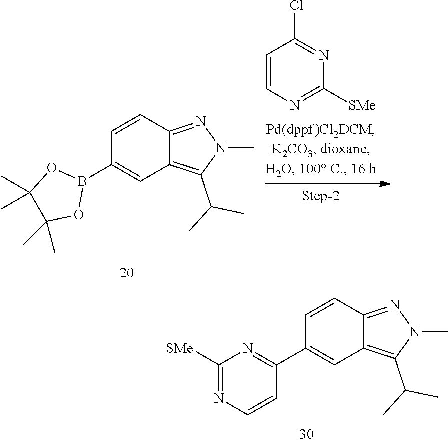

To a stirred solution of 3-isopropyl-2-methyl-5-(4,4,5,5-tetramethyl-1,3,2-dioxaborolan-2-yl)indazole (20) (150 mg, 0.50 mmol, 1 eq) in 1,4-dioxane and water (3:1) (8 mL) in a microwave vial was added 4-chloro-2-(methylthio)pyrimidine (96 mg, 0.60 mmol, 1.2 eq), K₂CO₃ (173 mg, 0.25 mmol, 2.5 eq), Pd(dppf)Cl₂DCM (20 mg, 0.025 mmol, 0.05 eq) and degassed for 5 mins with argon. Then the reaction mixture was stirred at 100° C. for 16 h. The reaction mixture was diluted with water and ethyl acetate and the resulting slurry was filtered through a pad of Celite® (i.e., diatomaceous earth). The organic layer was separated, washed with brine solution, dried over sodium sulfate and concentrated to provide crude product which was purified by Combiflash Chromatography (4 g column) to afford 3-isopropyl-2-methyl-5-[2-(methylthio)pyrimidin-4-yl]-1H-indazole (30) as a yellow solid (130 mg, yield: 87%). TLC system: Hexane: EtOAc (2:1), R_{f} value: ˜0.2.

### Synthesis of mixture of 3-isopropyl-2-methyl-5-(2-methylsulfinylpyrimidin-4-yl)indazole and 3-isopropyl-2-methyl-5-(2-methylsulfonylpyrimidin-4-yl)indazole (40)

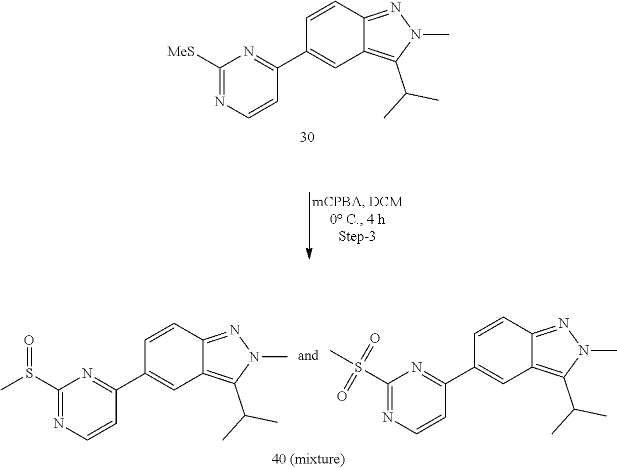

To a stirred solution of 3-isopropyl-2-methyl-5-[2-(methylthio)pyrimidin-4-yl]-1H-indazole (30) (98 mg, 0.33 mmol, 1.0 eq) in DCM (4 mL) cooled to 0° C. and added 3-chloroperbenzoic acid (mCPBA) (purity, 77%) (96 mg, 0.43 mmol, 1.3 eq). The reaction mixture was stirred at 0° C. for 4 h. After completion of reaction by TLC, reaction mixture was quenched with sat aq. NaHCO₃ solution (10 mL) and extracted with ethyl acetate. The combined organic layers were washed with brine solution, dried over sodium sulfate and concentrated to afford crude mixture of 3-isopropyl-2-methyl-5-(2-methylsulfinylpyrimidin-4-yl)indazole and 3-isopropyl-2-methyl-5-(2-methylsulfonylpyrimidin-4-yl)indazole (40) as a yellow solid (104 mg, yield: 100%). TLC system: EtOAc (100%) R_{f} value: ˜0.01 and 0.2.

### Synthesis of tert-butyl (1S, 3R)-[3-[4-(3-isopropyl-2-methylindazol-5-yl)pyrimidin-2-yl]aminocyclopentan-1-yl]aminocarboxylate (50a)

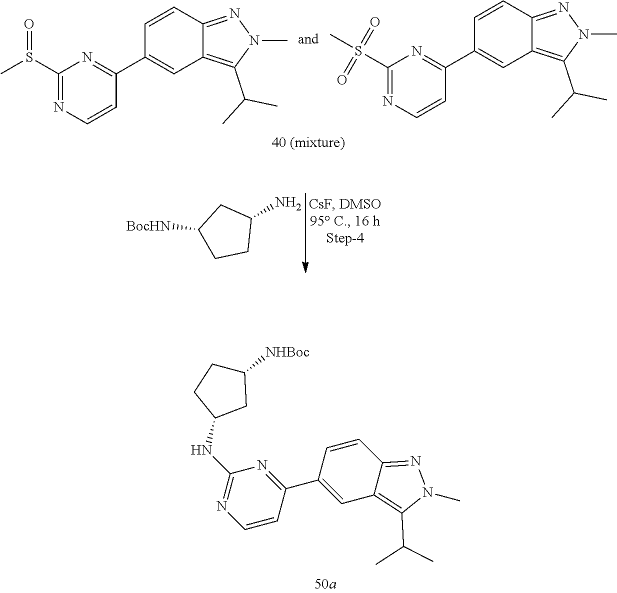

To a stirred solution of mixture of 3-isopropyl-2-methyl-5-(2-methylsulfinylpyrimidin-4-yl)indazole and 3-isopropyl-2-methyl-5-(2-methylsulfonylpyrimidin-4-yl)indazole (40) (30 mg, 0.10 mmol, 1 eq) in DMSO (5 mL) at room temperature was added (1S,3S)-3-amino-1-(BOC-amino)cyclopentane (28 mg, 0.14 mmol, 1.5 eq) and cesium fluoride (21 mg, 0.14 mmol, 1.5 eq). Then the reaction mixture was stirred at 95° C. for 16 h. After completion of reaction by TLC, reaction mixture was cooled to room temperature and extracted with ethyl acetate. The organic layer was washed with brine solution, dried over anhydrous sodium sulfate and then concentrated to provide product which was purified by Combiflash Chromatography (4 g column) to afford tert-butyl (1S, 3R)-[3-[4-(3-isopropyl-2-methylindazol-5-yl)pyrimidin-2-yl]aminocyclopentan-1-yl]aminocarboxylate (50a) as yellow solid (23 mg, 53%). TLC system: Hexane: EtOAc (1:1), R_{f} value: ˜0.2.

### Synthesis of (1S, 3R)-[3-[4-(3-isopropyl-2-methylindazol-5-yl)pyrimidin-2-yl]aminocyclopentan-1-yl]-1,3-diamine Trifluoroacetate (Compound I-1)

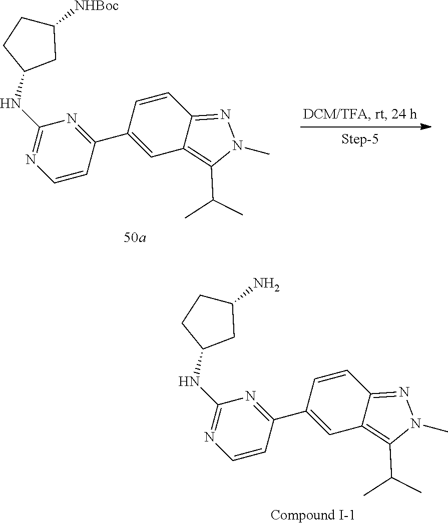 [SMILES: CC(C)c1c2cc(-c3ccnc(N[C@@H]4CC[C@H](NC(=O)OC(C)(C)C)C4)n3)ccc2nn1C ; CC(C)c1c2cc(-c3ccnc(N[C@@H]4CC[C@H](N)C4)n3)ccc2nn1C]

To a stirred solution of tert-butyl (1S, 3R)-[3-[4-(3-isopropyl-2-methylindazol-5-yl)pyrimidin-2-yl]aminocyclopentan-1-yl]aminocarboxylate (50a) (22 mg, 0.05 mmol, 1 eq) in DCM (4 mL) at room temperature was added trifluoroacetic acid (TFA) (2 mL) and the reaction mixture was stirred at room temperature for 24 h. After completion of reaction by TLC, the solvent was removed in vacuum. Then diethyl ether was added to the reaction and a lot of yellow solid will appear in the reaction. The mixture was centrifuged, washed over diethyl ether and dried at room temperature to provide the product, (1S, 3R)-[3-[4-(3-isopropyl-2-methylindazol-5-yl)pyrimidin-2-yl]aminocyclopentan-1-yl]-1,3-diamine Trifluoroacetate (Compound I-1) as yellow solid (25 mg, 100%). ¹HNMR (400 MHz, MeOD-d4) δ 8.82 (s, 1H), 8.29 (d, J=6.0 Hz, 1H), 8.14 (d, J=9.0 Hz, 1H), 7.64 (d, J=9.0 Hz, 1H), 754 (d, J=6.0 Hz, 1H), 4.59 (broad, 1H), 4.19 (s, 3H), 3.76-3.74 (m, 2H), 2.79-2.73 (m, 1H), 2.30-2.20 (m, 2H), 2.00-1.87 (m, 2H), 1.80-1.72 (m, 1H), 1.60 (d, J=6.8 Hz, 6H); HRMS (ESI) m/z: [M+H]⁺ calcd for C₂₀H₂₆N₆, 351.2292, found 351.2270.

### Example 2

### Synthesis of Compound I-2

A precursor 3-isopropyl-2-methyl-5-(2-methylsulfinylpyrimidin-4-yl)indazole and 3-isopropyl-2-methyl-5-(2-methylsulfonylpyrimidin-4-yl)indazole (40) was prepared according to the syntheses described in EXAMPLE 1 following the steps 1-3.

### Synthesis of tert-butyl (1S, 3S)-[3-[4-(3-isopropyl-2-methylindazol-5-yl)pyrimidin-2-yl]aminocyclopentan-1-yl]aminocarboxylate (50b)

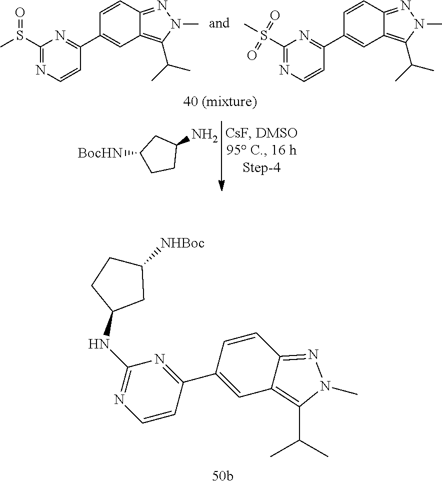

To a stirred solution of mixture of 3-isopropyl-2-methyl-5-(2-methylsulfinylpyrimidin-4-yl)indazole and 3-isopropyl-2-methyl-5-(2-methylsulfonylpyrimidin-4-yl)indazole (40) (30 mg, 0.10 mmol, 1 eq) in DMSO (5 mL) at room temperature was added (1S,3S)-3-amino-1-(BOC-amino)cyclopentane (28 mg, 0.14 mmol, 1.5 eq) and cesium fluoride (21 mg, 0.14 mmol, 1.5 eq). Then the reaction mixture was stirred at 95° C. for 16 h. After completion of reaction by TLC, the reaction mixture was cooled to room temperature and extracted with ethyl acetate. The organic layer was washed with brine solution, dried over anhydrous sodium sulfate and then concentrated to provide product which was purified by Combiflash Chromatography (4 g column) to afford tert-butyl (1S, 3S)-[3-[4-(3-isopropyl-2-methylindazol-5-yl)pyrimidin-2-yl]aminocyclopentan-1-yl]aminocarboxylate (50b) as yellow solid (25 mg, 58%). TLC system: Hexane: EtOAc (1:1), R_{f} value: ˜0.2.

### Synthesis of (1S, 3S)-[3-[4-(3-isopropyl-2-methylindazol-5-yl)pyrimidin-2-yl]aminocyclopentan-1-yl]-1,3-diamine Trifluoroacetate (Compound I-2)

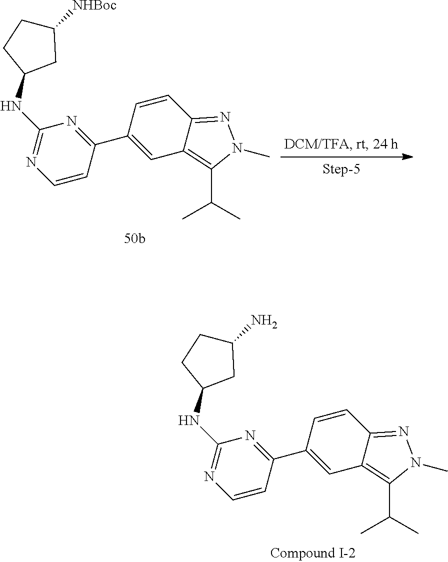 [SMILES: CC(C)c1c2cc(-c3ccnc(N[C@H]4CC[C@H](NC(=O)OC(C)(C)C)C4)n3)ccc2nn1C ; CC(C)c1c2cc(-c3ccnc(N[C@H]4CC[C@H](N)C4)n3)ccc2nn1C]

To a stirred solution of tert-butyl (1S, 3S)-[3-[4-(3-isopropyl-2-methylindazol-5-yl)pyrimidin-2-yl]aminocyclopentan-1-yl]aminocarboxylate (50b) (25 mg, 0.056 mmol, 1 eq) in DCM (4 mL) at room temperature was added trifluoroacetic acid (TFA) (2 mL) and the reaction mixture was stirred at room temperature for 24 h. After completion of reaction by TLC, the solvent was removed in vacuum. Then diethyl ether was added to the reaction and a lot of yellow solid will appear in the reaction. The mixture was centrifuged, washed over diethyl ether and dried at room temperature to provide the product, (1S, 3S)-[3-[4-(3-isopropyl-2-methylindazol-5-yl)pyrimidin-2-yl]aminocyclopentan-1-yl]-1,3-diamine Trifluoroacetate (Compound I-2) as yellow solid (22 mg, 69%). ¹HNMR (400 MHz, MeOD-d4) δ 8.79 (s, 1H), 8.28 (d, J=6.2 Hz, 1H), 8.15 (d, J=9.1 Hz, 1H), 7.64 (d, J=9.1 Hz, 1H), 7.52 (s, broad, 1H), 4.71 (s, broad, 1H), 4.19 (s, 3H), 3.89-3.82 (m, 1H), 3.70-3.62 (m, 1H), 2.44-2.34 (m, 2H), 2.30-2.18 (m, 2H), 1.92-1.84 (m, 1H), 1.79-1.70 (m, 1H), 1.60 (d, J=7.0 Hz, 6H); HRMS (ESI) m/z: [M+H]⁺ calcd for C₂₀H₂₆N₆, 351.2292, found 351.2265.

### Example 3

### Synthesis of Compound I-3

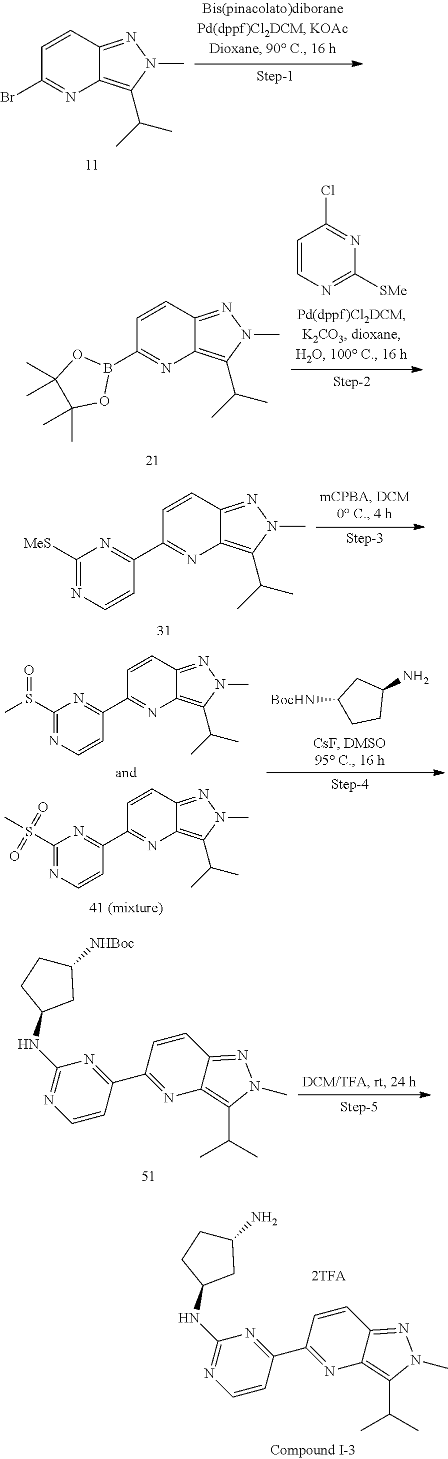

### Synthesis of 3-isopropyl-2-methyl-5-(4,4,5,5-tetramethyl-1,3,2-dioxaborolan-2-yl) pyrazolo[4,3-b]pyridine (21)

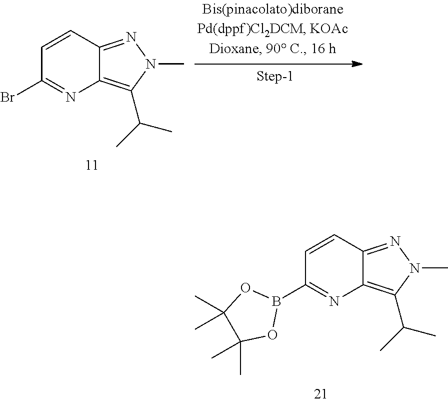

To a stirred solution of 5-bromo-2methyl-3-propylpyrazolo[4,3-b]pyridine (11) (45 mg, 0.18 mmol, 1.0 eq) and bis(pinacolato)diborane (56 mg, 0.22 mmol, 1.2 eq) in 1,4-dioxane (8 mL) in a microwave vial was added potassium acetate (44 mg, 0.45 mmol, 2.5 eq), Pd(dppf)Cl₂·DCM (8 mg, 0.01 mmol, 0.05 eq). Then, the vial was sealed with the cap and degassed for 5 min with argon. The reaction mixture was stirred at 90° C. for 16 h. After completion of reaction by TLC, reaction mixture was diluted with water and ethyl acetate, resulting slurry was filtered through a pad of Celite® (i.e., diatomaceous earth). The organic layer was separated, washed with brine solution, dried over sodium sulfate and concentrated to provide crude product, 3-isopropyl-2-methyl-5-(4,4,5,5-tetramethyl-1,3,2-dioxaborolan-2-yl)pyrazolo[4,3-b]pyridine (21) as brown oil (54 mg, Yield: 100%).

### Synthesis of 3-isopropyl-2-methyl-5-[2-(methylthio)pyrimidin-4-yl]pyrazolo[4,3-b]pyridine (31)

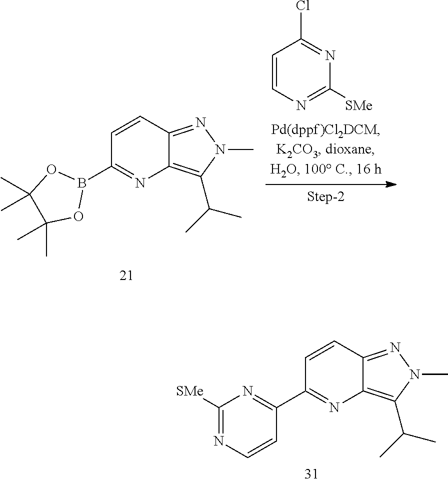

To a stirred solution of 3-isopropyl-2-methyl-5-(4,4,5,5-tetramethyl-1,3,2-dioxaborolan-2-yl)pyrazolo[4,3-b]pyridine (21) (54 mg, 0.18 mmol, 1 eq) in 1,4-dioxane and water (3:1) (8 mL) in a microwave vial was added 4-chloro-2-(methylthio)pyrimidine (43 mg, 0.27 mmol, 1.5 eq), K₂CO₃ (62 mg, 0.45 mmol, 2.5 eq), Pd(dppf)Cl₂DCM (8 mg, 0.01 mmol, 0.05 eq) and degassed for 5 min with argon. Then the reaction mixture was stirred at 100° C. for 16 h. The reaction mixture was diluted with water and ethyl acetate and the resulting slurry was filtered through a pad of Celite® (i.e., diatomaceous earth). The organic layer was separated, washed with brine solution, dried over sodium sulfate and concentrated to provide crude product which was purified by Combiflash Chromatography (4 g column) to afford 3-isopropyl-2-methyl-5-[2-(methylthio)pyrimidin-4-yl]pyrazolo[4,3-b]pyridine (31) as a yellow solid (10 mg, yield: 18%). TLC system: Hexane: EtOAc (2:1), R_{f} value: ˜0.3.

### Synthesis of mixture of 3-isopropyl-2-methyl-5-(2-methylsulfinylpyrimidin-4-yl)pyrazolo[4,3-b]pyridine and 3-isopropyl-2-methyl-5-(2-methylsulfonylpyrimidin-4-yl)pyrazolo[4,3-b]pyridine (41)

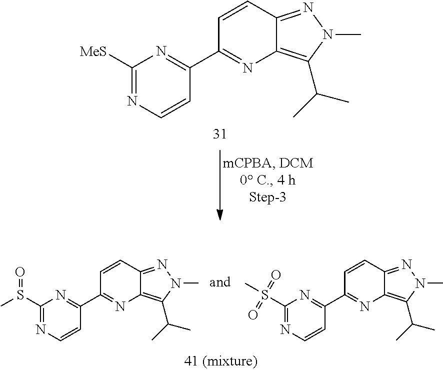

To a stirred solution of 3-isopropyl-2-methyl-5-[2-(methylthio)pyrimidin-4-yl]pyrazolo[4,3-b]pyridine (31) (10 mg, 0.033 mmol, 1.0 eq) in DCM (4 mL) cooled to 0° C. and added 3-chloroperbenzoic acid (mCPBA) (purity, 77%) (10 mg, 0.043 mmol, 1.3 eq). The reaction mixture was stirred at 0° C. for 4 h. After completion of reaction by TLC, reaction mixture was quenched with sat. aq. NaHCO₃ solution (10 mL) and extracted with ethyl acetate. The combined organic layers were washed with brine solution, dried over sodium sulfate and concentrated to afford crude mixture of 3-isopropyl-2-methyl-5-(2-methylsulfinylpyrimidin-4-yl)pyrazolo[4,3-b]pyridine and 3-isopropyl-2-methyl-5-(2-methylsulfonylpyrimidin-4-yl)pyrazolo[4,3-b]pyridine (41) as a yellow solid (10 mg, yield: 100%). TLC system: EtOAc (100%) R_{f} value: ˜0.01 and 0.2.

### Synthesis of tert-butyl (1S, 3S)-[3-[4-[3-isopropyl-2-methylpyrazolo[4,3-b]pyridin-5-yl]pyrimidin-2-yl]aminocyclopentan-1-yl]aminocarboxylate (51)

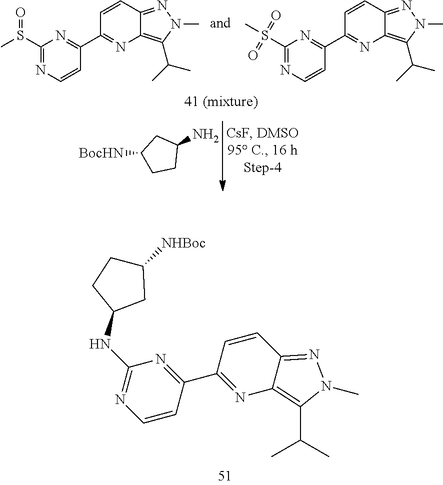

To a stirred solution of mixture of 3-isopropyl-2-methyl-5-(2-methylsulfinylpyrimidin-4-yl)pyrazolo[4,3-b]pyridine and 3-isopropyl-2-methyl-5-(2-methylsulfonylpyrimidin-4-yl)pyrazolo[4,3-b]pyridine (41) (10 mg, 0.033 mmol, 1 eq) in DMSO (5 mL) at room temperature was added (1S,3S)-3-amino-1-(BOC-amino)cyclopentane (10 mg, 0.05 mmol, 1.5 eq) and cesium fluoride (8 mg, 0.05 mmol, 1.5 eq). Then the reaction mixture was stirred at 95° C. for 16 h. After completion of reaction by TLC, the reaction mixture was cooled to room temperature and extracted with ethyl acetate. The organic layer was washed with brine solution, dried over anhydrous sodium sulfate and then concentrated to provide product which was purified by Combiflash Chromatography (4 g column) to afford tert-butyl (1S, 3S)-[3-[4-[3-isopropyl-2-methylpyrazolo[4,3-b]pyridin-5-yl]pyrimidin-2-yl]aminocyclopentan-1-yl]aminocarboxylate (51) as yellow solid (10 mg, 67%). TLC system: Hexane: EtOAc (1:1), R_{f} value: ˜0.2.

### Synthesis of (1S, 3S)-[3-[4-[3-isopropyl-2-methylpyrazolo[4,3-b]pyridin-5-yl]pyrimidin-2-yl]aminocyclopentan-1-yl]-1,3-diamine Trifluoroacetate (Compound I-17)

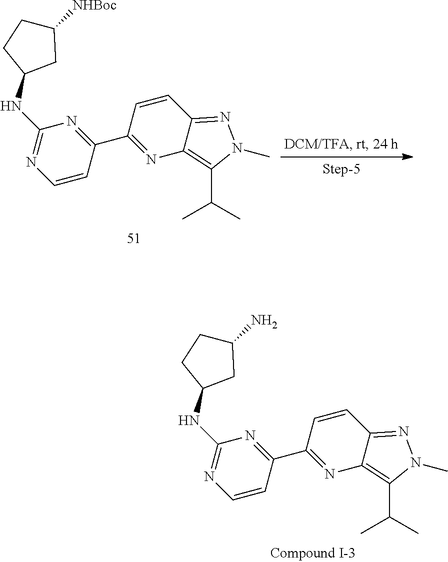 [SMILES: CC(C)c1c2nc(-c3ccnc(N[C@H]4CC[C@H](NC(=O)OC(C)(C)C)C4)n3)ccc2nn1C ; CC(C)c1c2nc(-c3ccnc(N[C@H]4CC[C@H](N)C4)n3)ccc2nn1C]

To a stirred solution of tert-butyl (1S, 3S)-[3-[4-[3-isopropyl-2-methylpyrazolo[4,3-b]pyridin-5-yl]pyrimidin-2-yl]aminocyclopentan-1-yl]aminocarboxylate (51) (10 mg, 0.022 mmol, 1 eq) in DCM (4 mL) at room temperature was added trifluoroacetic acid (TFA) (2 mL) and the reaction mixture was stirred at room temperature for 24 h. After completion of reaction by TLC, the solvent was removed in vacuum. Then diethyl ether was added to the reaction and a lot of yellow solid will appear in the reaction. The mixture was centrifuged, washed over diethyl ether and dried at room temperature to provide the product, (1S, 3S)-[3-[4-[3-isopropyl-2-methylpyrazolo[4,3-b]pyridin-5-yl]pyrimidin-2-yl]aminocyclopentan-1-yl]-1,3-diamine Trifluoroacetate (Compound I-3) as yellow solid (5 mg, 38%). ¹HNMR (400 MHz, MeOD-d4) δ 8.53 (d, J=8.9 Hz, 1H), 8.40 (d, J=5.6 Hz, 1H), 8.05 (d, J=8.9 Hz, 1H), 7.86 (J=5.6 Hz, 1H), 4.68 (s, broad, 1H), 4.07 (s, 3H), 3.87-3.81 (m, 1H), 3.64-3.57 (m, 1H), 2.41-2.33 (m, 2H), 2.23-2.19 (m, 2H), 1.85-1.72 (m, 2H), 1.54 (d, J=7.0 Hz, 6H); HRMS data: HRMS (ESI) m/z [M+H]+ calcd for C₁₉H₂₅N₇, 352.2244, found: 352.2217.

### Example 4

### Synthesis of Compound I-4

Compound I-4 was prepared from a fluorine substituted derivative of 3-isopropyl-2-methyl-5-(4,4,5,5-tetramethyl-1,3,2-dioxaborolan-2-yl)indazole (20) followed by the same syntheses described in EXAMPLE 2.

### Example 5

### Synthesis of Compound I-5

Compound I-21 was prepared from a fluorine substituted derivative of 5-bromo-2methyl-3-propylpyrazolo[4,3-b]pyridine (11) followed by the same syntheses described in EXAMPLE 3.

### Example 6

### Synthesis of Compound I-6

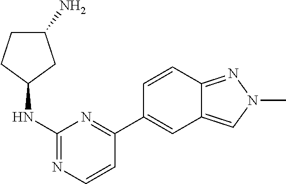 [SMILES: Cn1cc2cc(-c3ccnc(N[C@H]4CC[C@H](N)C4)n3)ccc2n1]

Overall Reaction Scheme:

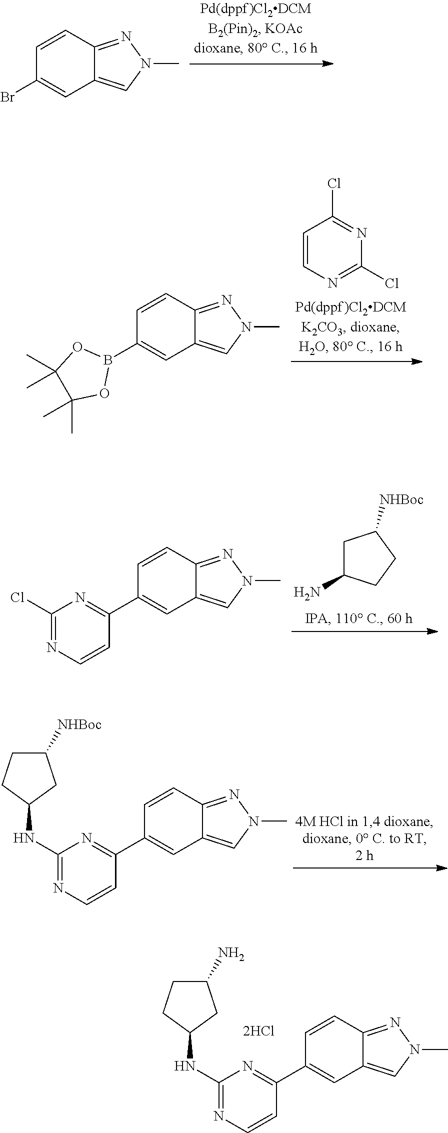

### Synthesis of 2-methyl-5-(4,4,5,5-tetramethyl-1,3,2-dioxaborolan-2-yl)-2H-indazole

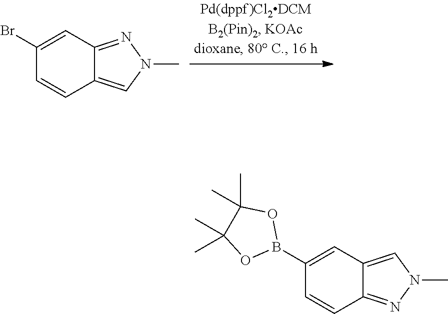

A solution of 5-bromo-2-methyl-2H-indazole (500 mg, 2.36 mmol, 1.0 eq), bis(pinacolato)diborane (722 mg, 2.84 mmol, 1.2 eq) and potassium acetate (582 mg, 5.92 mmol, 2.5 eq), in 1,4-dioxane (10 mL) was degassed for 5 min and added Pd(dppf)Cl₂·DCM (97 mg, 0.118 mmol, 0.05 eq). Degassed for additional 5 min with nitrogen gas and stirred at 80° C. for 16 h. After completion of reaction by TLC, diluted with water (50 mL) and extracted with ethyl acetate (2×50 mL). Combined organic layer was washed with brine solution (20 mL), dried over sodium sulfate and concentrated to afford 2-methyl-5-(4,4,5,5-tetramethyl-1,3,2-dioxaborolan-2-yl)-2H-indazole as a black semi solid (630 mg, crude). TLC system: EtOAc/petroleum ether (40:60), R_{f} value: ˜0.45 (single spot and expected as boronate ester). Cr: LC/MS: 74.5% of 177.1 [M+H] as boronic acid and 18% of 259.2 [M+H] as boronate ester.

### Synthesis of 5-(2-chloropyrimidin-4-yl)-2-methyl-2H-indazole

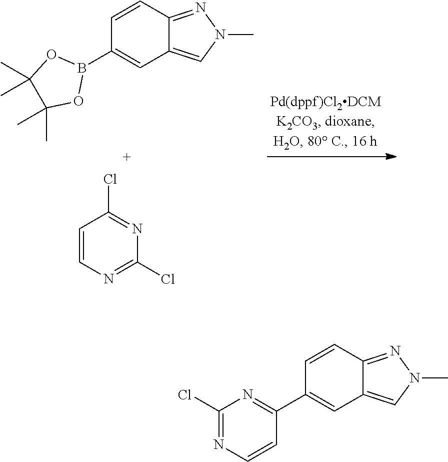

In a sealed tube, to a stirred solution of 2-methyl-5-(4,4,5,5-tetramethyl-1,3,2-dioxaborolan-2-yl)-2H-indazole (550 mg, 2.13 mmol, 1 eq) in 1,4-dioxane and water (3:1) (11 mL) at room temperature was added 2,4-dichloropyrimidine (381 mg, 2.58 mmol, 1.2 eq) and K₂CO₃ (735 mg, 5.32 mmol, 2.5 eq). Degassed for 5 mins and later added Pd(dppf)Cl₂·DCM (87 mg, 0.106 mmol, 0.05 eq). Reaction mixture was stirred at 80° C. for 16 h. After completion of reaction by TLC, diluted with water and extracted with ethyl acetate (2×50 mL). Combined organic layer was dried over sodium sulfate, concentrated and purified by silica gel column (60-120 mesh, eluent: 50% Ethyl Acetate/Petroleum ether) to afford 5-(2-chloropyrimidin-4-yl)-2-methyl-2H-indazole (450 mg, 88%) as light-yellow solid. TLC system: EtOAc (100%), R_{f} value: ˜0.2. LC/MS Retention time=2.92 min, 245.0 [M+H]⁺.

¹H NMR (400 MHz, CDCl₃) δ 8.6 (d, J=5.2 Hz, 1H), 8.60-8.59 (m, 1H), 8.06 (s, 1H), 7.94 (dd, J=9.2 & 1.6 Hz, 1H), 7.79 (dd, J=8.8 & 0.8 Hz, 1H), 7.67 (d, J=5.2 Hz, 1H), 4.27 (s, 3H).

### Synthesis of tert-butyl ((1S,3S)-3-((4-(2-methyl-2H-indazol-5-yl)pyrimidin-2-yl)amino)cyclopentyl) carbamate

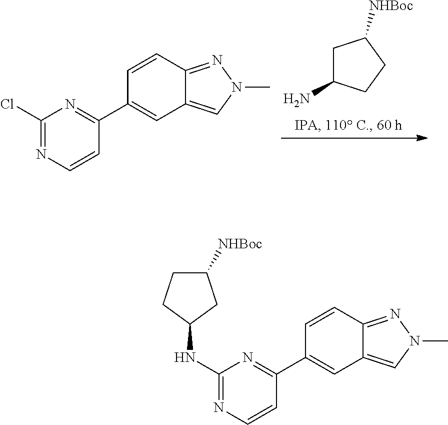

To a stirred solution of 5-(2-chloropyrimidin-4-yl)-2-methyl-2H-indazole (450 mg, 1.84 mmol, 1 eq) in IPA (5 mL) at room temperature was added tert-butyl ((1S,3S)-3-aminocyclopentyl)carbamate (443 mg, 2.21 mmol, 1.2 eq) and stirred at 110° C. for 60 h. TLC showed polar spot and the reaction mixture was allowed to room temperature. The reaction mixture was evaporated and diluted with water (30 mL) and extracted with EtOAc (2×30 ml). Organic layer was dried over Na₂SO₄, filtered, evaporated and purified by reverse phase column (eluted in 25% to 35% acetonitrile and 0.01% FA in Water) to afford tert-butyl ((1S,3S)-3-((4-(2-methyl-2H-indazol-5-yl)pyrimidin-2-yl)amino)cyclopentyl)carbamate (235 mg, 32%) as an off white solid. TLC system: EtOAc/petroleum ether (70:30), R_{f} value: ˜0.40; LC/MS Retention time=3.0 min, 409.2 [M+H]⁺.

¹H NMR (400 MHz, CDCl₃): δ 8.39 (s, 1H), 8.31 (d, J=5.2 Hz, 1H), 8.01 (s, 1H), 7.95 (d, J=8.0 Hz, 1H), 7.75 (d, J=9.2 Hz, 1H), 7.01 (d, J=5.6 Hz, 1H), 5.21 (d, J=6.8 Hz, 1H), 4.59 (brs, 1H), 4.53-4.48 (m, 1H), 4.25 (s, 3H), 4.18-4.11 (m, 1H), 2.34-2.20 (m, 2H), 2.04-1.98 (m, 2H), 1.77-1.48 (m, 2H), 1.45 (s, 9H).

### Synthesis of (1S,3S)—N-(4-(2-methyl-2H-indazol-5-yl)pyrimidin-2-yl)cyclopentane-1,3-diamine HCl salt

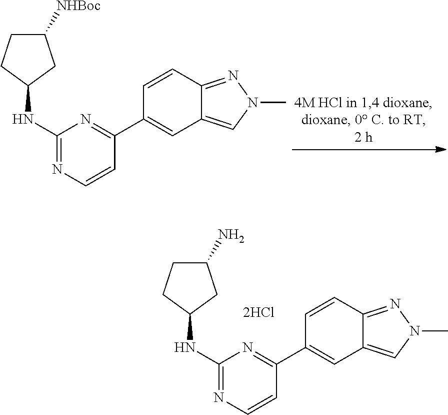 [SMILES: Cn1cc2cc(-c3ccnc(N[C@H]4CC[C@H](NC(=O)OC(C)(C)C)C4)n3)ccc2n1 ; Cn1cc2cc(-c3ccnc(N[C@H]4CC[C@H](N)C4)n3)ccc2n1]

To a stirred solution of tert-butyl ((1S,3S)-3-((4-(2-methyl-2H-indazol-5-yl)pyrimidin-2-yl)amino)cyclopentyl)carbamate (230 mg, 0.56 mmol, 1 eq.) in 1,4-Dioxane (2 mL) at 0° C., added 4M HCl in 1,4-Dioxane (0.4 mL) and stirred for 2 h at room temperature. After completion of starting material, reaction mixture was evaporated and triturated with diethyl ether (2×3 mL) followed by lyophilization afforded (1S,3S)—N1-(4-(2-methyl-2H-indazol-5-yl)pyrimidin-2-yl)cyclopentane-1,3-diamine HCl salt (120 mg, 56%) as a yellow solid. TLC system: MeOH/DCM (20:80), R_{f} value: ˜0.1; LC/MS Retention time=1.88 min, 309.3 [M+H]⁺.

¹H NMR (400 MHz, CD₃OD) δ 8.90 (brs, 1H), 8.62 (s, 1H), 8.31-8.28 (m, 2H), 7.77 (d, J=9.2 Hz, 1H), 7.67 (d, J=6.8 Hz, 1H), 4.93-4.89 (m, 1H), 4.31 (s, 3H), 3.92-3.87 (m, 1H), 2.47-2.36 (m, 2H), 2.32-2.28 (m, 2H), 1.94-1.86 (m, 2H).

### Example 7

### Synthesis of Compound I-7

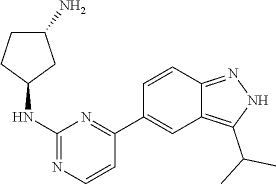 [SMILES: CC(C)c1[nH]nc2ccc(-c3ccnc(N[C@H]4CC[C@H](N)C4)n3)cc12]

Overall Reaction Scheme:

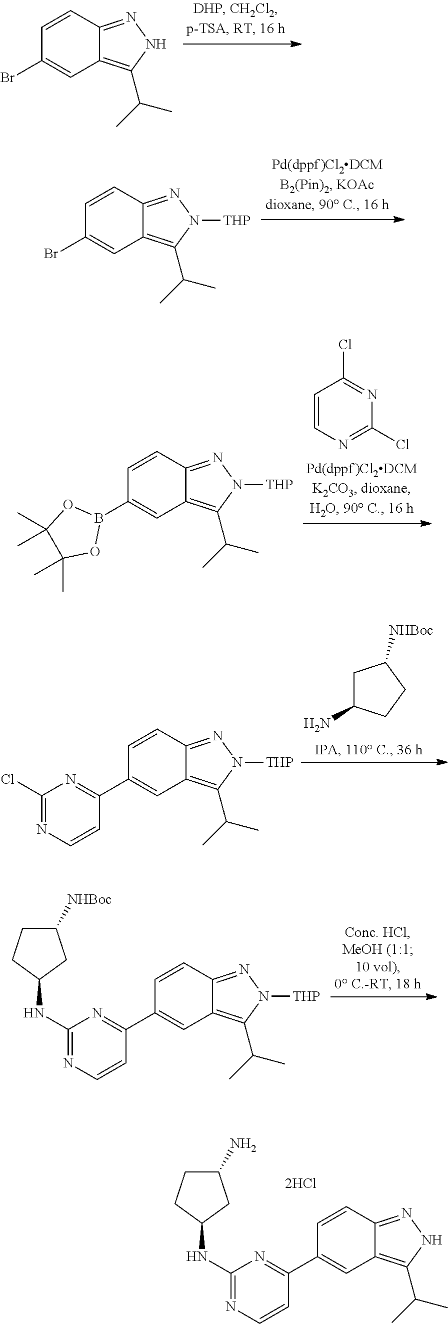

### Synthesis of 5-bromo-3-isopropyl-2-(tetrahydro-2H-pyran-2-yl)-2H-indazole

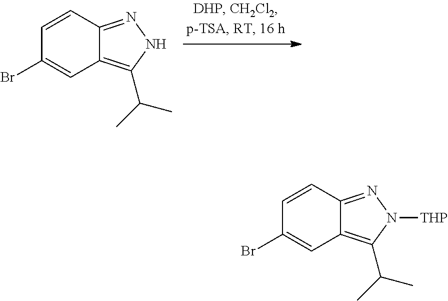

To a stirred solution of 5-bromo-3-isopropyl-2H-indazole (200 mg, 0.84 mmol, 1.0 eq), in DCM (4 mL) at room temperature was added dihydropyran (0.2 mL, 1.67 mmol, 2.0 eq), p-TSA (8 mg, 0.04 mmol, 0.05 eq) and stirred for 16 h. After completion of reaction by TLC, diluted with cold water (30 mL) and DCM (2×30 mL). Organic layer was dried over sodium sulfate, filtered, concentrated and purified by silica gel column (60-120 mesh, eluent: 15% EtOAc/petroleum ether) to afford 5-bromo-3-isopropyl-2-(tetrahydro-2H-pyran-2-yl)-2H-indazole as a yellow liquid (260 mg, Yield: 96%). TLC system: EtOAc/petroleum ether (20:80), R_{f} value: ˜0.8. LC/MS Retention time=4.48 min, 323.0 [M+H]⁺.

¹H NMR (400 MHz, CDCl₃) δ 7.86 (t, J=1.2 Hz, 1H), 7.42-7.41 (m, 2H), 5.61 (dd, J=9.6 & 2.4 Hz, 1H), 4.06-4.03 (m, 1H), 3.75-3.69 (m, 1H), 3.37-3.30 (m, 1H), 2.53-2.49 (m, 1H), 2.14-2.12 (m, 1H), 2.04-2.00 (m, 1H), 1.77-1.71 (m, 2H), 1.65-1.62 (m, 1H), 1.44 (d, J=7.6 Hz, 6H).

### Synthesis of 3-isopropyl-2-(tetrahydro-2H-pyran-2-yl)-5-(4,4,5,5-tetramethyl-1,3,2-dioxaborolan-2-yl)-2H-indazole

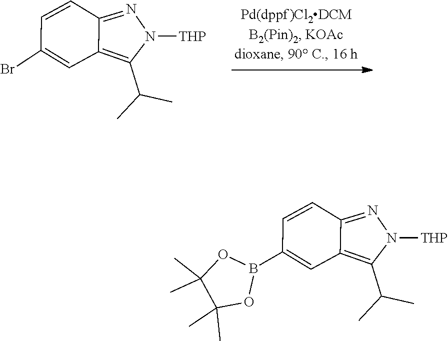

To a stirred solution of 5-bromo-3-isopropyl-2-(tetrahydro-2H-pyran-2-yl)-2H-indazole (260 mg, 0.87 mmol, 1.0 eq), bis(pinacolato)diborane (224 mg, 1.04 mmol, 1.2 eq) and potassium acetate (213 mg, 2.17 mmol, 2.5 eq), in 1,4-dioxane (6 mL) was degassed for 5 min and added Pd(dppf)Cl₂·DCM (36 mg, 0.043 mmol, 0.05 eq). The reaction mixture was stirred at 90° C. for 16 h. After completion of reaction by TLC, diluted with water (50 mL). and extracted with ethyl acetate (2×50 mL). Organic layer was dried over sodium sulfate and concentrated to afford 3-isopropyl-2-(tetrahydro-2H-pyran-2-yl)-5-(4,4,5,5-tetramethyl-1,3,2-dioxaborolan-2-yl)-2H-indazole (325 mg, crude) as black semi solid. TLC system: EtOAc/petroleum ether (30:70), R_{f} value: ˜0.5. LC/MS Retention time=4.68 min, 371.2 [M+H]⁺. Cr: LC/MS: 62.1% boronate ester m/z and 10% in boronic acid m/z observed, material was taken forward to next step without purification.

### Synthesis of 5-(2-chloropyrimidin-4-yl)-3-isopropyl-2-(tetrahydro-2H-pyran-2-yl)-2H-indazole

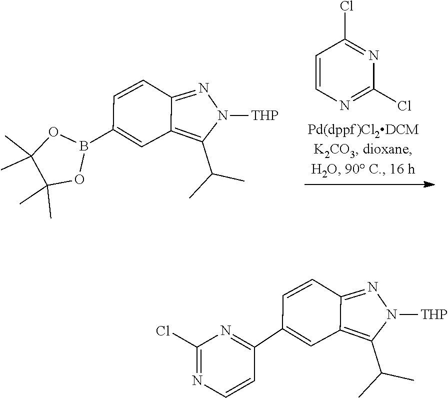

To a degassed stirred solution of 3-isopropyl-2-(tetrahydro-2H-pyran-2-yl)-5-(4,4,5,5-tetramethyl-1,3,2-dioxaborolan-2-yl)-2H-indazole (320 mg, 0.86 mmol, 1.0 eq) in 1,4-dioxane and water (3:1) (6 mL) was added 2,4-dichloropyrimidine (154 mg, 1.04 mmol, 1.2 eq), K₂CO₃ (298 mg, 2.16 mmol, 2.5 eq) followed by Pd(dppf)Cl₂DCM (35 mg, 0.043 mmol, 0.05 eq) and stirred at 90° C. for 16 h. After completion of reaction by TLC, diluted with water and extracted with ethyl acetate (2×50 mL). Combined organic layer was dried over sodium sulfate, concentrated and purified by silica gel column (60-120 mesh, eluent: 25% EtOAc/petroleum ether) to afford 5-(2-chloropyrimidin-4-yl)-3-isopropyl-2-(tetrahydro-2H-pyran-2-yl)-2H-indazole (280 mg, 70% w.r.t to purity) as white solid. TLC system: EtOAc/petroleum ether (30:70), R_{f} value: ˜0.2; LC/MS Retention time=4.77 min, 357.2 [M+H]⁺; 76% purity

### Synthesis of tert-butyl ((1S,3S)-3-((4-(3-isopropyl-2-(tetrahydro-2H-pyran-2-yl)-2H-indazol-5-yl)pyrimidin-2-yl)amino)cyclopentyl)carbamate

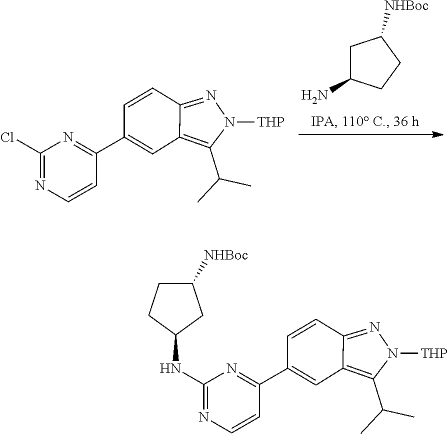

To a stirred solution of 5-(2-chloropyrimidin-4-yl)-3-isopropyl-2-(tetrahydro-2H-pyran-2-yl)-2H-indazole (280 mg, 0.79 mmol, 1 eq) in IPA (4.5 mL) was added tert-butyl ((1S,3S)-3-aminocyclopentyl)carbamate (190 mg, 0.94 mmol, 1.2 eq) and stirred at 110° C. for 36 h. TLC showed polar spot and the reaction mixture was allowed to room temperature. The reaction mixture was evaporated and diluted with water (30 mL) and extracted with EtOAc (3×20 ml). Organic layer was dried over Na₂SO₄, filtered, evaporated and purified by silica gel column (60-120 mesh, eluent: 15% to 70% EtOAc/petroleum ether) to afford tert-butyl ((1S,3S)-3-((4-(3-isopropyl-2-(tetrahydro-2H-pyran-2-yl)-2H-indazol-5-yl)pyrimidin-2-yl)amino) cyclopentyl)carbamate (150 mg, yield: 37%) as yellow gummy liquid. TLC system: EtOAc/Petroleum ether (70:30), R_{f} value: ˜0.20; LC/MS Retention time=4.67 min, 521.3 [M+H]⁺.

### Synthesis of (1S,3S)—N1-(4-(3-isopropyl-2H-indazol-5-yl)pyrimidin-2-yl)cyclopentane-1,3-diamine HCl salt

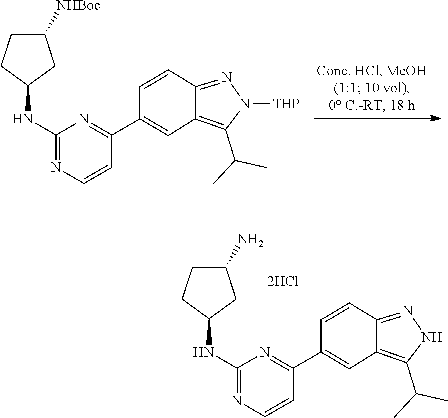

To a stirred solution of ((1S,3S)-3-((4-(3-isopropyl-2-(tetrahydro-2H-pyran-2-yl)-2H-indazol-5-yl)pyrimidin-2-yl)amino) cyclopentyl)carbamate (150 mg, 0.29 mmol, 1 eq.) in MeOH (0.7 mL) at 0° C., added concentrated HCl (0.7 mL) and stirred at room temperature for 18 h. After completion of starting material, volatiles removed, triturated with diethyl ether (2×5 mL) and purified by reverse phase column (Eluent: 8% ACN and 0.01% FA in water) to afford (1S,3S)—N1-(4-(3-isopropyl-2H-indazol-5-yl)pyrimidin-2-yl)cyclopentane-1,3-diamine HCl salt (28 mg, yield: 29%) as yellow gummy solid. TLC system: MeOH/DCM (30:70), R_{f} value: ˜0.1; LC/MS Retention time=2.32 min, 337.4 [M+H]⁺.

¹H NMR (400 MHz, CD₃OD) δ 8.84 (s, 1H), 8.38 (brs, 1H), 8.29 (d, J=6.8 Hz, 1H), 7.73 (d, J=6.4 Hz, 1H), 7.65 (d, J=9.2 Hz, 1H), 3.91-3.87 (m, 1H), 3.59-3.53 (m, 1H), 3.35-3.33 (m, 1H), 2.48-2.36 (m, 2H), 2.32-2.27 (m, 2H), 1.93-1.80 (m, 2H), 1.49 (d, J=6.8 Hz, 6H).

### Example 8

### Synthesis of Compound I-3

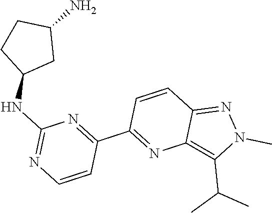 [SMILES: CC(C)c1c2nc(-c3ccnc(N[C@H]4CC[C@H](N)C4)n3)ccc2nn1C]

Overall Reaction Scheme:

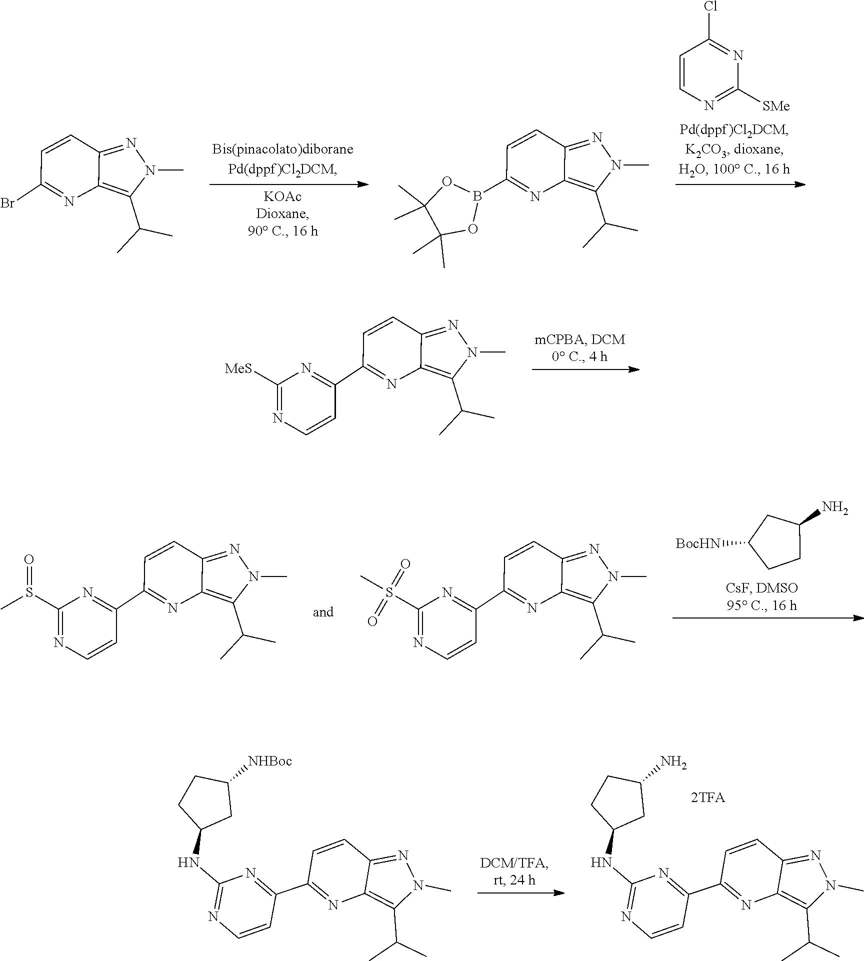

### Synthesis of 3-isopropyl-2-methyl-5-(4,4,5,5-tetramethyl-1,3,2-dioxaborolan-2-yl) pyrazolo[4,3-b]pyridine

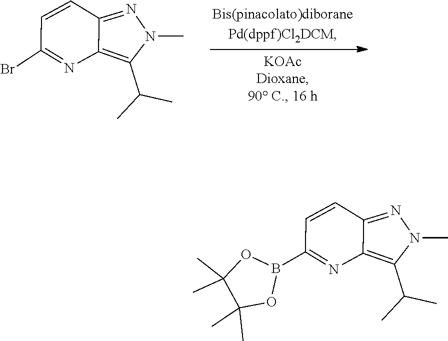

To a stirred solution of 5-bromo-2methyl-3-propylpyrazolo[4,3-b]pyridine (45 mg, 0.18 mmol, 1.0 eq) and bis(pinacolato)diborane (56 mg, 0.22 mmol, 1.2 eq) in 1,4-dioxane (8 mL) in a microwave vial was added potassium acetate (44 mg, 0.45 mmol, 2.5 eq), Pd(dppf)Cl₂·DCM (8 mg, 0.01 mmol, 0.05 eq). Then, the vial was sealed with the cap and degassed for 5 min with argon. The reaction mixture was stirred at 90° C. for 16 h. After completion of reaction by TLC, reaction mixture was diluted with water and ethyl acetate, resulting slurry was filtered through Celite® (i.e., diatomaceous earth). The organic layer was separated, washed with brine solution, dried over sodium sulfate and concentrated to provide crude product, 3-isopropyl-2-methyl-5-(4,4,5,5-tetramethyl-1,3,2-dioxaborolan-2-yl)pyrazolo[4,3-b]pyridine as brown oil (54 mg, Yield: 100%).

### Synthesis of 3-isopropyl-2-methyl-5-[2-(methylthio)pyrimidin-4-yl]pyrazolo[4,3-b]pyridine

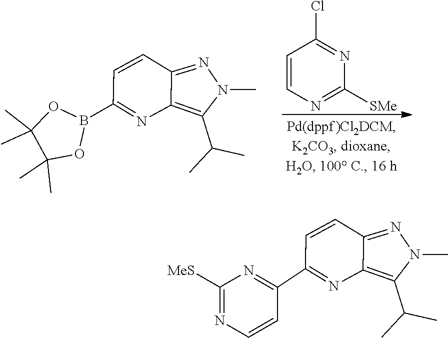

To a stirred solution of 3-isopropyl-2-methyl-5-(4,4,5,5-tetramethyl-1,3,2-dioxaborolan-2-yl)pyrazolo[4,3-b]pyridine (54 mg, 0.18 mmol, 1 eq) in 1,4-dioxane and water (3:1) (8 mL) in a microwave vial was added 4-chloro-2-(methylthio)pyrimidine (43 mg, 0.27 mmol, 1.5 eq), K₂CO₃ (62 mg, 0.45 mmol, 2.5 eq), Pd(dppf)Cl₂DCM (8 mg, 0.01 mmol, 0.05 eq) and degassed for 5 mins with argon. Then the reaction mixture was stirred at 100° C. for 16 h. The reaction mixture was diluted with water and ethyl acetate and the resulting slurry was filtered through Celite® (i.e., diatomaceous earth). The organic layer was separated, washed with brine solution, dried over sodium sulfate and concentrated to provide crude product which was purified by Combiflash Chromatography (4 g column) to afford 3-isopropyl-2-methyl-5-[2-(methylthio)pyrimidin-4-yl]pyrazolo[4,3-b]pyridine as a yellow solid (10 mg, yield: 18%). TLC system: Hexane: EtOAc (2:1), R_{f} value: ˜0.3;

### Synthesis of mixture of 3-isopropyl-2-methyl-5-(2-methylsulfinylpyrimidin-4-yl)pyrazolo[4,3-b]pyridine and 3-isopropyl-2-methyl-5-(2-methylsulfonylpyrimidin-4-yl)pyrazolo[4,3-b]pyridine

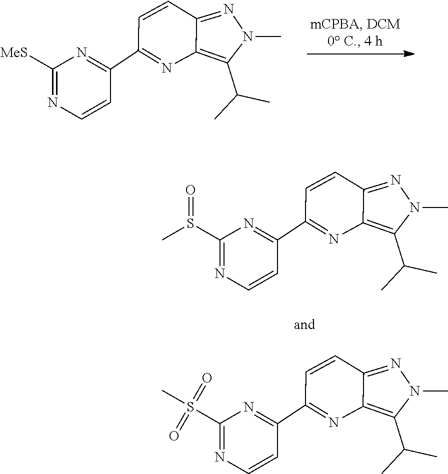

To a stirred solution of 3-isopropyl-2-methyl-5-[2-(methylthio)pyrimidin-4-yl]pyrazolo[4,3-b]pyridine (10 mg, 0.033 mmol, 1.0 eq) in DCM (4 ml) cooled to 0° C. and added 3-chloroperbenzoic acid (mCPBA) (purity, 77%) (10 mg, 0.043 mmol, 1.3 eq). The reaction mixture was stirred at 0° C. for 4 h. After completion of reaction by TLC, reaction mixture was quenched with sat. aq. NaHCO₃ solution (10 mL) and extracted with ethyl acetate. The combined organic layers were washed with brine solution, dried over sodium sulfate and concentrated to afford crude mixture of 3-isopropyl-2-methyl-5-(2-methylsulfinylpyrimidin-4-yl)pyrazolo[4,3-b]pyridine and 3-isopropyl-2-methyl-5-(2-methylsulfonylpyrimidin-4-yl)pyrazolo[4,3-b]pyridine as a yellow solid (10 mg, yield: 100%). TLC system: EtOAc (100%) R_{f} value: ˜0.01 and 0.2.

### Synthesis of tert-butyl (1S, 3S)-[3-[4-[3-isopropyl-2-methylpyrazolo[4,3-b]pyridin-5-yl]pyrimidin-2-yl]aminocyclopentan-1-yl]aminocarboxylate

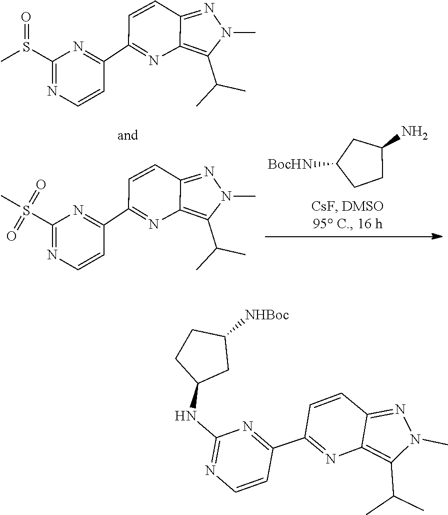

To a stirred solution of mixture of 3-isopropyl-2-methyl-5-(2-methylsulfinylpyrimidin-4-yl)pyrazolo[4,3-b]pyridine and 3-isopropyl-2-methyl-5-(2-methylsulfonylpyrimidin-4-yl)pyrazolo[4,3-b]pyridine (10 mg, 0.033 mmol, 1 eq) in DMSO (5 mL) at room temperature was added (1S,3S)-3-amino-1-(BOC-amino)cyclopentane (10 mg, 0.05 mmol, 1.5 eq) and cesium fluoride (8 mg, 0.05 mmol, 1.5 eq). Then the reaction mixture was stirred at 95° C. for 16 h. After completion of reaction by TLC, the reaction mixture was cooled to room temperature and extracted with ethyl acetate. The organic layer was washed with brine solution, dried over anhydrous sodium sulfate and then concentrated to provide product which was purified by Combiflash Chromatography (4 g column) to afford tert-butyl (1S, 3S)-[3-[4-[3-isopropyl-2-methylpyrazolo[4,3-b]pyridin-5-yl]pyrimidin-2-yl]aminocyclopentan-1-yl]aminocarboxylate as yellow solid (10 mg, 67%). TLC system: Hexane: EtOAc (1:1), R_{f} value: ˜0.2.

### Synthesis of (1S, 3S)-[3-[4-[3-isopropyl-2-methylpyrazolo[4,3-b]pyridin-5-yl]pyrimidin-2-yl]aminocyclopentan-1-yl]-1,3-diamine Trifluoroacetate

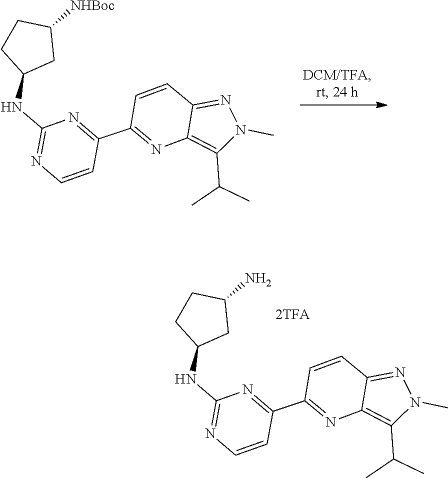 [SMILES: CC(C)c1c2nc(-c3ccnc(N[C@H]4CC[C@H](NC(=O)OC(C)(C)C)C4)n3)ccc2nn1C ; CC(C)c1c2nc(-c3ccnc(N[C@H]4CC[C@H](N)C4)n3)ccc2nn1C]

To a stirred solution of tert-butyl (1S, 3S)-[3-[4-[3-isopropyl-2-methylpyrazolo[4,3-b]pyridin-5-yl]pyrimidin-2-yl]aminocyclopentan-1-yl]aminocarboxylate (10 mg, 0.022 mmol, 1 eq) in DCM (4 mL) at room temperature was added trifluoroacetic acid (TFA) (2 ml) and the reaction mixture was stirred at room temperature for 24 h. After completion of reaction by TLC, the solvent was removed in vacuum. Then diethyl ether was added to the reaction and a lot of yellow solid will appear in the reaction. The mixture was centrifuged, washed over diethyl ether and dried at room temperature to provide the product, (1S, 3S)-[3-[4-[3-isopropyl-2-methylpyrazolo[4,3-b]pyridin-5-yl]pyrimidin-2-yl]aminocyclopentan-1-yl]-1,3-diamine Trifluoroacetate as yellow solid (5 mg, 38%). ¹HNMR (400 MHz, MeOD-d4) δ 8.53 (d, J=8.9 Hz, 1H), 8.40 (d, J=5.6 Hz, 1H), 8.05 (d, J=8.9 Hz, 1H), 7.86 (J=5.6 Hz, 1H), 4.68 (s, broad, 1H), 4.07 (s, 3H), 3.87-3.81 (m, 1H), 3.64-3.57 (m, 1H), 2.41-2.33 (m, 2H), 2.23-2.19 (m, 2H), 1.85-1.72 (m, 2H), 1.54 (d, J=7.0 Hz, 6H); HRMS data: HRMS (ESI) m/z [M+H]+ calcd for C₁₉H₂₅N₇, 352.2244, found: 352.2217

### Example 9

### Synthesis of Compound I-9

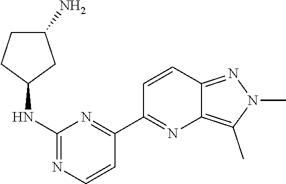 [SMILES: Cc1c2nc(-c3ccnc(N[C@H]4CC[C@H](N)C4)n3)ccc2nn1C]

Overall Reaction Scheme

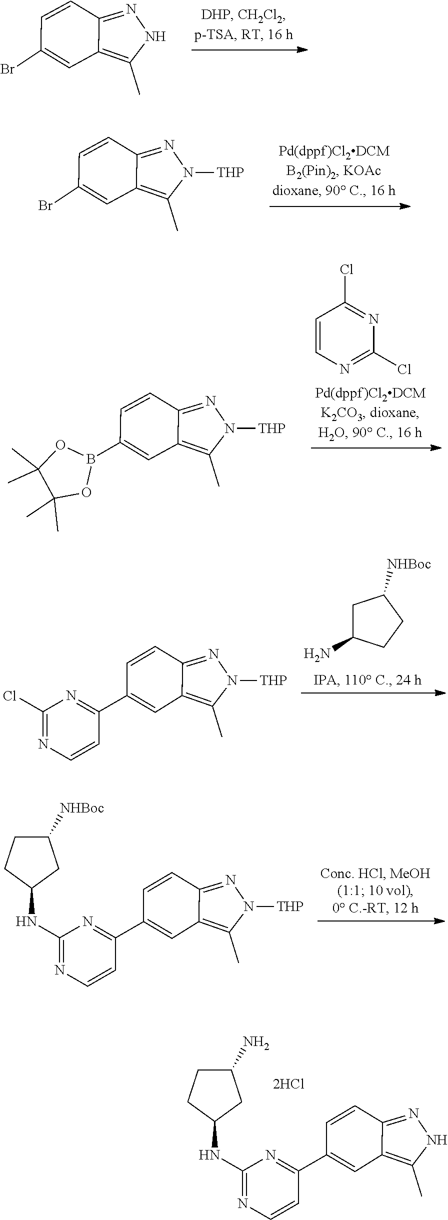

### Synthesis of 5-bromo-3-methyl-2-(tetrahydro-2H-pyran-2-yl)-2H-indazole

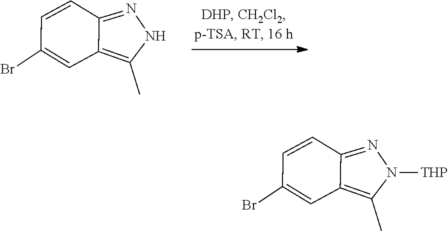

To a stirred solution of 5-bromo-3-methyl-2H-indazole (500 mg, 2.36 mmol, 1.0 eq) in DCM (5 mL) at room temperature was added dihydropyran (0.43 mL, 4.73 mmol, 2.0 eq), p-TSA (22 mg, 0.04 mmol, 0.05 eq) and stirred for 16 h. After completion of reaction by TLC, diluted with cold water (30 mL) and DCM (2×30 mL). Organic layer was dried over sodium sulfate, filtered, concentrated and purified by silica gel column (60-120 mesh, eluent: 10% EtOAc/petroleum ether) to afford 5-bromo-3-methyl-2-(tetrahydro-2H-pyran-2-yl)-2H-indazole as a yellow liquid (550 mg, Yield: 79%). TLC system: EtOAc/petroleum ether (40:60), R_{f} value: ˜0.8.

¹H NMR (400 MHz, CDCl₃) δ 7.78 (dd, J=0.8 & 2 Hz, 1H), 7.46-7.39 (m, 2H), 5.59 (dd, J=10 & 2.8 Hz, 1H), 4.07-4.04 (m, 1H), 3.75-3.69 (m, 1H), 2.56-2.50 (m, 4H), 2.15-2.12 (m, 1H), 2.05-2.00 (m, 1H), 1.77-1.71 (m, 2H), 1.65-1.62 (m, 1H).

### Synthesis of 3-methyl-2-(tetrahydro-2H-pyran-2-yl)-5-(4,4,5,5-tetramethyl-1,3,2-dioxaborolan-2-yl)-2H-indazole

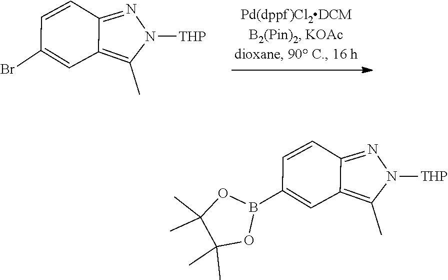

To a stirred solution of 5-bromo-3-methyl-2-(tetrahydro-2H-pyran-2-yl)-2H-indazole (550 mg, 1.86 mmol, 1.0 eq), bis(pinacolato)diborane (570 mg, 2.23 mmol, 1.2 eq) and potassium acetate (460 mg, 4.66 mmol, 2.5 eq), in 1,4-dioxane (11 mL) was degassed for 5 min and added Pd(dppf)Cl₂·DCM (76 mg, 0.093 mmol, 0.05 eq). The reaction mixture was stirred at 90° C. for 16 h. After completion of reaction by TLC, diluted with water (50 mL). and extracted with ethyl acetate (2×50 mL). Organic layer was dried over sodium sulfate and concentrated to afford 3-methyl-2-(tetrahydro-2H-pyran-2-yl)-5-(4,4,5,5-tetramethyl-1,3,2-dioxaborolan-2-yl)-2H-indazole (630 mg, Crude) as black semi solid. TLC system: EtOAc/petroleum ether (20:80), R_{f} value: ˜0.3. LC/MS Retention time=4.19 min, 343.2 [M+H]⁺. Material was taken forward to next step.

### Synthesis of 5-(2-chloropyrimidin-4-yl)-3-methyl-2-(tetrahydro-2H-pyran-2-yl)-2H-indazole

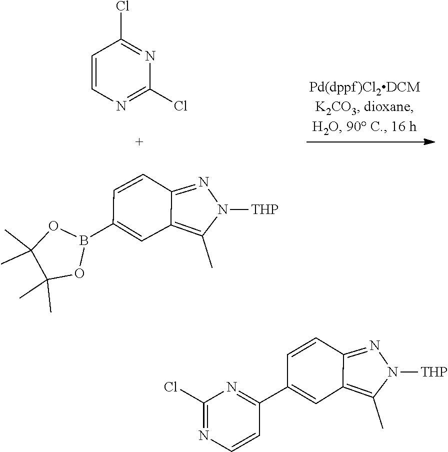

To a degassed stirred solution of 3-methyl-2-(tetrahydro-2H-pyran-2-yl)-5-(4,4,5,5-tetramethyl-1,3,2-dioxaborolan-2-yl)-2H-indazole (630 mg, 1.84 mmol, 1.0 eq) in 1,4-dioxane and water (3:1) (12 mL) was added 2,4-dichloropyrimidine (329 mg, 2.21 mmol, 1.2 eq) and K₂CO₃ (635 mg, 4.60 mmol, 2.5 eq). The reaction mixture was degassed for 5 mins and added Pd(dppf)Cl₂DCM (75 mg, 0.092 mmol, 0.05 eq). The reaction mixture was stirred at 90° C. for 16 h. After completion of reaction by TLC, diluted with water and extracted with ethyl acetate (2×50 mL). Combined organic layer was dried over sodium sulfate, concentrated and purified by silica gel column (60-120 mesh, eluent: 50% EtOAc/petroleum ether) to afford 5-(2-chloropyrimidin-4-yl)-3-methyl-2-(tetrahydro-2H-pyran-2-yl)-2H-indazole (550 mg, Semi-pure) as off-white solid. TLC system: EtOAc/petroleum ether (30:70), R_{f} value: ˜0.3; LC/MS Retention time=3.76 min, 329.1 [M+H]⁺; 69% purity. Material was taken forward to next step.

### Synthesis of tert-butyl ((1S,3S)-3-((4-(3-methyl-2-(tetrahydro-2H-pyran-2-yl)-2H-indazol-5-yl)pyrimidin-2-yl)amino)cyclopentyl)carbamate

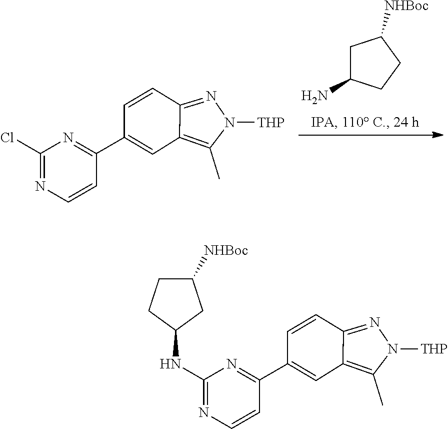

To a stirred solution of 5-(2-chloropyrimidin-4-yl)-3-methyl-2-(tetrahydro-2H-pyran-2-yl)-2H-indazole (550 mg, 1.67 mmol, 1 eq) in IPA (8 mL) was added tert-butyl ((1S,3S)-3-aminocyclopentyl)carbamate (335 mg, 2.01 mmol, 1.2 eq) and stirred at 110° C. for 24 h. TLC showed polar spot and RM was allowed to room temperature. The reaction mixture was evaporated and diluted with water (30 mL) and extracted with EtOAc (3×20 ml). Organic layer was dried over Na₂SO₄, filtered, evaporated and purified by silica gel column (60-120 mesh, eluent: 15% to 60% EtOAc/petroleum ether) to afford tert-butyl ((1S,3S)-3-((4-(3-methyl-2-(tetrahydro-2H-pyran-2-yl)-2H-indazol-5-yl)pyrimidin-2-yl)amino)cyclo pentyl)carbamate (240 mg, semi-pure) as off white solid. TLC system: EtOAc/petroleum ether (70:30), R_{f} value: ˜0.20; LC/MS Retention time=4.06 min, 493.3 [M+H]⁺; 58% purity. Material was taken forward to next step.

### Synthesis of (1S,3S)—N1-(4-(3-methyl-2H-indazol-5-yl)pyrimidin-2-yl)cyclopentane-1,3-diamine HCl salt

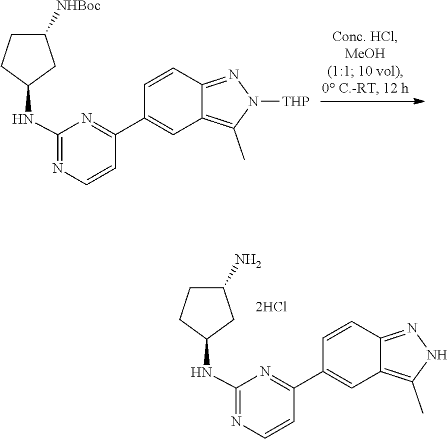

To a stirred solution of tert-butyl ((1S,3S)-3-((4-(3-methyl-2-(tetrahydro-2H-pyran-2-yl)-2H-indazol-5-yl)pyrimidin-2-yl)amino)cyclo pentyl)carbamate (170 mg, 0.29 mmol, 1 eq.) in MeOH (1 mL) at 0° C. was added Conc. HCl (1 mL) and stirred at room temperature for 12 h. After completion of starting material, volatiles removed, triturated with diethyl ether (2×5 mL) and purified by Prep-HPLC (ABC method). Ammonium salts were avoided by doing work-up and fractions were lyophilized by adding aq. HCl to afford (1S,3S)—N1-(4-(3-methyl-2H-indazol-5-yl)pyrimidin-2-yl)cyclopentane-1,3-diamine HCl salt (32 mg, Yield: 5% in 4 steps) as yellow solid. TLC system: MeOH/DCM (30:70), R_{f} value: ˜0.1; LC/MS Retention time=1.78 min, 309.2 [M+H]⁺.

¹H NMR (400 MHz, CD₃OD) δ 8.83 (s, 1H), 8.42 (d, J=6.4 Hz, 1H), 8.31 (d, J=6.8 Hz, 1H), 7.73 (d, J=6.8 Hz, 1H), 7.67 (d, J=8.8 Hz, 1H), 3.91-3.88 (m, 1H), 3.29-3.28 (m, 1H), 2.69 (s, 3H), 2.48-2.38 (m, 2H), 2.30 (t, J=6.8 Hz, 2H), 1.93-1.82 (m, 2H).

### Example 10

### Synthesis of Compound I-4

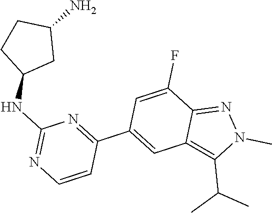 [SMILES: CC(C)c1c2cc(-c3ccnc(N[C@H]4CC[C@H](N)C4)n3)cc(F)c2nn1C]

Overall Reaction Scheme

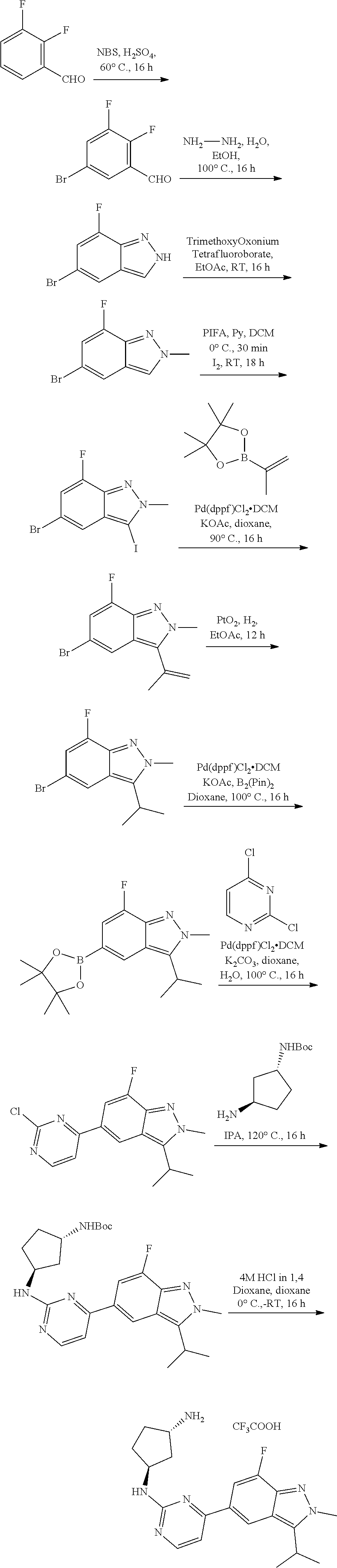

### Synthesis of 5-bromo-2,3-difluorobenzaldehyde

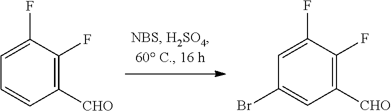 [SMILES: O=Cc1cccc(F)c1F ; O=Cc1cc(Br)cc(F)c1F]

To a stirred solution of 2,3-difluorobenzaldehyde (5 g, 35.2 mmol, 1.0 eq) in sulfuric acid (38 mL) at room temperature was added NBS (31.3 mL, 52.78 mmol, 1.5 eq) and stirred at 60° C. for 16 h. After completion of reaction by TLC, diluted with cold water (50 mL) and extracted with EtOAc (2×100 mL). Organic layer was washed with NaHCO₃ solution, dried over sodium sulfate and concentrated to provide 7 g material. The crude compound was purified by silica gel column (100-200 mesh, eluent: 2% EtOAc/hexane) to afford 5-bromo-2,3-difluorobenzaldehyde as yellow liquid (3.5 g, 45%). TLC system: EtOAc/petroleum ether (05:95), R_{f} value: ˜0.8.

¹H NMR (400 MHz, CDCl₃) δ 10.30 (s, 1H), 7.79-7.77 (m, 1H), 7.63-7.58 (m, 1H).

### Synthesis of 5-bromo-7-fluoro-2H-indazole

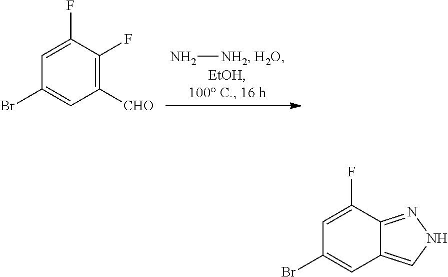

To a stirred solution of 5-bromo-2,3-difluorobenzaldehyde (2) (3.4 g, 15.5 mmol, 1.0 eq) in EtOH (34 mL, 10 Vol) at room temperature was added 85% hydrazine·hydrate (17 mL, 5 Vol) and stirred at 100° C. for 16 h. After completion of reaction by TLC, RM was evaporated, diluted with water (100 mL) and extracted with ethylacetate (3×50 mL). The organic layer was separated, dried over sodium sulfate and concentrated to provide 4.1 g material. The crude compound was purified by silica gel column (60-120 mesh, eluent: 25% to 35% EtOAc/hexane) to afford 5-bromo-7-fluoro-2H-indazole as off-white solid (1.7 g, 51%). TLC system: EtOAc/petroleum ether (20:80), R_{f} value: ˜0.2. LC/MS Retention time=3.10 min, 214.9 [M+H]+.

### Synthesis of 5-bromo-7-fluoro-2-methyl-2H-indazole

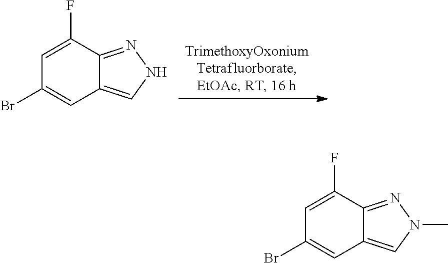

To a stirred solution of 5-bromo-7-fluoro-2H-indazole (1.7 g, 7.94 mmol, 1.0 eq) in EtOAc (54 mL, 30 Vol) at room temperature was added Trimethyloxoniumtetrafluoroborate (7.05 g, 47.7 mmol, 6.0 eq) and continued stirring for 16 h. After completion of reaction by TLC, the reaction mixture was evaporated and purified by silica gel column (60-120 mesh, eluent: 15% to 18% EtOAc/hexane) to afford 5-bromo-7-fluoro-2-methyl-2H-indazole (750 mg, 42%) as off white solid. TLC system: EtOAc/petroleum ether (20:80), R_{f} value: ˜0.5.

¹H NMR (400 MHz, CDCl₃) δ 7.91 (d, J=2.8 Hz, 1H), 7.61 (d, J=1.6 Hz, 1H), 7.08 (dd, J=10.4 & 1.6 Hz, 1H), 4.26 (s, 3H). confirmed by nOe.

### Synthesis of 5-bromo-7-fluoro-3-iodo-2-methyl-2H-indazole

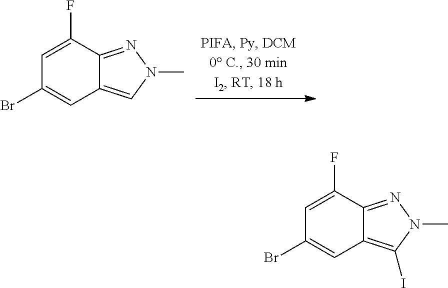

To a stirred solution of 5-bromo-7-fluoro-2-methyl-2H-indazole (0.75 g, 3.28 mmol, 1.0 eq) in DCM (15 mL, 20 Vol) at 0° C., was added pyridine (1.7 g, 4.93 mmol, 1.5 eq) and PIFA (1.7 g, 3.95 mmol, 1.2 eq). Reaction mixture was stirred for 30 min at 0° C., later added Iodine (0.5 g, 3.95 mmol, 1.2 eq) and allowed to stirred at room temperature for 18 h. After completion of reaction by TLC, the reaction mixture was diluted with sat NaHCO₃ (50 mL) and extracted with DCM (2×20 mL). The organic layer was separated, dried over sodium sulfate, and concentrated to provided crude product. Purified by silica gel column (60-120 mesh, eluent:5% EtOAc/hexane) to afford 5-bromo-7-fluoro-3-iodo-2-methyl-2H-indazole (700 mg, 60%) as a yellow liquid. TLC system: EtOAc/petroleum ether (20:80), R_{f} value: ˜0.8.

¹H NMR (400 MHz, CDCl₃) δ 7.38 (d, J=1.6 Hz, 1H), 7.10 (dd, J=10.4 & 1.6 Hz, 1H), 4.27 (s, 3H).

### Synthesis of 5-bromo-7-fluoro-2-methyl-3-(prop-1-en-2-yl)-2H-indazole

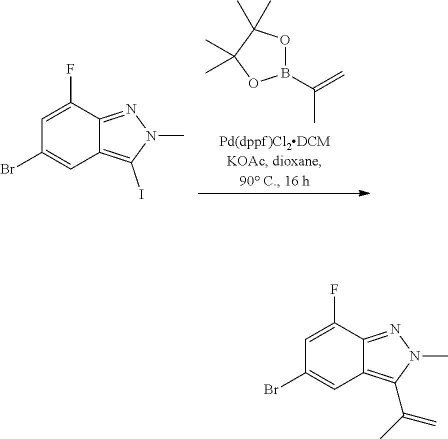

To a stirred solution of 5-bromo-7-fluoro-3-iodo-2-methyl-2H-indazole (700 mg, 1.98 mmol, 1.0 eq) in 1,4-dioxane (14 mL) at room temperature was added 4,4,5,5-tetramethyl-2-(prop-1-en-2-yl)-1,3,2-dioxaborolane (398 mg, 2.37 mmol, 1.2 eq) and potassium carbonate (682 mg, 4.94 mmol, 2.5 eq), degassed for 5 min. Later added Pd(dppf)Cl₂·DCM (80 mg, 0.098 mmol, 0.05 eq). The reaction mixture was stirred at 90° C. for 16 h. After completion of reaction by TLC. The volatiles evaporated, residue was diluted with water (80 mL) and extracted with ethyl acetate (2×50 mL). The organic layer was separated, washed with brine solution (30 mL), dried over sodium sulfate and concentrated. The crude compound was purified by silica gel column (60-120 mesh, eluent:10% to 12% EtOAc/hexane) to afford 5-bromo-7-fluoro-2-methyl-3-(prop-1-en-2-yl)-2H-indazole as a yellow liquid (450 mg, 85%). TLC system: EtOAc/petroleum ether (20:80), R_{f} value: ˜0.50. LC/MS Retention time=3.99 min, 269.0 [M+H]⁺.

¹H NMR (400 MHz, CDCl₃) δ 7.54 (s, 1H), 7.04 (d, J=10 Hz, 1H), 5.63 (s, 1H), 5.28 (s, 1H), 4.16 (s, 3H), 2.22 (s, 3H).

### Synthesis of 5-bromo-7-fluoro-3-isopropyl-2-methyl-2H-indazole

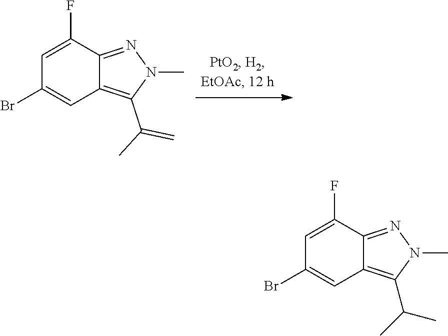

To a stirred solution of 5-bromo-7-fluoro-2-methyl-3-(prop-1-en-2-yl)-2H-indazole (400 mg, 1.49 mmol, 1.0 eq) in EtOAc (8 mL, 20 vol) was added PtO₂ (11.3 mg, 0.036 mmol, 0.05 eq) and stirred at room temperature for 7 h under H₂ atmosphere After completion of reaction by TLC, the reaction mixture was filtered through Celite® (i.e., diatomaceous earth), washed with EtOAc. Organic layer evaporated and purified by silica gel column (100-200 mesh, eluent: 50% EtOAc/hexane) to afford 5-bromo-7-fluoro-3-isopropyl-2-methyl-2H-indazole as brown solid (360 mg, 70%). TLC system: EtOAc/petroleum ether (50:50), R_{f} value: ˜0.25; LC/MS Retention time=3.87 min, 271.0 [M+H]⁺.

¹H NMR (400 MHz, CDCl₃) δ 7.70 (d, J=1.6 Hz, 1H), 7.02 (dd, J=10.4 & 1.6 Hz, 1H), 4.16 (s, 3H), 3.45-3.38 (m, 1H), 1.51 (d, J=6.8 Hz, 6H).

### Synthesis of 7-fluoro-3-isopropyl-2-methyl-5-(4,4,5,5-tetramethyl-1,3,2-dioxaborolan-2-yl)-2H-indazole

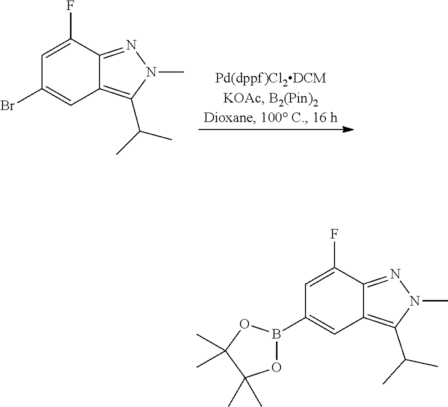

To a stirred solution of 5-bromo-7-fluoro-3-isopropyl-2-methyl-2H-indazole (300 mg, 1.11 mmol, 1.0 eq) in 1,4-dioxane (6 mL) at room temperature was added bis(pinacolato)diborane (335 mg, 1.33 mmol, 1.2 eq) and potassium acetate (272 mg, 2.77 mmol, 2.5 eq), degassed for 5 min followed by addition of Pd(dppf)Cl₂·DCM (45 mg, 0.055 mmol, 0.05 eq). The reaction mixture was stirred at 100° C. for 16 h. After completion of reaction by TLC. Volatiles removed under vacuum, diluted with water (50 mL) and extracted with ethyl acetate (3×30 mL). The organic layer was separated, washed with brine solution (30 mL), dried over sodium sulfate, and concentrated to afford 7-fluoro-3-isopropyl-2-methyl-5-(4,4,5,5-tetramethyl-1,3,2-dioxaborolan-2-yl)-2H-indazole as a black semi solid (350 mg, crude). TLC system: EtOAc/petroleum ether (20:80), R_{f} value: ˜0.45. Cr: LC/MS shows 44% boronic ester and 16.3% Boronic acid m/z. LC/MS Retention time=5.39 min, 319.2 [M+H]⁺. Crude material used as such in next step.

### Synthesis of 5-(2-chloropyrimidin-4-yl)-7-fluoro-3-isopropyl-2-methyl-2H-indazole

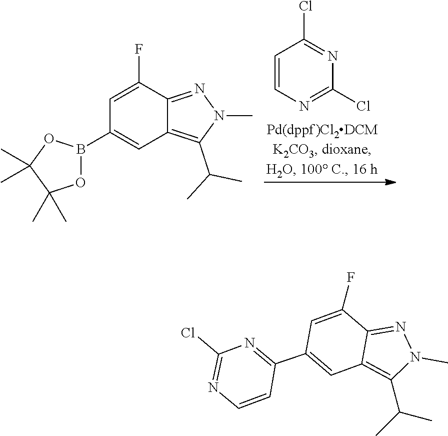

To a stirred solution of 7-fluoro-3-isopropyl-2-methyl-5-(4,4,5,5-tetramethyl-1,3,2-dioxaborolan-2-yl)-2H-indazole (350 mg, 1.10 mmol, 1 eq) in 1,4-dioxane and water (3:1) (6 mL) was added 4-chloro-2-(methylthio)pyrimidine (208 mg, 1.43 mmol, 1.3 eq) and K₂CO₃ (380 mg, 2.75 mmol, 2.5 eq). The reaction mixture was degassed for 5 mins and added Pd(dppf)Cl₂DCM (45 mg, 0.055 mmol, 0.05 eq). The reaction mixture was stirred at 100° C. for 16 h. After completion of reaction by TLC, diluted with water and extracted with ethyl acetate (3×20 mL) The organic layer dried over Na2SO4, evaporated and purified silica gel column (100-200 mesh, eluent: 25% to 30% EtOAc/hexanes) to afford 5-(2-chloropyrimidin-4-yl)-7-fluoro-3-isopropyl-2-methyl-2H-indazole (180 mg, 54%) as yellow solid. TLC system: EtOAc/hexanes (50:50), R_{f} value: ˜0.25. LC/MS Retention time=3.53 min, 305.0 [M+H]⁺.

¹H NMR (400 MHz, CDCl₃) δ 8.61 (d, J=5.6 Hz, 1H), 8.41 (s, 1H), 7.61-7.57 (m, 2H), 4.20 (s, 3H), 3.56-3.48 (m, 1H), 1.58 (d, J=7.2 Hz, 6H).

### Synthesis of tert-butyl ((1S,3S)-3-((4-(7-fluoro-3-isopropyl-2-methyl-2H-indazol-5-yl)pyrimidin-2-yl)amino)cyclopentyl)carbamate

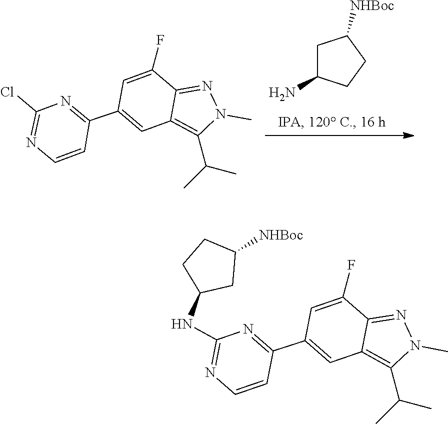 [SMILES: CC(C)c1c2cc(-c3ccnc(Cl)n3)cc(F)c2nn1C ; CC(C)(C)OC(=O)N[C@@H]1CC[C@@H](N)C1 ; CC(C)c1c2cc(-c3ccnc(N[C@H]4CC[C@H](NC(=O)OC(C)(C)C)C4)n3)cc(F)c2nn1C]

To a stirred solution of 5-(2-chloropyrimidin-4-yl)-7-fluoro-3-isopropyl-2-methyl-2H-indazole (150 mg, 0.493 mmol, 1 eq) in isopropyl alcohol (2.5 mL) was added tert-butyl ((1S,3S)-3-aminocyclopentyl)carbamate (120 mg, 0.64 mmol, 1.3 eq) and heated at 120° C. for 16 h. TLC showed polar spot and the reaction mixture was allowed to room temperature. The reaction mixture was evaporated, diluted with water (20 mL) and extracted with EtOAc (2×20 mL). Organic layer was dried over Na₂SO₄, filtered, evaporated and purified by silica gel column (eluent in 40% EtOAc/hexane) to afford tert-butyl tert-butyl ((1S,3S)-3-((4-(7-fluoro-3-isopropyl-2-methyl-2H-indazol-5-yl)pyrimidin-2-yl)amino)cyclopentyl)carbamate (130 mg, 56%) as yellow solid. TLC system: MeOH/DCM (10:90), R_{f} value: ˜0.30; LC/MS Retention time=3.50 min, 469.2 [M+H]⁺.

### Synthesis of (1S,3S)—N-(4-(7-fluoro-3-isopropyl-2-methyl-2H-indazol-5-yl)pyrimidin-2-yl)cyclopentane-1,3-diamine TFA salt

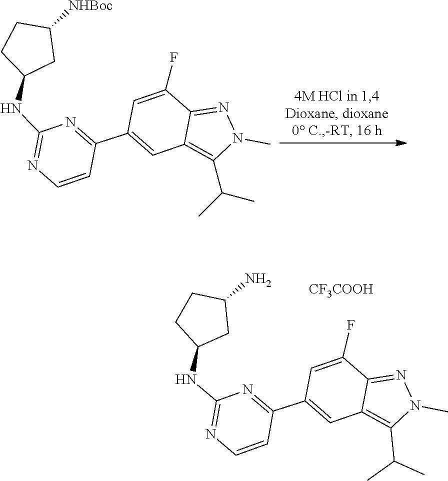 [SMILES: CC(C)c1c2cc(-c3ccnc(N[C@H]4CC[C@H](NC(=O)OC(C)(C)C)C4)n3)cc(F)c2nn1C ; "4M HCl in 1,4Dioxane, dioxane0° C.,-RT, 16 h" ; CC(C)c1c2cc(-c3ccnc(N[C@H]4CC[C@H](N)C4)n3)cc(F)c2nn1C ; "CF3COOH"]

A solution of tert-butyl ((1S,3S)-3-((4-(3-isopropyl-1-methyl-1H-pyrazolo[3,4-b]pyridin-5-yl)pyrimidin-2-yl)amino)cyclopentyl)carbamate (130 mg, 0.277 mmol, 1 eq.) in 1,4-Dioxane (1.3 mL) at 0° C., was added 4M HCl in 1,4-Dioxane (0.3 mL) and stirred for 16 h at room temperature. After completion of starting material, reaction mixture was concentrated and purified by prep HPLC (eluent in 32% acetonitrile and 0.1% TFA in water) to afforded (1S,3S)—N1-(4-(7-fluoro-3-isopropyl-2-methyl-2H-indazol-5-yl)pyrimidin-2-yl)cyclopentane-1,3-diamine TFA salt (40 mg, 39%) as yellow solid. TLC system: MeOH/DCM (10:90), R_{f} value: ˜0.1; LC/MS Retention time=2.67 min, 369.3 [M+H]⁺.

¹H NMR (400 MHz, CD₃OD) δ 8.55 (s, 1H), 8.29 (d, J=6.4 Hz, 1H), 7.85 (d, J=13.2 Hz, 1H), 7.47 (d, J=5.6 Hz, 1H), 4.71-4.68 (m, 1H), 4.20 (s, 3H), 3.86-3.83 (m, 1H), 3.69-3.61 (m, 1H), 2.41-2.35 (m, 2H), 2.25-2.19 (m, 2H), 1.88-1.82 (m, 1H), 1.80-1.75 (m, 1H), 1.59 (d, J=7.2 Hz, 6H).

### Example 11

### Synthesis of Compound I-10

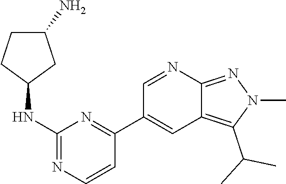 [SMILES: CC(C)c1c2cc(-c3ccnc(N[C@H]4CC[C@H](N)C4)n3)cnc2nn1C]

Overall Reaction Scheme:

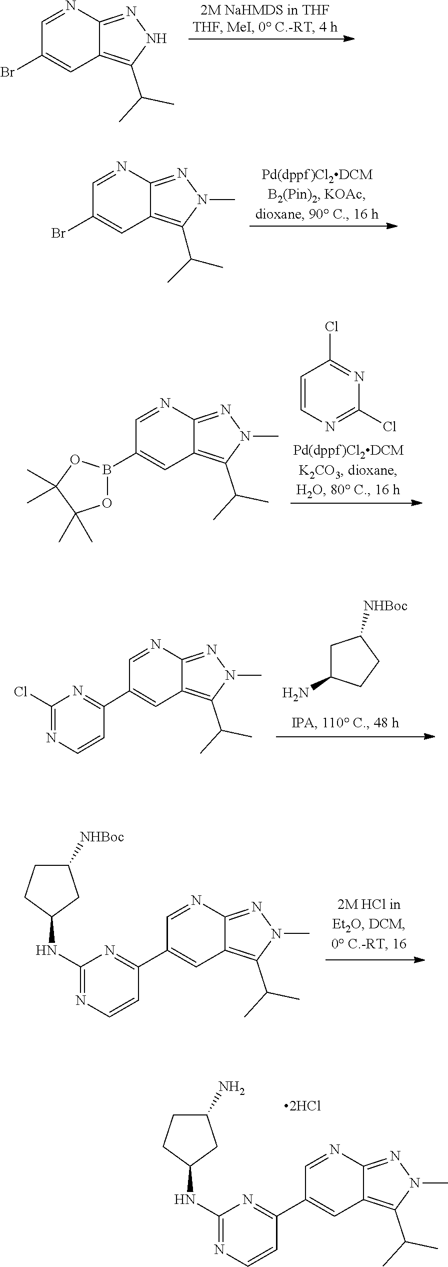

### Synthesis of 5-bromo-3-isopropyl-2-methyl-2H-pyrazolo[3,4-b]pyridine

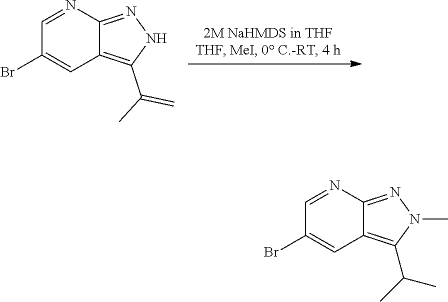

To a stirred solution of 5-bromo-3-isopropyl-2H-pyrazolo[3,4-b]pyridine (0.8 g, 3.33 mmol, 1.0 eq) in THF (24 mL, 30 Vol) at 0° C., was added 2M NaHMDS in THF (2.4 mL, 4.99 mmol, 1.5 eq) followed by Mel (0.8 mL, 9.99 mmol, 3 eq.) and stirred at room temperature for 4 h. After completion of reaction by TLC, diluted with cold water (100 mL) and extracted with EtOAc (2×80 mL). The organic layer was separated, dried over sodium sulfate and concentrated to afford 5-bromo-3-isopropyl-2-methyl-2H-pyrazolo[3,4-b]pyridine (600 mg, Yield: 71%) as yellow solid. TLC system: EtOAc/petroleum ether (1:1), R_{f} value: ˜0.4; ¹H NMR (400 MHz, CDCl₃) δ 8.61 (d, J=2.4 Hz, 1H), 8.25 (d, J=2.0 Hz, 1H), 4.13 (s, 3H), 3.43-3.39 (m, 1H), 1.49 (d, J=7.2 Hz, 6H).

### Synthesis of 3-isopropyl-2-methyl-5-(4,4,5,5-tetramethyl-1,3,2-dioxaborolan-2-yl)-2H-pyrazolo[3,4-b]pyridine

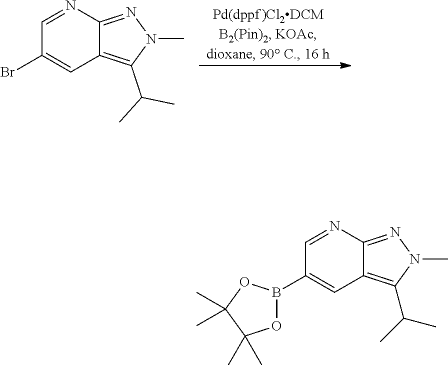

To a stirred solution of 5-bromo-3-isopropyl-2-methyl-2H-pyrazolo[3,4-b]pyridine (600 mg, 2.36 mmol, 1.0 eq) in 1,4-dioxane (12 mL) was added bis(pinacolato)diborane (722 mg, 2.83 mmol, 1.2 eq), potassium acetate (581 mg, 5.90 mmol, 2.5 eq) and degassed for 5 min. Later added Pd(dppf)Cl₂·DCM (96 mg, 0.12 mmol, 0.05 eq) and stirred at 90° C. for 16 h. After completion of reaction by TLC. Volatiles removed under vacuum, diluted with water (70 mL) and extracted with ethyl acetate (3×30 mL). The organic layer was separated, washed with brine solution (30 mL), dried over sodium sulfate and concentrated to provide 3-isopropyl-2-methyl-5-(4,4,5,5-tetramethyl-1,3,2-dioxaborolan-2-yl)-2H-pyrazolo[3,4-b]pyridine (700 mg, crude) as gummy mass. TLC system: EtOAc (100%), R_{f} value: ˜0.1; Cr: LC/MS: 55% boronic acid m/z, 220.1 [M+H]⁺.

### Synthesis of 5-(2-chloropyrimidin-4-yl)-3-isopropyl-2-methyl-2H-pyrazolo[3,4-b]pyridine

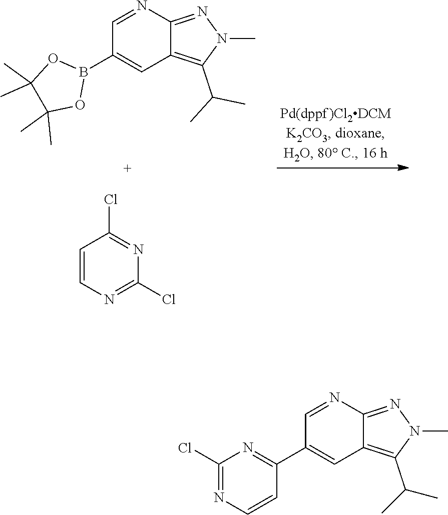

To a stirred solution of 3-isopropyl-2-methyl-5-(4,4,5,5-tetramethyl-1,3,2-dioxaborolan-2-yl)-2H-pyrazolo[3,4-b]pyridine (700 mg, 2.32 mmol, 1 eq) in 1,4-dioxane and water (3:1) (14 mL) was added 4-chloro-2-(methylthio)pyrimidine (572 mg, 2.78 mmol, 1.2 eq), K₂CO₃ (1.1 g, 5.80 mmol, 2.5 eq) and degassed for 5 min. Later added Pd(dppf)Cl₂DCM (131 mg, 0.12 mmol, 0.05 eq) and stirred at 80° C. for 16 h. After completion of reaction by TLC, the reaction mixture was evaporated, diluted with water (80 mL) and extracted with Ethyl acetate (2×70 mL). Organic layer was separated, dried over sodium sulfate and concentrated to provided 2.6 g crude which was purified by silica gel column (100-200 mesh, eluent: 80% EtOAc/petroleum ether) to afford 5-(2-chloropyrimidin-4-yl)-3-isopropyl-2-methyl-2H-pyrazolo[3,4-b]pyridine (180 mg, Yield: 26%) as gummy liquid. TLC system: EtOAc (100%), R_{f} value: ˜0.2;

¹H NMR (400 MHz, CDCl₃) δ 9.28 (d, J=2.4 Hz, 1H), 9.04 (d, J=2.4 Hz, 1H), 8.69 (d, J=5.2 Hz, 1H), 7.75 (d, J=5.2 Hz, 1H), 4.26 (s, 3H), 3.58-3.53 (m, 1H), 1.63 (d, J=6.8 Hz, 6H).

### Synthesis of tert-butyl ((1S,3S)-3-((4-(3-isopropyl-2-methyl-2H-pyrazolo[3,4-b]pyridin-5-yl)pyrimidin-2-yl)amino)cyclopentyl)carbamate

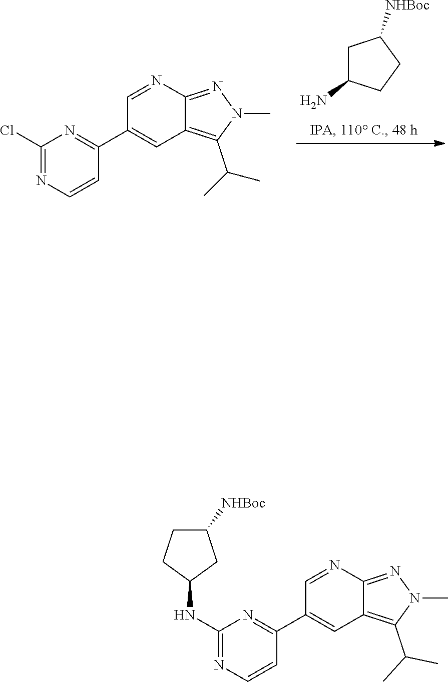 [SMILES: CC(C)c1c2cc(-c3ccnc(Cl)n3)cnc2nn1C ; CC(C)(C)OC(=O)N[C@@H]1CC[C@@H](N)C1 ; CC(C)c1c2cc(-c3ccnc(N[C@H]4CC[C@H](NC(=O)OC(C)(C)C)C4)n3)cnc2nn1C]

To a stirred solution of 5-(2-chloropyrimidin-4-yl)-3-isopropyl-2-methyl-2H-pyrazolo[3,4-b]pyridine (180 mg, 0.63 mmol, 1 eq) in IPA (1.8 mL) was added tert-butyl ((1S,3S)-3-aminocyclopentyl)carbamate (163 mg, 0.82 mmol, 1.3 eq) and stirred at 110° C. for 48 h. TLC showed new polar spot and traces of starting material. Volatiles removed under vacuum, diluted with water (20 mL) and extracted with EtOAc (2×20 ml). Organic layer was dried over Na₂SO₄, filtered, concentrated and purified by silica gel column (100-200 mesh, eluent: 70-80% EtOAc/petroleum ether) to afford tert-butyl ((1S,3S)-3-((4-(3-isopropyl-2-methyl-2H-pyrazolo[3,4-b]pyridin-5-yl)pyrimidin-2-yl)amino)cyclopentyl)carbamate (200 mg, 70%) as yellow gummy solid. TLC system: MeOH/DCM (5:95), R_{f} value: ˜0.4; LC/MS: Retention time=3.49 min, 452.2 [M+H]⁺.

¹H NMR (400 MHz, CDCl₃): δ 9.31 (brs, 1H), 8.83 (s, 1H), 8.38 (d, J=5.2 Hz, 1H), 7.06 (d, J=5.2 Hz, 1H), 5.26 (d, J=6.8 Hz, 1H), 4.62 (brs, 1H), 4.55-4.50 (m, 1H), 4.24-4.18 (m, 4H), 3.57-3.50 (m, 1H), 2.38-2.25 (m, 2H), 2.08-1.99 (m, 2H), 1.61 (d, J=7.2 Hz, 6H), 1.58-1.52 (m, 2H), 1.49 (s, 9H).

### Synthesis of (1S,3S)—N1-(4-(3-isopropyl-2-methyl-2H-pyrazolo[3,4-b]pyridin-5-yl)pyrimidin-2-yl)cyclopentane-1,3-diamine HCl salt

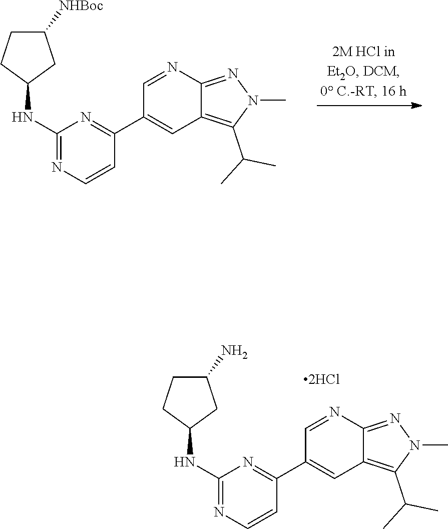 [SMILES: CC(C)c1c2cc(-c3ccnc(N[C@H]4CC[C@H](NC(=O)OC(C)(C)C)C4)n3)cnc2nn1C ; CC(C)c1c2cc(-c3ccnc(N[C@H]4CC[C@H](N)C4)n3)cnc2nn1C]

To a stirred solution of tert-butyl ((1S,3S)-3-((4-(3-isopropyl-2-methyl-2H-pyrazolo[3,4-b]pyridin-5-yl)pyrimidin-2-yl)amino)cyclopentyl)carbamate (6) (200 mg, 0.44 mmol, 1 eq.) in DCM (2 mL) at 0° C., was added 2M HCl in Et₂O (1 mL) and stirred for 16 h at room temperature. After completion of starting material, volatiles removed under vacuum, triturated with diethyl ether (2×3 mL) and lyophilized to afford ((1S,3S)—N1-(4-(3-isopropyl-2-methyl-2H-pyrazolo[3,4-b]pyridin-5-yl)pyrimidin-2-yl)cyclopentane-1,3-diamine HCl salt (94 mg, 50%) as brown solid. TLC system: MeOH/DCM (10:90), R_{f} value: ˜0.1; LC/MS Retention time=2.29 min, 352.2 [M+H]⁺.

¹H NMR (400 MHz, CD₃OD) δ 9.79-9.74 (m, 2H), 8.48 (d, J=6.8 Hz, 1H), 7.77 (d, J=6.4 Hz, 1H), 5.11-5.05 (br, 1H), 4.34 (s, 3H), 3.94-3.87 (m, 1H), 3.84-3.77 (m, 1H), 2.49-2.36 (m, 2H), 2.33-2.29 (m, 2H), 1.94-1.81 (m, 2H), 1.66 (d, J=6.8 Hz, 6H).

### Example 12

### Synthesis of Compound I-11

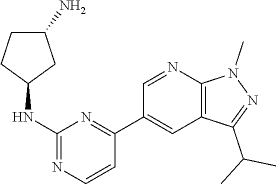 [SMILES: CC(C)c1nn(C)c2ncc(-c3ccnc(N[C@H]4CC[C@H](N)C4)n3)cc12]

Overall Reaction Scheme:

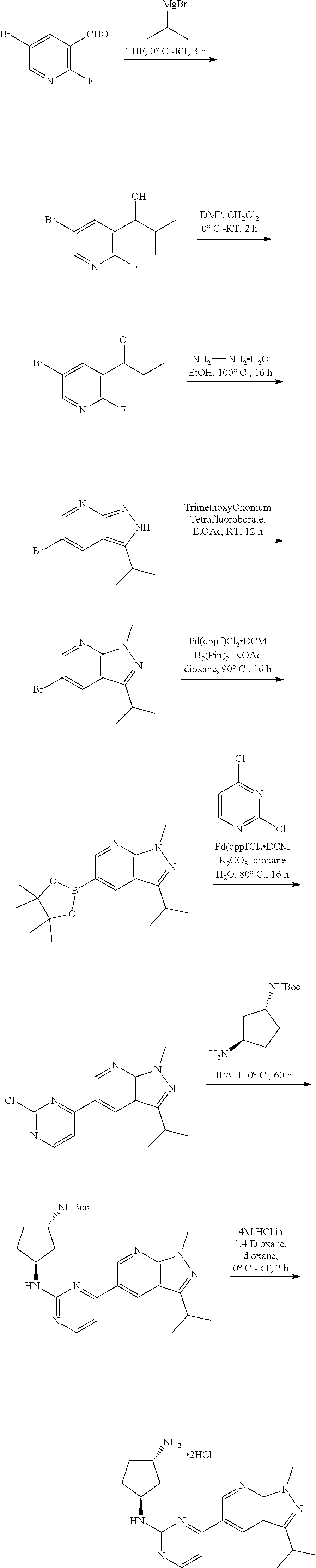

### Synthesis of 1-(5-bromo-2-fluoropyridin-3-yl)-2-methylpropan-1-ol

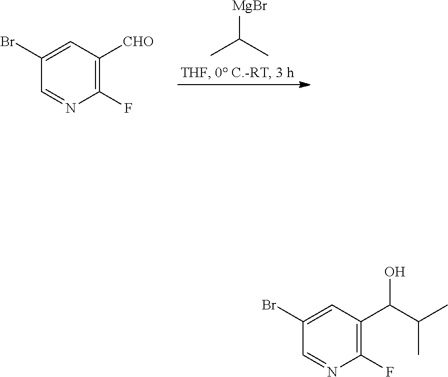

To a stirred solution of 5-bromo-2-fluoronicotinaldehyde (8 g, 39.2 mmol, 1.0 eq) in dry THF (80 mL) at 0° C., was added 1.5 M Isopropyl magnesium bromide in THF (31.3 mL, 47.1 mmol, 1.2 eq) and stirred at room temperature for 3 h. After completion of reaction by TLC, reaction mixture was diluted with sat NH₄Cl (50 mL) and extracted with EtOAc (2×100 mL). Organic layer was dried over sodium sulfate, concentrated, and purified by silica gel column (60-120 mesh, eluent: 12% EtOAc/petroleum ether) to afford 1-(5-bromo-2-fluoropyridin-3-yl)-2-methylpropan-1-ol (2.71 g, Yield: 28%) as yellow liquid. TLC system: EtOAc/petroleum ether (20:80), R_{f} value: ˜0.4. ¹H NMR (400 MHz, CDCl₃) δ 8.16 (dd, J=2.4 & 1.2 Hz, 1H), 8.04-8.01 (m, 1H), 4.72 (t, J=4.8 Hz, 1H), 1.99-1.97 (m, 2H), 0.93 (d, J=6.8 Hz, 3H), 0.87 (d, J=6.8 Hz, 3H).

### Synthesis of 1-(5-bromo-2-fluoropyridin-3-yl)-2-methylpropan-1-one

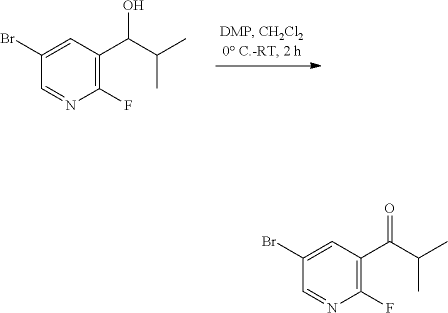

To a stirred solution of 1-(5-bromo-2-fluoropyridin-3-yl)-2-methylpropan-1-ol (3) (2.7 g, 10.9 mmol, 1.0 eq) in DCM (54 mL) at 0° C. was added DMP (6.95 g, 16.4 mmol, 1.5 eq) and stirred at room temperature for 2 h. After completion of reaction by TLC, diluted with sat NaHCO₃ solution (30 mL) and extracted with DCM (2×50 mL). The organic layer was separated, dried over sodium sulfate and concentrated to provided 3 g crude which was purified by silica gel column (60-120 mesh, eluent: 4% EtOAc/petroleum ether) to afford 1-(5-bromo-2-fluoropyridin-3-yl)-2-methylpropan-1-one (2.3 g, yield: 86%) as yellow liquid. TLC system: EtOAc/petroleum ether (20:80), R_{f} value: ˜0.85.

¹H NMR (400 MHz, CDCl₃) δ 8.41 (dd, J=2.4 &1.2 Hz, 1H), 8.35 (dd, J=8.0 &2.4 Hz, 1H), 3.44-3.40 (m, 1H), 1.20 (d, J=6.8 Hz, 6H).

### Synthesis of 5-bromo-3-isopropyl-2H-pyrazolo[3,4-b]pyridine

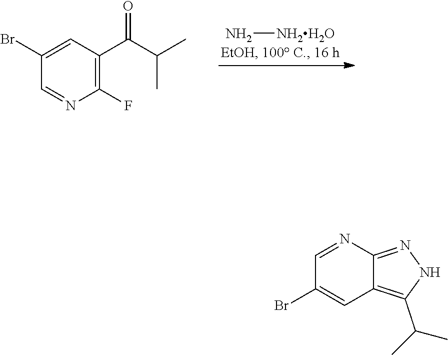

To a stirred solution of 1-(5-bromo-2-fluoropyridin-3-yl)-2-methylpropan-1-one (2.3 g, 9.38 mmol, 1.0 eq) in EtOH (23 mL, 10 Vol) was added 85% Hydrazine hydrate (12 mL, 5 Vol) and stirred at 100° C. for 16 h. After completion of reaction by TLC, the reaction mixture was evaporated. diluted with water (80 mL) and extracted with ethyl acetate (3×50 mL). Organic layer was separated, dried over sodium sulfate and concentrated to provided 2.6 g crude which was purified by silica gel column (60-120 mesh, eluent: 9% to 12% EtOAc/petroleum ether) to afford 5-bromo-3-isopropyl-2H-pyrazolo[3,4-b]pyridine (1.1 g, yield: 49%) as off-white solid. TLC system: EtOAc/petroleum ether (20:80), R_{f} value: ˜0.30. LC/MS Retention time=3.25 min, 240.0 [M+H]⁺.

¹H NMR (400 MHz, CDCl₃) δ 11.05 (brs, 1H), 8.59 (d, J=2.0 Hz, 1H), 8.27 (d, J=2.0 Hz, 1H), 3.41-3.36 (m, 1H), 1.48 (d, J=6.8 Hz, 6H).

### Synthesis of 5-bromo-3-isopropyl-1-methyl-1H-pyrazolo[3,4-b]pyridine

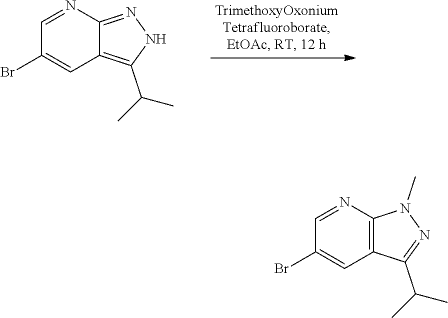

To a stirred solution of 5-bromo-3-isopropyl-2H-pyrazolo[3,4-b]pyridine (1.0 g, 4.18 mmol, 1.0 eq) in EtOAc (40 mL, 40 Vol) was added Trimethyloxonium tetrafluoroborate (3.71 g, 16.4 mmol, 4 eq) and stirred at room temperature for 12 h. After completion of reaction by TLC, diluted with cold water (100 mL) and extracted with EtOAc (2×80 mL). The organic layer was separated, dried over sodium sulfate and concentrated to provided 1.5 g crude which was purified by silica gel column (60-120 mesh, eluent: 8% to 11% EtOAc/petroleum ether) to afford 5-bromo-3-isopropyl-1-methyl-1H-pyrazolo[3,4-b]pyridine (280 mg, Yield: 27%) as yellow liquid. TLC system: EtOAc/petroleum ether (20:80), R_{f} value: ˜0.7;

¹H NMR (400 MHz, CDCl₃) δ 8.51 (d, J=2.0 Hz, 1H), 8.18 (d, J=2.0 Hz, 1H), 4.06 (s, 3H), 3.36-3.29 (m, 1H), 1.43 (d, J=7.2 Hz, 6H).

### Synthesis of 3-isopropyl-1-methyl-5-(4,4,5,5-tetramethyl-1,3,2-dioxaborolan-2-yl)-1H-pyrazolo[3,4-b]pyridine

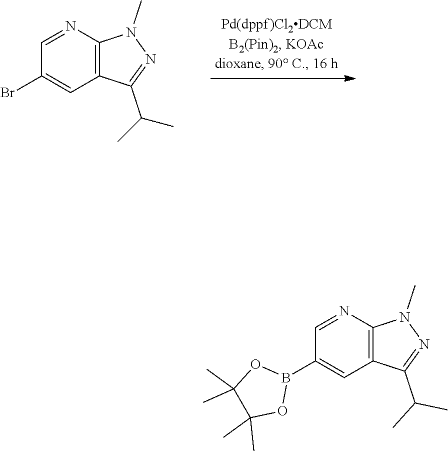 [SMILES: CC(C)c1nn(C)c2ncc(Br)cc12 ; CC(C)c1nn(C)c2ncc(B3OC(C)(C)C(C)(C)O3)cc12]

To a stirred solution of 5-bromo-3-isopropyl-1-methyl-1H-pyrazolo[3,4-b]pyridine (280 mg, 1.11 mmol, 1.0 eq) in 1,4-dioxane (6 mL) was added bis(pinacolato)diborane (337 mg, 1.32 mmol, 1.2 eq), potassium acetate (271 mg, 2.77 mmol, 2.5 eq) and degassed for 5 min. Later added Pd(dppf)Cl₂·DCM (45 mg, 0.055 mmol, 0.05 eq) and stirred at 90° C. for 16 h. After completion of reaction by TLC. Volatiles removed under vacuum, diluted with water (50 mL) and extracted with ethyl acetate (3×20 mL). The organic layer was separated, washed with brine solution (10 mL), dried over sodium sulfate and concentrated to provide 3-isopropyl-1-methyl-5-(4,4,5,5-tetramethyl-1,3,2-dioxaborolan-2-yl)-1H-pyrazolo[3,4-b]pyridine (360 mg, crude) as black semi solid. TLC system: EtOAc/petroleum ether (20:80), R_{f} value: ˜0.45. Cr: LC/MS: 56% boronic acid m/z, 220.1 [M+H]⁺.

### Synthesis of 5-(2-chloropyrimidin-4-yl)-3-isopropyl-1-methyl-1H-pyrazolo[3,4-b]pyridine

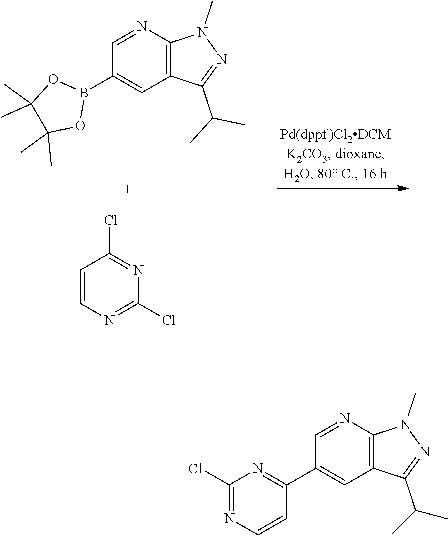 [SMILES: CC(C)c1nn(C)c2ncc(B3OC(C)(C)C(C)(C)O3)cc12 ; Clc1ccnc(Cl)n1 ; CC(C)c1nn(C)c2ncc(-c3ccnc(Cl)n3)cc12]

To a stirred solution of 3-isopropyl-1-methyl-5-(4,4,5,5-tetramethyl-1,3,2-dioxaborolan-2-yl)-1H-pyrazolo[3,4-b]pyridine (360 mg, 1.19 mmol, 1 eq) in 1,4-dioxane and water (3:1) (7 mL) was added 4-chloro-2-(methylthio)pyrimidine (213 mg, 1.43 mmol, 1.2 eq), K₂CO₃ (412 mg, 2.99 mmol, 2.5 eq) and degassed for 5 min. Later added Pd(dppf)Cl₂DCM (48 mg, 0.06 mmol, 0.05 eq) and stirred at 80° C. for 16 h. After completion of reaction by TLC, RM was evaporated, diluted with water (30 mL) and extracted with ethyl acetate (2×40 mL). Organic layer was separated, dried over sodium sulfate and concentrated to provided 2.6 g crude which was purified by silica gel column (100-200 mesh, eluent: 18-22% EtOAc/petroleum ether) to afford 5-(2-chloropyrimidin-4-yl)-3-isopropyl-1-methyl-1H-pyrazolo[3,4-b]pyridine (230 mg, semi-pure) as light yellow liquid. TLC system: EtOAc/petroleum ether (30:70), R_{f} value: ˜0.2. LC/MS: Retention time=3.56 min, 288.1 [M+H]⁺; 68% purity.

### Synthesis of tert-butyl ((1S,3S)-3-((4-(3-isopropyl-1-methyl-1H-pyrazolo[3,4-b]pyridin-5-yl)pyrimidin-2-yl)amino)cyclopentyl)carbamate

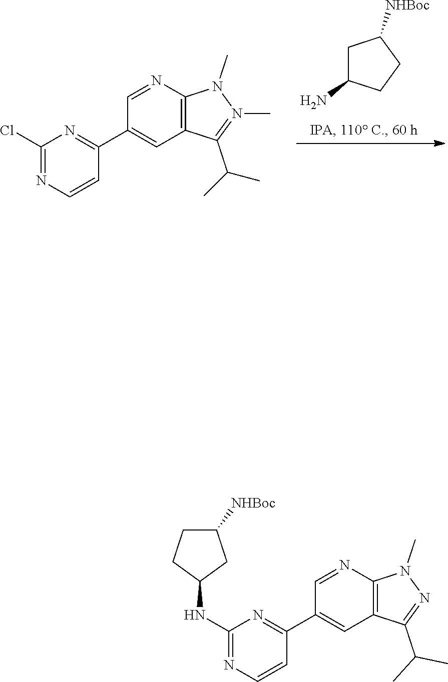

To a stirred solution of 5-(2-chloropyrimidin-4-yl)-3-isopropyl-1-methyl-1H-pyrazolo[3,4-b]pyridine (230 mg, 0.80 mmol, 1 eq) in IPA (4 mL) was added tert-butyl ((1S,3S)-3-aminocyclopentyl)carbamate (240 mg, 1.20 mmol, 1.5 eq) and stirred at 110° C. for 60 h. TLC showed new polar spot and absence of starting material. Volatiles removed under vacuum, diluted with water (20 mL) and extracted with EtOAc (2×20 ml). Organic layer was dried over Na₂SO₄, filtered, concentrated and purified by reverse phase column (eluent: 32-59% acetonitrile and 0.01% formic acid in water) to afford tert-butyl ((1S,3S)-3-((4-(3-isopropyl-1-methyl-1H-pyrazolo[3,4-b]pyridin-5-yl)pyrimidin-2-yl)amino)cyclo pentyl)carbamate (120 mg, 33%) as brown solid. TLC system: EtOAc/petroleum ether (50:50), R_{f} value: ˜0.40; LC/MS Retention time=3.65 min, 452.2 [M+H]⁺.

¹H NMR (400 MHz, CDCl₃): δ 9.17 (brs, 1H), 8.70 (s, 1H), 8.34 (d, J=4.8 Hz, 1H), 7.02 (d, J=5.2 Hz, 1H), 5.32 (bs, 1H), 4.57 (brs, 1H), 4.52-4.47 (m, 1H), 4.15-4.12 (m, 4H), 3.49-3.40 (m, 1H), 2.34-2.23 (m, 2H), 2.05-1.95 (m, 2H), 1.50-1.45 (m, 17H).

### Synthesis of (1S,3S)—N1-(4-(3-isopropyl-1-methyl-1H-pyrazolo[3,4-b]pyridin-5-yl)pyrimidin-2-yl)cyclopentane-1,3-diamine HCl salt

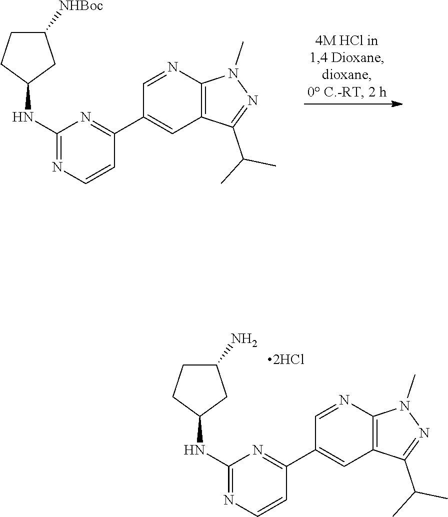 [SMILES: CC(C)c1nn(C)c2ncc(-c3ccnc(N[C@H]4CC[C@H](NC(=O)OC(C)(C)C)C4)n3)cc12 ; CC(C)c1nn(C)c2ncc(-c3ccnc(N[C@H]4CC[C@H](N)C4)n3)cc12]

To a stirred solution of tert-butyl ((1S,3S)-3-((4-(3-isopropyl-1-methyl-1H-pyrazolo[3,4-b]pyridin-5-yl)pyrimidin-2-yl)amino)cyclopentyl)carbamate (120 mg, 0.27 mmol, 1 eq.) in 1,4-Dioxane (1.2 mL) at 0° C., was added 4M HCl in 1,4-Dioxane (0.6 mL) and stirred for 2 h at room temperature. After completion of starting material, volatiles removed under vacuum, triturated with diethyl ether (2×3 mL) and lyophilized. Obtained material was purified by reverse phase column (eluted with 32% acetonitrile and 0.01% formic acid in water) to afford (1S,3S)—N1-(4-(3-isopropyl-1-methyl-1H-pyrazolo[3,4-b]pyridin-5-yl)pyrimidin-2-yl)cyclopentane-1,3-diamine HCl salt (40 mg, 43%) as brown gummy solid. TLC system: MeOH/DCM (10:90), R_{f} value: ˜0.1; LC/MS Retention time=2.27 min, 352.4 [M+H]⁺.

¹H NMR (400 MHz, CD₃OD) δ 9.47 (brs, 1H), 9.15 (s, 1H), 8.36 (d, J=6.4 Hz, 1H), 7.77 (d, J=6.4 Hz, 1H), 4.55-4.49 (br, 1H), 4.10 (s, 3H), 3.92-3.88 (m, 1H), 3.54-3.50 (m, 1H), 2.47-2.38 (m, 2H), 2.31-2.28 (m, 2H), 1.94-1.80 (m, 2H), 1.48 (d, J=6.8 Hz, 6H).

### Example 13

### Synthesis of Compound I-13

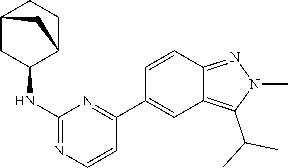 [SMILES: CC(C)c1c2cc(-c3ccnc(N[C@H]4C[C@@H]5CC[C@H]4C5)n3)ccc2nn1C]

### Synthesis of N-((1R,2S,4R)-bicyclo[2.2.1]heptan-2-yl)-4-(3-isopropyl-2-methyl-2H-indazol-5-yl)pyrimidin-2-amine

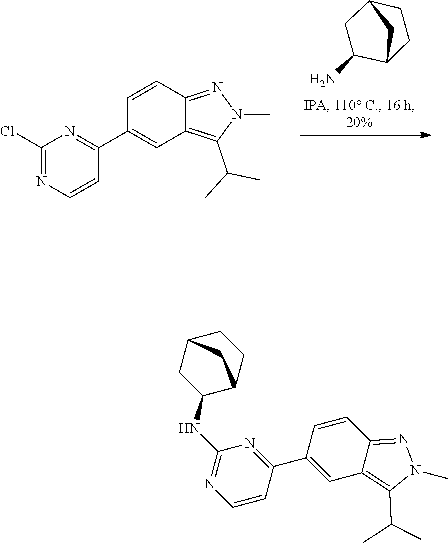

In a sealed tube, a solution of 5-(2-chloropyrimidin-4-yl)-3-isopropyl-2-methyl-2H-indazole (300 mg, 1.04 mmol, 1 eq) and (1S,2S,4R)-bicyclo[2.2.1]heptan-2-amine (250 mg, 1.36 mmol, 1.3 eq) in isopropyl alcohol (3 mL) was stirred at 110° C. for 16 h. After completion of reaction by TLC, the reaction mixture was evaporated and purified by reverse phase column [eluted in 56% of acetonitrile in 0.1% formic acid in water] to afford N-((1R,2S,4R)-bicyclo[2.2.1]heptan-2-yl)-4-(3-isopropyl-2-methyl-2H-indazol-5-yl)pyrimidin-2-amine as off-white solid (77 mg, yield: 20%). TLC system: EtOAc/hexane (1:1), R_{f} value: ˜0.2; LC/MS (m/z): 362.3 (M+H)⁺

¹H NMR (400 MHz, CDCl₃) δ: 8.60 (brs, 1H), 8.30 (d, J=4.8 Hz, 1H), 7.88 (d, J=9.2 Hz, 1H), 7.67 (d, J=9.2 Hz, 1H), 6.99 (d, J=5.2 Hz, 1H), 5.08-5.06 (m, 1H), 4.16 (s, 3H), 3.89 (brs, 1H), 3.53-3.46 (m, 1H), 2.43 (brs, 1H), 2.33 (brs, 1H), 1.95-1.89 (m, 1H), 1.60-1.57 (m, 6H), 1.54-1.49 (m, 2H), 1.38-1.33 (m, 2H), 1.32-1.28 (m, 2H).

### Example 14

### Synthesis of Compound I-14

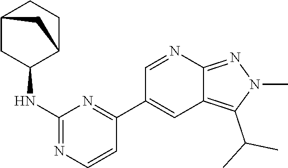 [SMILES: CC(C)c1c2cc(-c3ccnc(N[C@H]4C[C@@H]5CC[C@H]4C5)n3)cnc2nn1C]

### Synthesis of N-((1R,2S,4R)-bicyclo[2.2.1]heptan-2-yl)-4-(3-isopropyl-2-methyl-2H-pyrazolo[3,4-b]pyridin-5-yl)pyrimidin-2-amine TFA salt (BLX-3051)

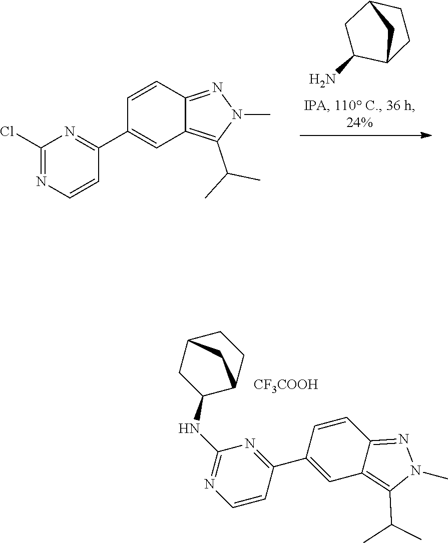

In a sealed tube, a solution of 5-(2-chloropyrimidin-4-yl)-3-isopropyl-2-methyl-2H-pyrazolo[3,4-b]pyridine (300 mg, 1.04 mmol, 1 eq) and (1S,2S,4R)-bicyclo[2.2.1]heptan-2-amine (150 mg, 1.35 mmol, 1.3 eq) in IPA (5 mL) was stirred at 110° C. for 36 h. After completion of reaction by TLC, reaction mixture was evaporated and purified by reverse phase column [eluted in 45% of acetonitrile in 0.1% formic acid in water] to afford N-((1R,2S,4R)-bicyclo[2.2.1]heptan-2-yl)-4-(3-isopropyl-2-methyl-2H-pyrazolo[3,4-b]pyridin-5-yl)pyrimidin-2-amine TFA salt as pale yellow solid (89 mg, yield: 24%). TLC system: EtOAc/hexane (60:40), R_{f} value: ˜0.2; LC/MS (m/z): 363.3 (M+H)⁺

¹H NMR (400 MHz, DMSO-d₆) δ: 9.32 (s, 1H), 9.10 (brs, 1H), 8.39 (d, J=5.6 Hz, 1H), 7.72 (brs, 1H), 7.48 (d, J=4.0 Hz, 1H), 4.17 (s, 3H), 3.79 (brs, 1H), 3.64-3.57 (m, 1H), 2.48-2.45 (m, 1H), 2.33-2.27 (m, 2H), 1.76-1.71 (m, 1H), 1.56-1.48 (m, 10H), 1.29-1.26 (m, 1H), 1.16-1.12 (m, 2H).

### Biological Example 1

### Cytotoxicity Assay

Multiplexing the LDH-Glo Cytotoxicity Assay and CellTiter-Glo 2D Cell Viability Assay. The RWPE-1 Normal Prostate Cells were treated with compound I-2 and AZD-4573 in a 10-dose IC₅₀ mode in duplicate, with 3-fold serial dilution starting at 100 μM with 5000 cells/well in 96-well format for 72 hours. Change in toxicity was measured using the LDH-Glo Cytotoxicity Assay by combining 50 μL diluted samples with 50 μL LDH Detection Reagent and recording luminescence after 1 hour incubation. After samples were removed for LDH measurement, an equal volume of CellTiter Glo 2D Reagent was added to the remaining cell suspension and luminescence was recorded after 30-minute incubation period.

The cytoxicity of compound I-2 was tested in RWPE-1 normal healthy donor prostate cells using a very sensitive assay measuring the release of lactate dehydrogenase (LDH), a cytosolic enzyme that is released into the cell culture media when plasma membrane integrity is lost. Data with both the LDH-Glo assay and cell viability assay using CellTiter-Glo show that cytotoxicity of normal cells does not occur until compound I-2 reaches concentrations >15-fold higher than that needed to kill most prostate cancer cells (IC₅₀ values of 5.26 μM in these normal cells). Reference compound AZD-4573 is highly toxic to normal prostate cells.

TABLE 2
Cytotoxicity results for compound I-2 vs AZD-4573

IC₅₀ (μM)

| Assay | CellTiter-Glo | LDH-Glo |
|---|---|---|
| Compound I-2 | 5.26 | 6.7 |
| AZD-4573 | 0.0084 | 0.151 |

### Biological Example 2

### CellTiter-Glo Assay

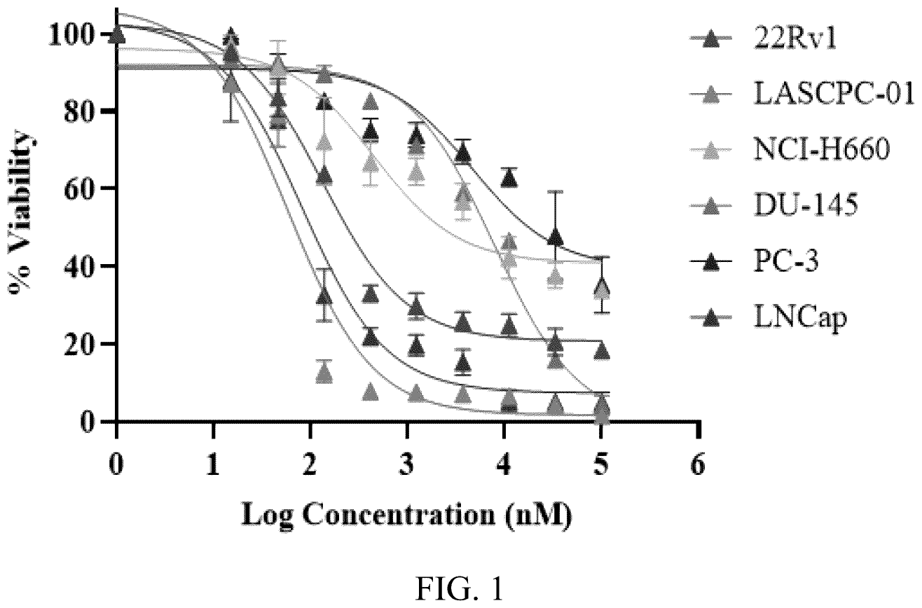

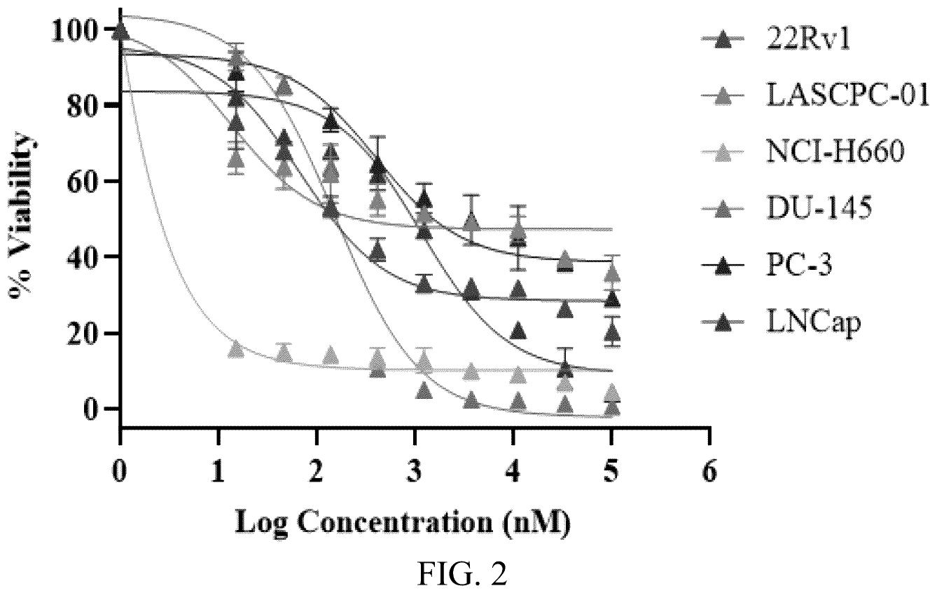

CellTiter-Glo® 2.0 Luminescent cell viability assay reagent was purchased from Promega and 22Rv1 and LASCPC-01 cell lines were purchased from ATCC. The test compound, Compound I-2 was dosed in 10-dose IC₅₀ mode in duplicate with 3-fold serial dilution starting at 100 μM with 4000 cells/well (adherent) in 96-well format and treatment time of 72 hours was used for all the chosen cell lines. An equal volume of CellTiter-Glo 2D Reagent was added to the cell suspensions and luminescence was recorded after 30-minute incubation period. The results are shown in FIGS. 1 and 2. FIG. 1 illustrates cellular antiproliferative efficacy of compound I-2 on six prostate cancer cell lines over various concentrations. FIG. 2 illustrates cellular antiproliferative efficacy of a benchmark CDK9 inhibitor AD-4573 on six prostate cancer cell lines over various concentrations. The experimental data FIGS. 1 and 2 are summarized in Table 3.

TABLE 3
Cell Titer-Glo Assay results

2D CellTiter-Glo IC₅₀ (nM)

| Cell lines | Compound I-2 | AZD-4573 |
|---|---|---|
| 22Rv1 | 132 | 67.14 |
| LASCPC-01 | 58.02 | 13.51 |
| NCI-H660 | 447.7 | 0.2211 |
| DU-145 | 7718 | 150.7 |
| PC-3 | 45.94 | 400 |
| LNCap | 80.44 | 1152 |

### Biological Example 3

### Colony Formation Assay

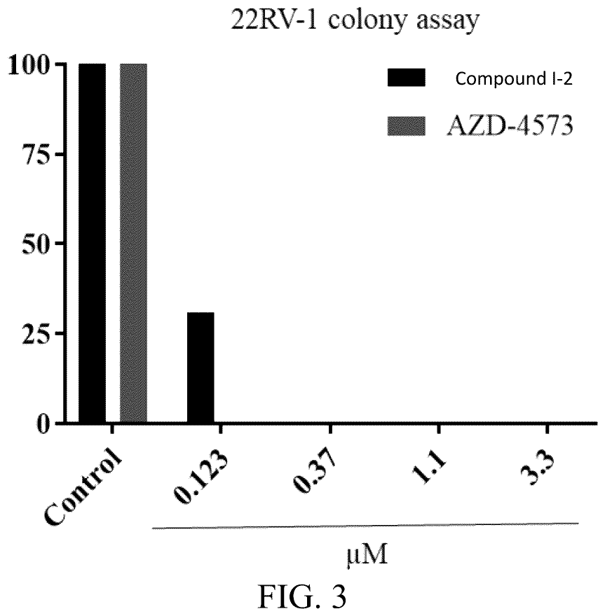

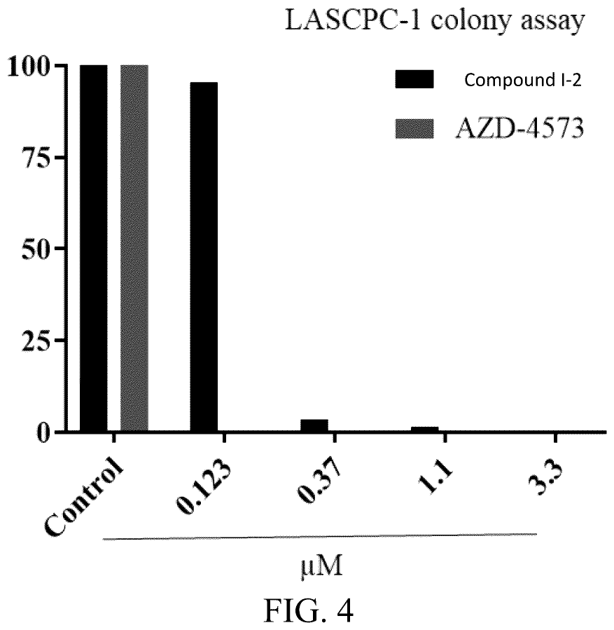

Colony formation assay was performed on 2 Different NEPC Cell Lines. Cells were seeded at 800 cells per well in a 6-well plate and left to adhere for 24 hours in complete growth media. Treatment with compound I-2 ranged from NT to 10.0 μM for 14 days. Cells were fixed with a 3:1 methanol/acetic acid mixture and stained with methylene blue. SEM from two independent experiments. (*P<0.05, **P<0.01, ***P<0.005 vs. DMSO control). The results showed that compound I-2 more significantly inhibited the proliferation and colony formation of 22Rv1 than that of LASCPC-01, which is illustrated in FIGS. 3 and 4.

### Biological Example 4

### Annexin V/7-AAD Apoptosis Assay

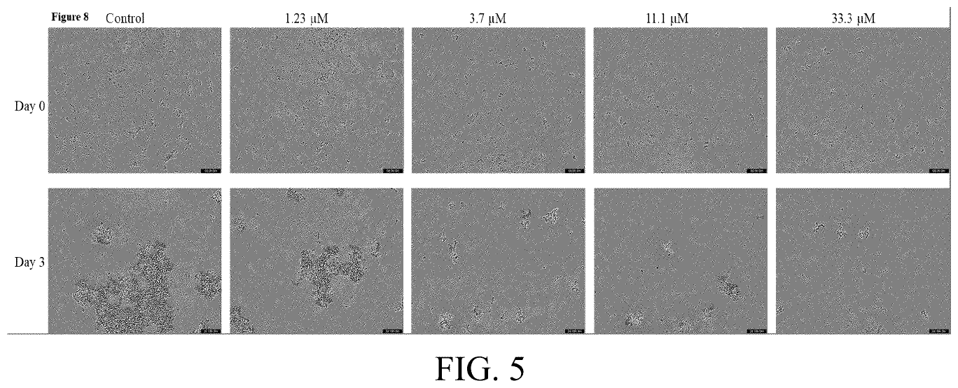

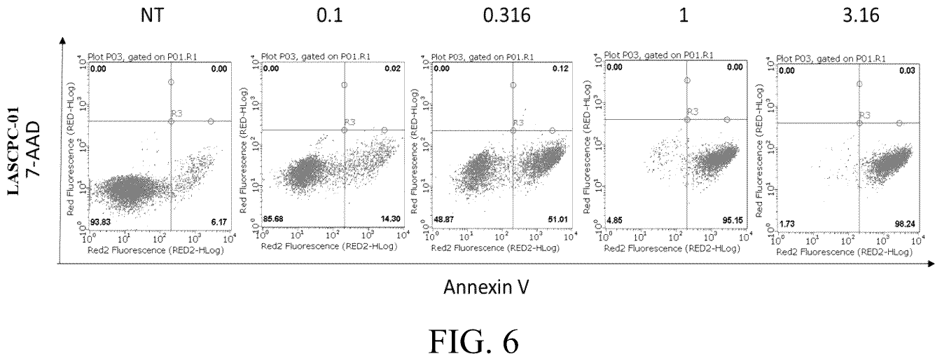

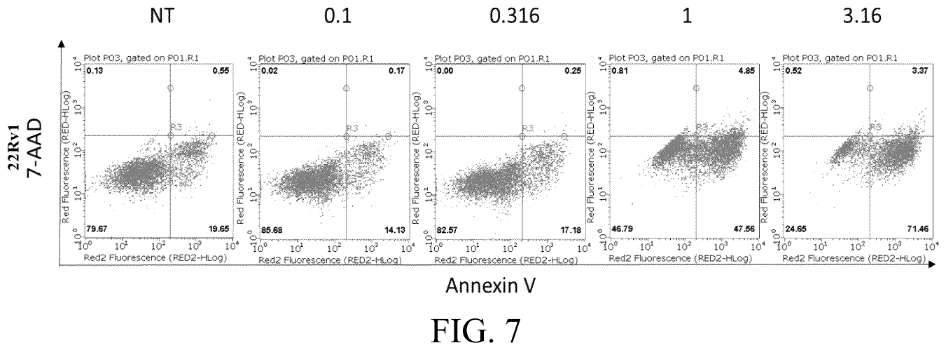

Apoptosis of 2 NEPC cells by Annexin V/7-AAD Staining: Apoptosis induced by CDK9 inhibitor compound I-2 was assessed by Annexin V/7-AAD double staining and FACS analysis using a GUAVA Flow Cytometer. Compound I-2 treatments increased apoptosis in a dose dependent manner Real-time measurements of apoptosis induced by compound I-2 treatments in prostate cancer cells were determined using IncuCyte S3 Live-Cell Analysis System. The representative images and bar graph from LASCPC-01 cells are shown in FIG. 5. Additionally, apoptosis induced by CDK9 inhibitor compound I-2 was also assessed by Annexin V/7-AAD double staining and FACS analysis. Compound I-2 treatments increased apoptosis in a dose dependent manner and the Annexin V/7-AAD staining results are as shown in FIGS. 6 and 7.

Treatment with compound I-2 increased the apoptosis in a dose dependent manner and is comparable to the results obtained by CellTiter-Glo assay described in herein. NEPC cells were treated for 72 hours and showed LASCPC-01, 22Rv1 and C₄-2 cells were very sensitive to treatment with compound I-2. The treatment resulted in 69, 71 and 84% cell death, respectively, which is comparable to 2D CellTiter-Glo assay results.

### Biological Example 5

### Western Blot

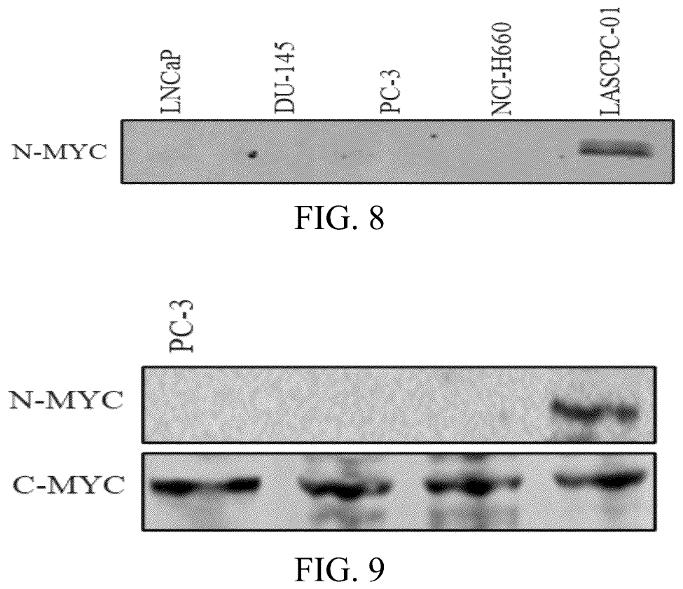

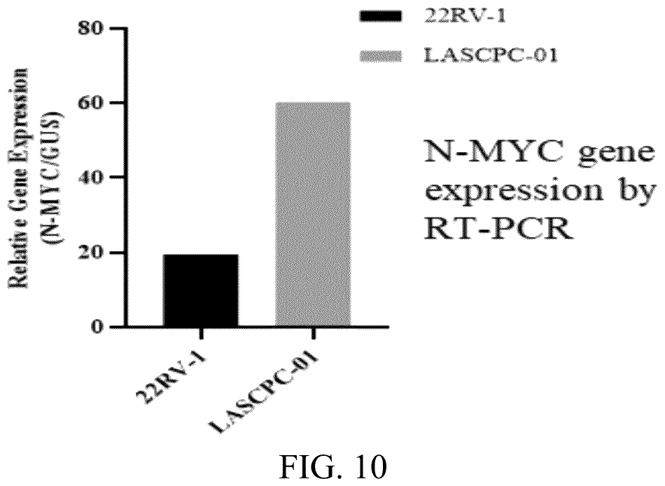

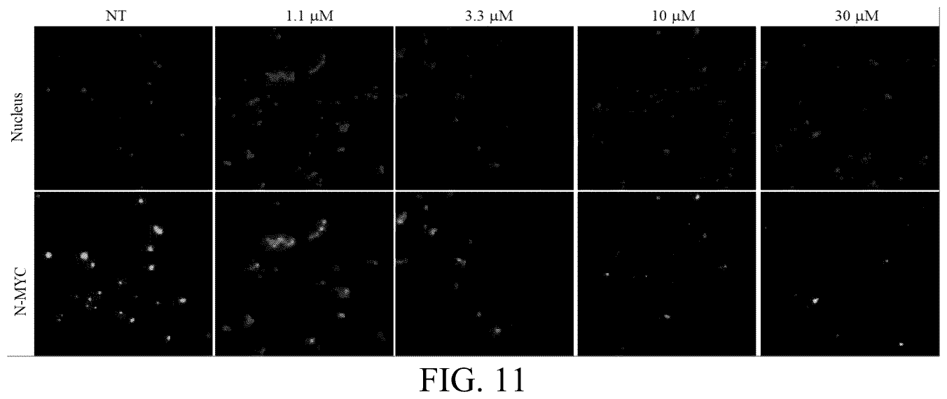

Two NEPC cell lines (22Rv1 & LASCPC-01) were treated with compound I-2 for 6 and 24 hours. Cytoplasmic and nuclear extracts from these cells were separated using NE-PER Nuclear and Cytoplasmic Extraction Kit (ThermoFisher). Cell extracts were separated on 4-12% Bis-Tris NuPAGE gel and then transferred onto 0.45 Nitrocellulose Membrane, followed by 30 min blocking with Pierce Fast Blocking Buffer, and then incubated overnight at 4° C. with indicated antibodies. Relative protein expression of CDK9, N-Myc and other downstream signaling was quantified by GraphPad Prism 8 and ImageJ Software RT-PCR Gene. The results are shown in FIGS. 8-10.

### Biological Example 6

### Expression

Total RNA was extracted using RNeasy Mini Kit (Qiagen). cDNA was synthesized using iScript cDNA Synthesis Kit (Bio-Rad, Hercules, CA). Quantitative RT-PCR was performed with SsoAdvanced SYBR Green Supermix (Bio-Rad) on a CFX96 Real-Time PCR Detection System (Bio-Rad). Data was normalized to GUS and percentage of gene expression was determined by ΔCt method.

### Biological Example 7

### ADP-Glo Kinase Assay

ADP-Glo assay kit was purchased from Promega and the CDK9/CycT1 protein and PDKtide substrate were purchased from SignalChem. Compounds were dosed in 10-dose IC₅₀ mode in duplicate with 3-fold serial dilution starting at 100 PM. The reaction was started by pre-incubating 4 μL of protein (20 nM), 2 μL of substrate (100 μM), and 2 μL of serial diluted drug in 1× assay buffer (50 mM HEPES, 3 mM MgCl₂, 3 mM MnCl₂, and 1 mM DTT) for 30 minutes. After pre-incubating, 4 μL of ATP (1 μM) was added to each well and incubated for an additional 90 minutes. The ADP-Glo Reagent (10 μL) and Kinase Detection Reagent (20 μL) were added to each well and incubated as recommended by Promega. The reaction was quantified by measuring luminescence and the IC₅₀ was calculated using GraphPad Prism software.

### Biological Example 8

### Immunofluorescence Staining

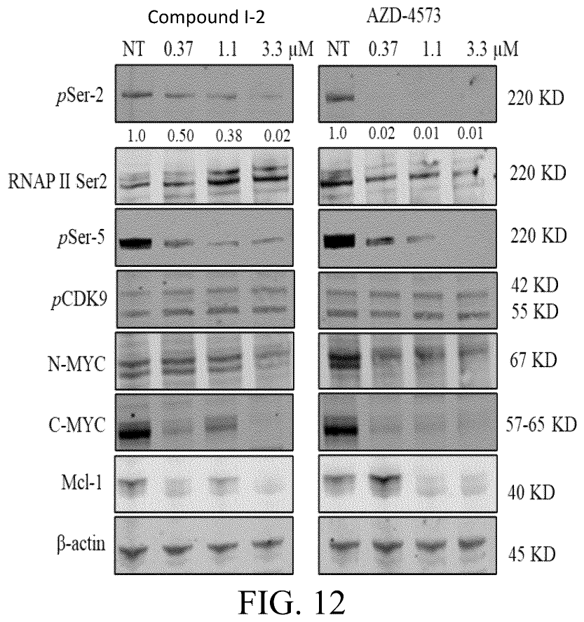

LASCPC-01 NEPC cells were treated with compound I-2 starting from NT, 1.1, 3.3 10, and 30 μM for 6 hours. After 6 hours, the cells were fixed with 4% formaldehyde and probed with primary N-Myc antibody overnight. The cells were then incubated with secondary antibody conjugated with Alexa Fluor 488 and counterstained with Hoechst 33342. Images were taken with Nikon Automated Widefield CCD Camera and were quantified using ImageJ and GraphPad Prism 8 software, which is shown in FIG. 11

### Biological Example 9

### Cell Cycle Arrest Assay

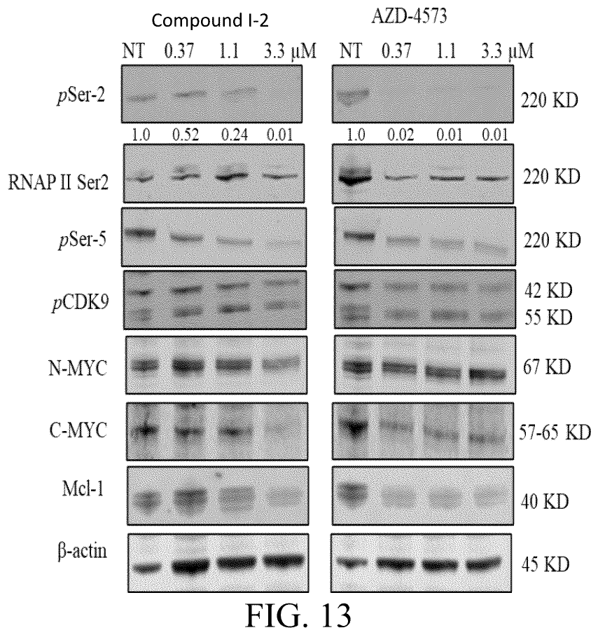

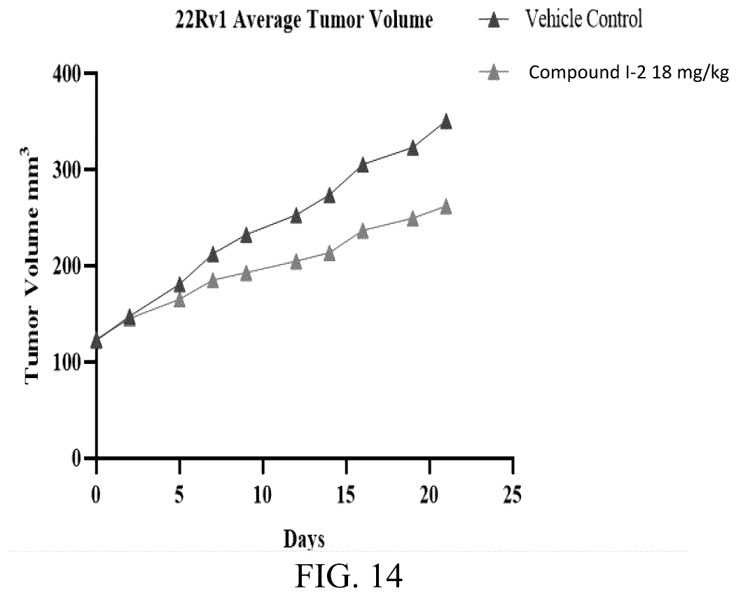

NEPC cells were treated with compound I-2 starting from NT, 1, and 3.16 μM for 8 hours. Then cells were harvested with PBS and fixed with ice-cold 70% ethanol at −20° C. overnight. The cells were then washed with ice-cold PBS 2× and then re-suspended in DAPI staining buffer and incubated in the dark for 30 minutes. The cells were analyzed using BD Fortessa flow cytometer and cell cycle distribution was analyzed using FlowJo and GraphPad Prism 8 Software. Western blot analysis of nuclear extract showed that compound I-2 inhibited the CDK9 downstream targets. Representative blots from 22Rv1 and LASCPC-01 are shown in FIGS. 12-13.

### Biological Example 10

### Cell Cycle Arrest Assay

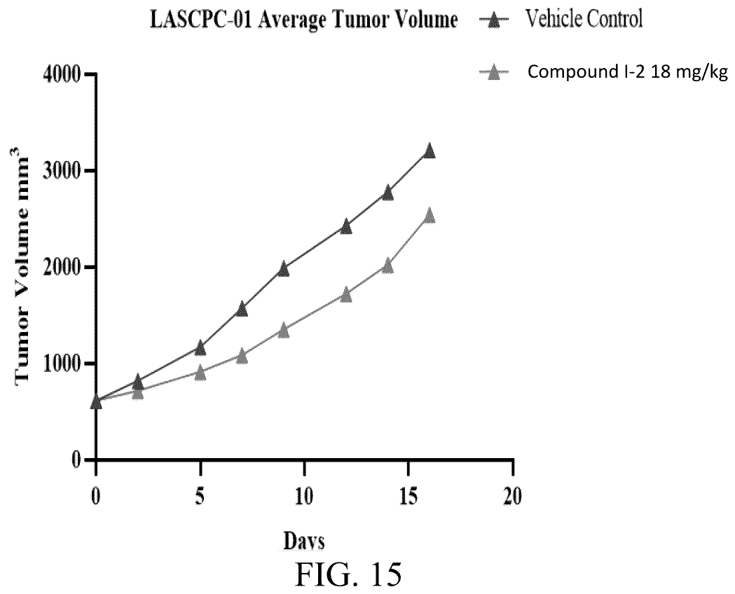

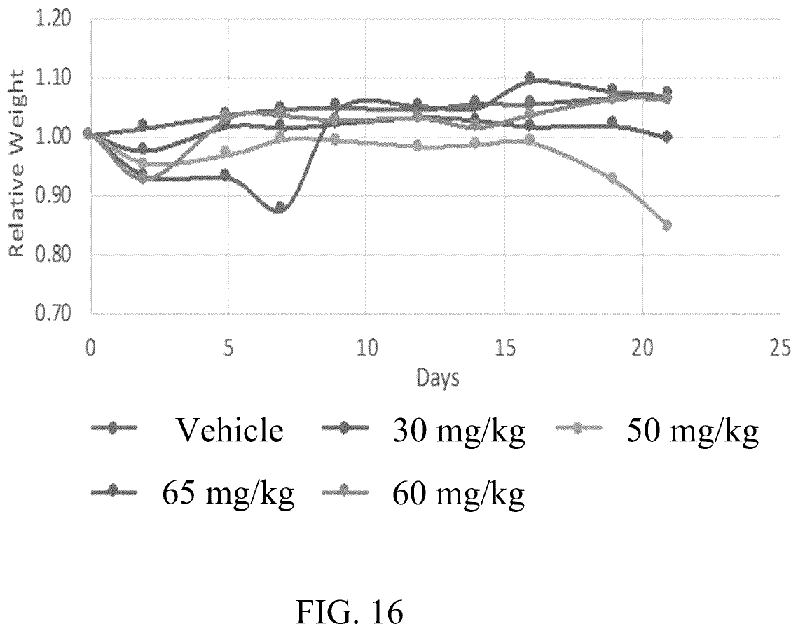

Pilot in vivo xenograft 22Rv1 and LASCPC-01 engraftment studies are described herein. Animals: Male BALB/c nude mice (8 week age). NEPC Cell lines: 22Rv1 & LASCPC-01. Number of cells: 22Rv1 (1.2×10⁶) & LASCPC-01 (10⁶). Sub. Q. Tumor volume was measured two times per week by calipers and calculated by length×(width²)/2. When tumors reached approximately 100-500 mm³, mice were randomized and selected for treatment with compound I-2 as a TFA salt with 18-mg/kg oral dose. This experimental data is illustrated in FIGS. 14-15.

### Biological Example 11

### Kinase Profiling

CDKs family kinases profiling assays were performed with DiscoveRx Matrix of Kds KINOMEscan (KdELECT) and Reaction Biology using miniaturized radioisotope-based assay platform (³³P). IC₅₀ value estimated based on the best curve fitting available. Compound I-2 & AZD4573 (CAS #2057509-72-3) exhibited IC₅₀ values of 4.2 and <5.0 nM respectively against CDK9.

In summary, Compound I-2 exhibited promising selectivity with 3 to 50-fold against closest CDK homologs tested.

TABLE 4
Compound I-2 10-dose IC₅₀ values (nM/Kds)
against homologs of CDK9.

| Kinase | CDK9 |
|---|---|
| CDK2/cyclinA | 17.60 IC₅₀/30 Kd |
| CDK3/cyclinE | 115 IC₅₀/31 Kd |
| CDK4/cyclinD | 22.80 IC₅₀/22 Kd |
| CDK5/P21 | 245 |
| CDK7/cyclinH | 119 |
| CDK9/cyclinT | <5/4.2 |
| Staurosporine | <1 |

### Biological Example 12

### Off-Target Inhibition Test

Compound I-2 has potent activity in cell-free assays with an IC₅₀ of 4.2 nM, in cell assays with an IC₅₀ of 15.58 nM in NanoBRET target engagement assay, and IC₅₀ values of 132, 58, 28 and 31 nM in inhibiting 22Rv1, LASCPC-01, C4-2 and C4-2B cells of NEPC origin. Compound I-2 reduced the pCDK9, pSer2 and its downstream N-MYC, C-MYC and MCL-1 target in newly diagnosed and resistant prostate cancer patient samples, indicating its response to overcoming resistance and anti-tumor efficacy. Data suggests that compound I-2 has strong potential for development as a candidate for NEPC therapy.

Off-target inhibition of other kinases was analyzed using the scanMAX panel. scanMAX comprises 468 kinases including lipid and atypical kinases. Compound I-2 was tested at a concentration of 1 μM where the target of interest, CDK9 had 2.7% activity comparable to its IC₅₀ of 4.2 nM. At the 1 μM concentration, activity was found against 9 other kinases (CLK/2.9 Kd)), DYRKA1 (9 Kd), ERK8 (5.5 Kd), FLT3/ITD(D835V) (1.2 Kd), Haspin (2.8 Kd), PCTK1 (8.2 Kd), PRKD3 (230 Kd), TAOK1 (19 Kd), TAOK3 (9.2 Kd) each of which exhibited 0-1% of control activity. These 9 off-target kinases were further tested for their Kds and IC₅₀ values and as shown in Table 3 above. From this selectivity profiler screen, the key off target kinase is FLT3. The FDA has approved 48 kinase inhibitors, of which 11 carry 2, 4-disubstituted pyrimidines like compound I-2 and demonstrated a similar off-target profile. The structure of compound I-2 includes a unique chemotype with high cell permeation, which supports superior overall selectivity and efficacy when compared to the other 11 approved kinase inhibitors.

### Biological Example 13

### Target Engagement Assay in HEK293 Cells

HEK293 cells were transfected with 1 μg CDK9 and 9 μg Cyclin T1 DNA and the transfected cells were treated with compound I-2 (starting at 100 μM, 10-dose with 3-fold dilution) and reference compound Dinaciclib (starting at 10 PM, 10-dose with 3-fold dilution) was used. Curve fits were performed only when % NanoBRET signal at the highest concentration of compounds was less than 55%. The fresh HEK293 cells procured from ATCC, and compounds treatment time was 1 hour. The results were plotted and IC₅₀ data was calculated as shown below in Table 4.

TABLE 5
NanoBRET Target Engagement Assay Results

| Compound | CDK9 + Cyclin T1 IC₅₀ value (nM) | |
|---|---|---|
| Compound I-2 | 15.58 | nM |
| Dinaciclib (Reference) | 5.05 | nM |

### Biological Example 14

### Cellular Antiproliferative Efficacy

CellTiter-Glo® 2.0 Luminescent cell viability assay reagent was purchased from Promega and 22Rv1, LASCPC-01, C4-2, C4-2B and NCI-H660, DU-145, PC-3 and LNCap cell lines were obtained from ATCC and a clinical core facility at Huntsman Cancer Institute. Compound I-2 and the reference compound AZD-4573 were tested in 10-dose IC₅₀ mode in duplicate with 3-fold serial dilution starting at 100 μM with 2500 cells/well (adherent) in 96-well format, and the treatment time of 72 hours was used for all the chosen cell lines. The results of cell viability were obtained and IC₅₀ values were calculated (see Table 5, below) showing sub-micromolar cell killing of these prostate cancer cells. Compound I-2 was particularly sensitive on all NEPC cells (see, e.g., results for 22Rv1, LASCPC-01, NCI-H660, C4-2, C4-2B).

TABLE 6
2D CellTiter-Glo Assay Results; IC₅₀ values are given in nM

| Cell Line | Compound I-2 | AZD4573 |
|---|---|---|
| 22Rv1 | 132 | 67.14 |
| LASCPC-01 | 58.02 | 13.51 |
| NCI-H660 | 447.7 | 0.2211 |
| C4-2 | 28.16 | 0.237 |
| C4-2B | 31.92 | 0.398 |
| DU-145 | 7718 | 150.7 |
| PC-3 | 4594 | 400 |
| LNCap | 80.44 | 1152 |

### Biological Example 15

### Colony Formation Assay 2

Prostate cancer cells were seeded at 400 cells per well in a 6-well plate and left to adhere for 24 hours in complete growth media, followed by treatment with compound I-2 at concentrations ranging from 0.123, 0.37, 1.1, 3.0 and 10 μM for 72 hours. Treatment resulted in complete inhibition of colony formation with all prostate cancer cells.

### Biological Example 16

### Fluorescence Polarization Assay

The Predictor™ hERG Fluorescence Polarization Assay Kit was used to perform hERG channel biochemical binding studies in the absence of radioligand. Assay performance was validated with a panel of established hERG channel blockers. Results from the Predictor™ assay demonstrate a high correlation with those obtained from patch clamp techniques. Compound I-2 was found to have no hERG activity and the measured IC₅₀ was 57.8 μM.

### Biological Example 17

### P450 Isozyme Inhibition Assay

P450 baculosome reagents (i.e., microsomes), were prepared from insect cells infected with recombinant baculovirus containing a human P450 isozyme and a rabbit NAPDH-P450 reductase since only a single P450 isozyme is expressed over human liver microsomes (HLM). Compound I-2 was incubated with baculosome plus substrate and the reaction was initiated by addition of NAPDH-450. At the end of incubation time, a stop solution was added to terminate the reaction that resulted in stable fluorescent signal compared with the compound I-2 and solvent control. The inhibition effect of compound I-2 was assessed against five P450 isozymes with results provided below.

TABLE 7
P450 Assay results

| P540 isozyme | IC₅₀ of compound I-2 (μM) |
|---|---|
| 1A2 | 29.4 |
| 2C19 | 28.3 |
| 2C9 | 50.8 |
| 2D6 | 100 |
| 3A4 | 31.1 |

### Biological Example 17

### Pharmacokinetic Study—Bioavailability

Intravenous administration of compound I-2 at 1 mg/kg for male Beagle Dogs, revealed moderate plasma clearance compared with normal hepatic blood flow (that is 40 mL/min/kg). Steady state volume distribution was found to be higher, and this could be one of the reasons of the tissue distribution. Last time of mean concentrations was 24 hours following intravenous bolus injection. Compound I-2 showed a 4.26-hour half-life in dog plasma. Oral administration of compound I-2 at 3 mg/kg to male Beagle dogs, demonstrated no lag phase in absorption and median time to reach peak concentration was 1 hour. Compound I-2 showed higher systemic availability. Oral bioavailability was found to be 83±22%. Overall, mean residence time and plasma half-life were comparable in plasma following intravenous and oral administration.

TABLE 8
Mean pharmacokinetic parameters of compound I-2 in male
Beagle dogs following single intravenous and oral gavage
administration at dose of 1 mg/kg and 3 mg/kg

| | IV (1 mg/kg) | Oral (3 mg/kg) |
|---|---|---|
| Tₘₐₓ (hours)^{a} | 0.08 (0.08-0.08) | 1.0 (0.5 to 1) |
| C₀/Cₘₐₓ (ng/mL) | 311.62 ± 98.3 | 372.74 ± 237.8 |
| AUCₗₐₛ (ng h/mL) | 528.66 ± 200.5 | 1295.08 ± 232.8 |
| AUC_{inf}(ng · h/mL) | 566.12 ± 191.4 | 1338.47 ± 242.1 |
| Cl (mL/min/kg) | 31.79 ± 10.7 | n/a |
| Vₛₛ (L/kg) | 9.35 ± 2.74 | n/a |
| Vd (L/kg) | 11.35 ± 2.93 | n/a |
| T_{1/2} (hour) | 4.26 ± 0.9 | ±1.27 |
| MRT (hour) | 3.82 ± 1.25 | 4.92 ± 0.97 |
| % F^{b} | n/a | 83 ± 22 |

C₀: Extrapolated concentration at time zero; C₀ reported for IV route; Cₘₐₓ reported for oral route
^{a}Reported median (min-max)
^{b}Nominal doses and AUC_{inf} of IV and oral were used to calculate bioavailability
% F^{b} is oral bioavailability

Compound I-2 attained good systemic exposure following oral administration. High steady state of volume of distribution was suggestive of portioning to tissues. Higher volume of distribution helps to sustain plasma concentrations until 24 hours post dose, which could be one of the reasons for moderate-high plasma half-life 4.26 and 5.81 hours in intravenous and oral, respectively. With its moderate clearance, compound I-2 showed very high oral bioavailability of 83% as compared to intravenous route.

Pharmacokinetic studies on mice and rat species have been completed and compound I-2 attained excellent systemic exposure with 28% in mice and 38% in rat oral bioavailability in its body weight dependent manner exposures.

### Biological Example 18

### In Vivo Tumor Efficacy Study

Anti-tumor efficacy of compound I-2 in 22RV1 induced neuroendocrine prostate cancer (NEPC) CDX male Nu/Nu (Balb/c background)

22Rv1 cells were cultured in RPMI supplemented with 10% FBS and 1% P/S, in a humidified incubator at 37° C. and 5% CO₂. Nu/nu Balb/c homozygous mice were procured through Jackson laboratory (Strain 002019). Mice were fed Teklad irradiated (sterilized) mouse diet and bedded with Teklad irradiated (sterilized) corncob bedding from Envigo (Indianapolis, IN). Mice were housed in Optimice carousel sterile quarters with filtered air supply in disposable cages from Animal Care Systems, Inc. (Centennial, CO). A 12-hour light/12-hour dark light cycle is observed, with animal handling only taking place during the light cycle.

22Rv1 Implantation: On the day of implantation, cells were trypsinized and allowed to detach from flasks. Trypsin was then neutralized with complete media and cells were spun at 300×g. Media was aspirated and cells were re-suspended in 50:50 Cultrex RPMI (no supplementation) at a concentration of 5×10⁷ cells/mL. A volume of 100 μL was injected into the right hind flank of each animal (a total of 5×10⁶ cells per mouse). Cells were checked for viability using trypan blue exclusion and found to be >98% viable.

Study Arms and Treatments: When mean tumor volume reached ˜225 mm³, mice were stratified and placed into (5) treatment groups as outlined below.

TABLE 9
22Rv1 Study Arms - Compound I-2 efficacy
study in Nu/Nu BALB/c mice

| | | | | | | Tumor |
|---|---|---|---|---|---|---|
| | | | | | | Volume/ |
| | | Test | Dose | Volume | | Body |
| Group | n | Article | (mg/kg) | (mL/kg) | Regimen | weight |
| 1 | 12 | Vehicle | 0 | 10 | QD × 21 | M/W/F |
| 2 | 12 | Compound | 30 | 10 | QD × 21 | M/W/F |
| 3 | 12 | I-2 | 50 | 10 | QD × 21 | M/W/F |
| 4 | 12 | | 65 | 10 | QD × 21 | M/W/F |
| 5 | 4 | | 60 | 10 | Single | M/W/F |
| | | | | | Dose | |

All mice were dosed orally, and the observation duration was 21 days

Treatments were administered by oral gavage (10 μL/gram adjusted volumes). Dosing solutions were made daily with molecular biology grade water as the vehicle (salt correction factor of 1.65 was applied the adjust for TFA salt)

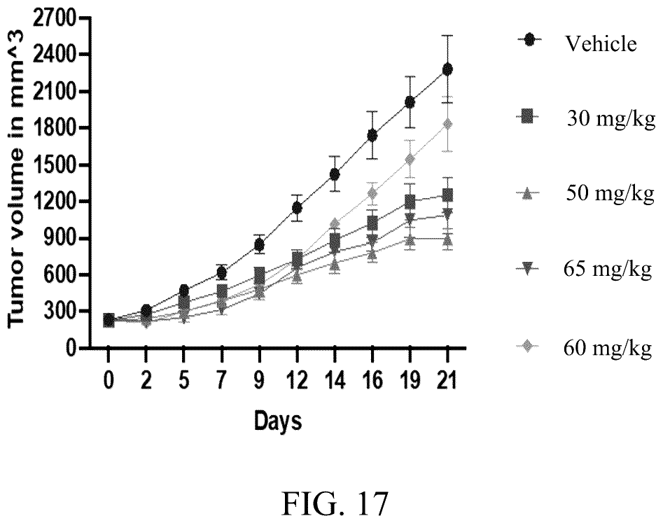

Animal Weights: Change in animal weight during a study is an additional indicator of tolerability. All animal weights are recorded (see, e.g., FIG. 16).

Relative body weights of groups in the study: 50 and 60 mg/kg groups, as well as the single dose 65 mg/kg group had mice drop from study starting on day 5. Tumor Growth Inhibition (TGI) data was analyzed against dose and tumor weights.

Tumor volumes: Tumor volumes were measured by digital caliper, and volume was calculated using the formula [Width²×Length]/2. All tumor volume measurements are recorded.

The 22Rv1 prostate cancer model is a model that was originally developed for growth of human prostate cancer in immunocompromised mice. In this example, tumors were grown subcutaneously for the efficacy study (4-arm study comparing vehicle to compound I-2 at 30 mg/kg, 50 mg/kg, and 65 mg/kg; dosed orally once daily, with an additional group of 4 mice dosed with a single dose at 60 mg/kg). Tolerability issues were noted with the >50 mg/kg dosing levels, causing some mice to be dropped from the study, and some mice to have dosing holidays for recovery. Compound I-2 appeared to inhibit tumor growth in a dose dependent manner, with tumor growth inhibition noted for all treatment groups.

Plasma, tumor, spleen, femur bone, heart, kidney, liver, lung, and brain were collected and processed 6 hours after a final dose for PK/PD studies.

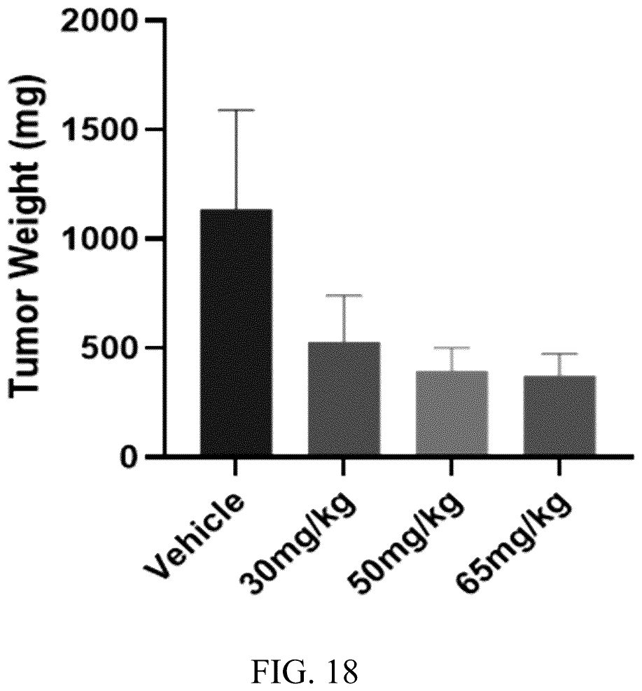

Results for average weight relative to day 0, average tumor volume changes over the 21-day study, and average tumor weight are shown in FIGS. 16-18, respectively.

### Biological Example 19

### In Vivo Tumor Efficacy Study 2

Anti-tumor efficacy of compound I-2 in LASCPC-01 induced neuroendocrine prostate cancer (NEPC) CDX male Nu/Nu (Balb/c background): Similar protocol to that of 22Rv1 study was implemented for LASCPC-01 study using doses of 30, 50, 65, and 80 mg/kg.

Change in animal weight during a study is an additional indicator of tolerability. All animal weights are recorded. Tumor volumes were measured by digital caliper and volume was calculated using the formula [Width²×Length]/2. All tumor volume measurements are recorded. The LASCPC-01 tumors exhibited robust growth (grew from 180 mm³ to 2700 mm³ in 19 days). Dose dependent tumor growth inhibition (TGI) was evident by observation.

50 and 80 mg/kg groups had mice drop from study starting on day 5. Vehicles had large tumors removed on day 19, therefore, day 21 does not reflect the average size of all mice in the group.

Compound I-2 appeared to inhibit tumor growth in a dose dependent manner, with a maximal growth inhibition of 117% at day 5 in the 80 mg/kg group. In the moderate dose group (50 mg/kg) maximal tumor growth inhibition of ˜100% was noted early in the study and remained >80% inhibition throughout the course of the study. Plasma, tumor, and bone were collected for PK/PD studies.

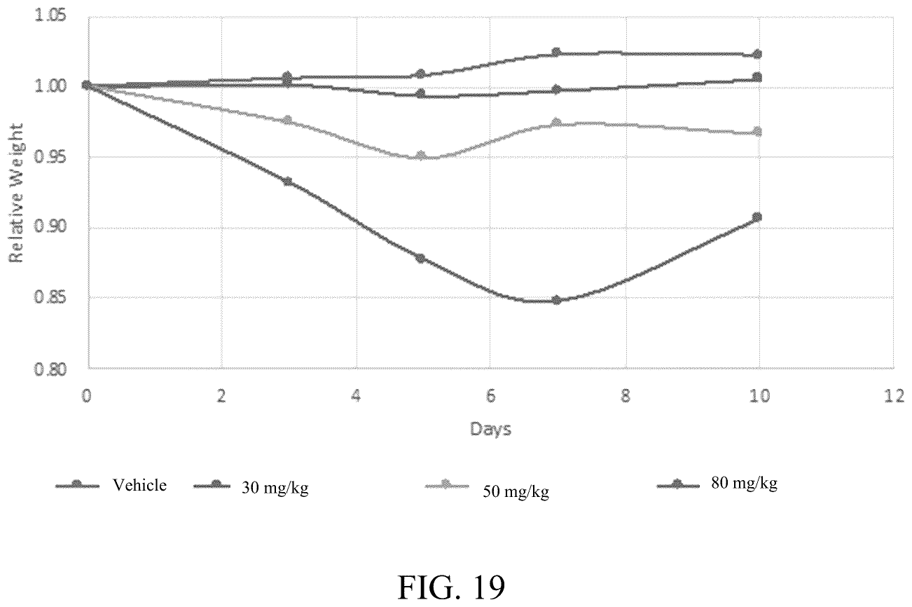

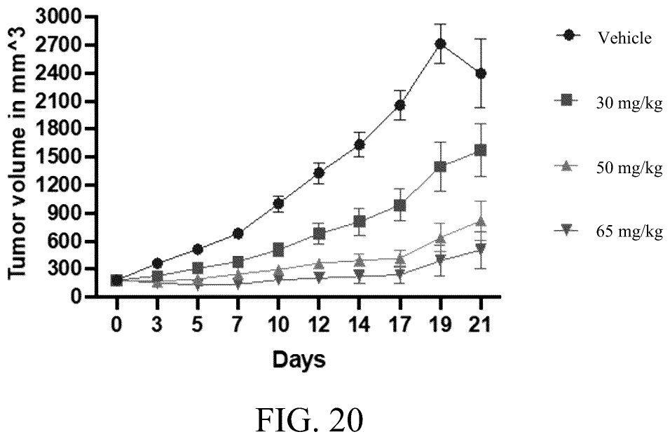

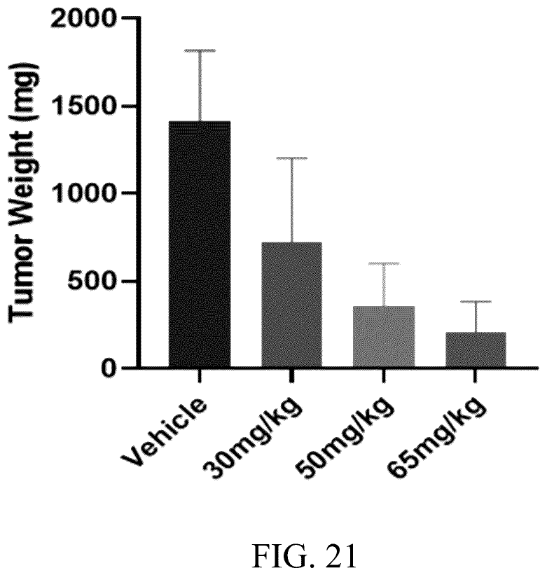

Results for average weight relative to day 0, average tumor volume changes over the 21-day study, and average tumor weight are shown in FIGS. 19-21, respectively.

### Biological Example 20

### In Vivo Tumor Efficacy Study 3

This example presents an evaluation and efficacy test for compound I-2 performed in a C4-2 human neuroendocrine prostate cancer xenograft model using male NOD·SCID mice.

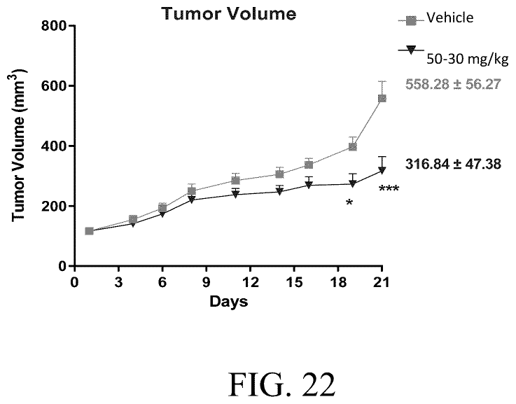

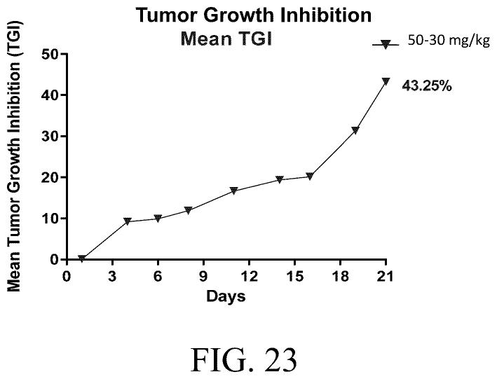

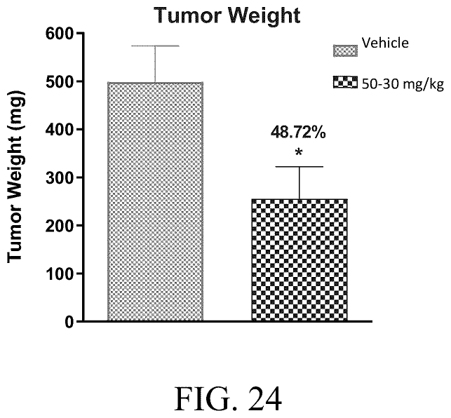

Body weight loss was observed when administering compound I-2 (50 mg/kg, p.o., daily) dose from day 1 to day 4. From day 8 initiated compound I-2 was administered at a dose of 30 mg/kg, bi-weekly. From day 13 onwards compound I-2 was dosed with 30 mg/kg, p.o., daily. Compound I-2 (50-30 mg/kg) showed decrease in tumor volume when compared to vehicle control group from day 8 to day 18. Compound I-2 at 50-30 mg/kg showed significant decrease tumor volume as compared to vehicle control group from days 18 to days 21 (see e.g., FIG. 22). Compound I-2 at doses 50-30 mg/kg showed increase in tumor growth inhibition when compared with vehicle control group from on days 8 to days 12 (see, e.g., FIG. 23). Compound I-2 at 50-30 mg/kg showed 43.25% tumor growth inhibition from vehicle control group on terminal day 21. Compound I-2 at 50-30 mg/kg showed significant decrease in tumor weight with 48.72% as compared to vehicle control group on day 21 (see, e.g., FIG. 24). Compound I-2 at 50-30 mg/kg showed significant body weight loss as compared to vehicle control group starting at day 4. Mortality was observed in one mouse for the compound I-2 treated group, possibly due to severe body weight loss to (i.e., ˜15%). 1.5 hours after the last administration blood was collected and processed, plasma and tumor samples were processed for bioanalytical LC-MS/MS and demonstrated promising PK/PD correlative efficacy.

Aspects of the embodiments can be modified, if necessary to employ concepts of the various patents, applications, and publications to provide yet further embodiments. These and other changes can be made to the embodiments considering the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

1. A compound having the following Structure (I): 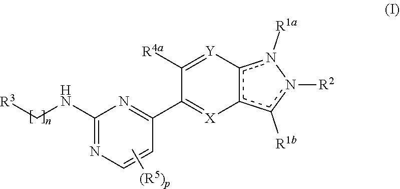
as a stereoisomer or salt thereof, wherein: [symbol]
represents a double or single bond such that all valences are satisfied and a heteroaryl ring is formed;
X is N or CR^{4b};
Y is N or CR^{4c};
R^{1a} is absent or C₁-C₆ alkyl;
R^{1b} is hydrogen, halo, hydroxy, C₁-C₆ alkyl, C₃-C₈ cycloalkyl, C₁-C₆ haloalkyl, C₁-C₆ alkoxy, or C₁-C₆ hydroxyalkyl;
R² is absent, hydrogen or optionally substituted: C₁-C₆ alkyl, C₃-C₈ cycloalkyl, C₁-C₆ haloalkyl, or C₁-C₆ hydroxyalkyl;
R³ is C₃-C₅ cycloalkyl, C₆-C₈ bridged cycloalkyl, or 3-10 membered heterocyclyl, optionally substituted with hydroxyl, amino, cyano, halo, C₁-C₆ alkyl, C₃-C₈ cycloalkyl, C₁-C₆ haloalkyl, C₁-C₆ alkoxy, C₁-C₆ haloalkoxy, or —N(R^{3c})R^{3a}, or
R³ is C₃-C₈ cycloalkyl optionally substituted with hydroxyl, cyano, halo, C₁-C₆ alkyl, C₃-C₈ cycloalkyl, C₁-C₆ haloalkyl, C₁-C₆ alkoxy, C₁-C₆ haloalkoxy, or —N(R^{3c})R^{3b};
R^{3a} is optionally substituted 3-10 membered heterocyclyl or optionally substituted 3-10 membered heteroaryl;
R^{3b} is optionally substituted 3-10 membered N-heterocyclyl or optionally substituted 3-10 membered heteroaryl;
R^{3c} is hydrogen or optionally substituted C₁-C₆ alkyl;
R^{4a}, R^{4b}, and R^{4c} are each independently hydrogen, halo or optionally substituted: C₁-C₆ alkyl, C₃-C₈ cycloalkyl, C₁-C₆ haloalkyl, C₁-C₆ alkoxy, or C₁-C₆ haloalkoxy;
each occurrence of R⁵ is independently halo or optionally substituted C₁-C₆ haloalkyl;
n is 0, 1, 2, 3, or 4; and
p is 0, 1, or 2.

2. The compound of claim 1, wherein the compound has one of the following structures (Ia) or (Ib):

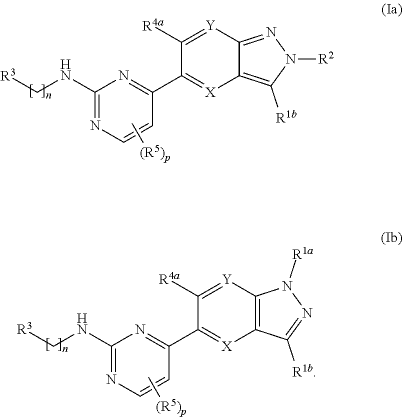

3. The compound of claim 1, wherein R^{1a} is absent or methyl.

4-6. (canceled)

7. The compound of claim 1, wherein R^{1b} is hydrogen, cyclopropyl, cyclobutyl, isopropyl, or methyl.

8-10. (canceled)

11. The compound of claim 1, wherein R² is —CH₃.

12-14. (canceled)

15. The compound of claim 1, wherein both of R^{1b} and R² are hydrogen.

16. The compound of claim 1, wherein n is 0 or 1.

17. (canceled)

18. The compound of claim 1, wherein R³ is C₃-C₅ cycloalkyl, C₆-C₇ bridged cycloalkyl, or 3-10 membered heterocyclyl optionally substituted with hydroxyl, amino, cyano, halo, C₁-C₆ alkyl, C₃-C₈ cycloalkyl, C₁-C₆ haloalkyl, C₁-C₆ alkoxy, C₁-C₆ haloalkoxy, or —N(R^{3c})R^{3a}.

19-24. (canceled)

25. The compound of claim 1, wherein R³ has one of the following structures:

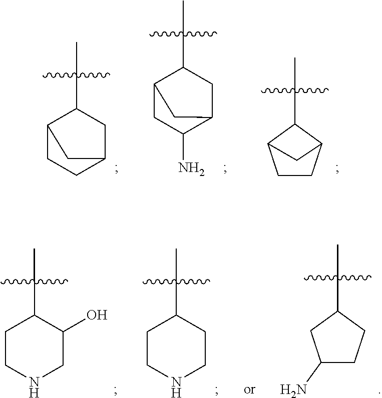 [SMILES: *C1CC2CCC1C2 ; *C1CC2CC1CC2N ; *C1C2CCC1C2 ; *C1CCNCC1O ; *C1CCNCC1 ; *C1CCC(N)C1]

26. (canceled)

27. The compound of claim 1, wherein R^{4a} is hydrogen.

28. The compound of claim 1, wherein X is N, CH, or CF.

29. (canceled)

30. The compound of claim 1, wherein Y is N, CH, or CF.

31-32. (canceled)

33. The compound of claim 1, wherein p is 1 and R⁵ is chloro.

34. The compound of claim 1, wherein p is 0.

35. The compound of claim 1, wherein the compound has one of the following structures: 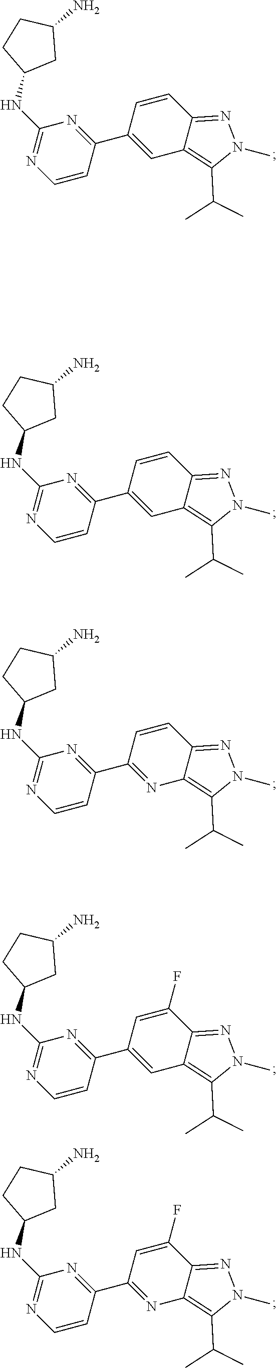 [SMILES: CC(C)c1c2cc(-c3ccnc(N[C@@H]4CC[C@H](N)C4)n3)ccc2nn1C ; CC(C)c1c2cc(-c3ccnc(N[C@H]4CC[C@H](N)C4)n3)ccc2nn1C ; CC(C)c1c2nc(-c3ccnc(N[C@H]4CC[C@H](N)C4)n3)ccc2nn1C ; CC(C)c1c2cc(-c3ccnc(N[C@H]4CC[C@H](N)C4)n3)cc(F)c2nn1C ; CC(C)c1c2nc(-c3ccnc(N[C@H]4CC[C@H](N)C4)n3)cc(F)c2nn1C] 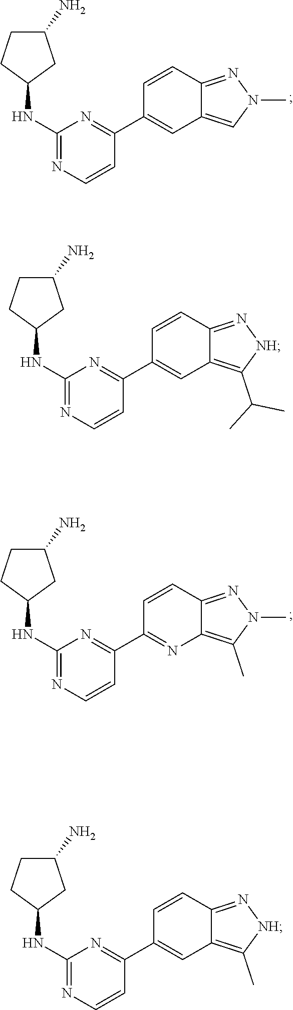 [SMILES: Cn1cc2cc(-c3ccnc(N[C@H]4CC[C@H](N)C4)n3)ccc2n1 ; CC(C)c1[nH]nc2ccc(-c3ccnc(N[C@H]4CC[C@H](N)C4)n3)cc12 ; Cc1c2nc(-c3ccnc(N[C@H]4CC[C@H](N)C4)n3)ccc2nn1C ; Cc1[nH]nc2ccc(-c3ccnc(N[C@H]4CC[C@H](N)C4)n3)cc12] 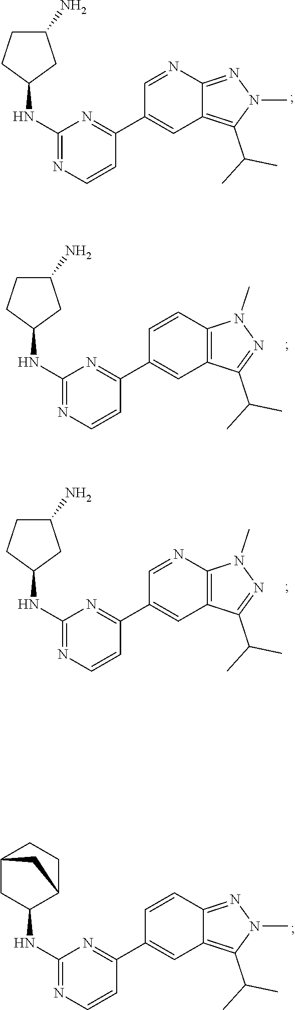 [SMILES: CC(C)c1c2cc(-c3ccnc(N[C@H]4CC[C@H](N)C4)n3)cnc2nn1C ; CC(C)c1nn(C)c2ccc(-c3ccnc(N[C@H]4CC[C@H](N)C4)n3)cc12 ; CC(C)c1nn(C)c2ncc(-c3ccnc(N[C@H]4CC[C@H](N)C4)n3)cc12 ; CC(C)c1c2cc(-c3ccnc(N[C@H]4C[C@@H]5CC[C@H]4C5)n3)ccc2nn1C] 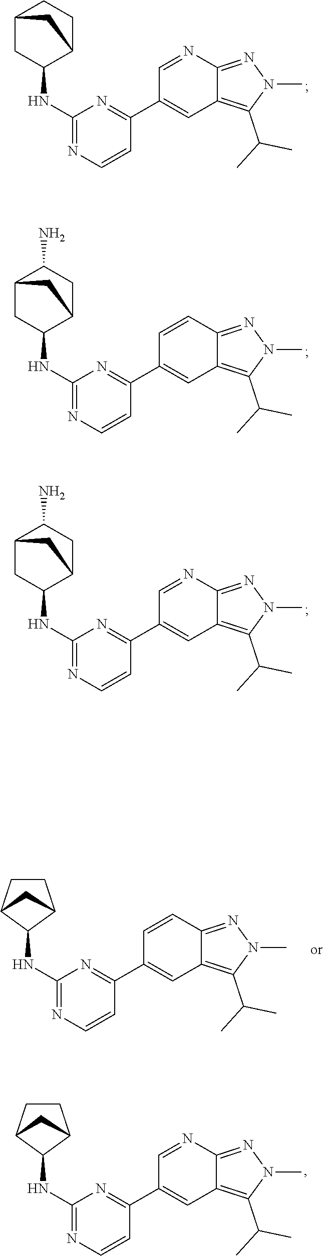 [SMILES: CC(C)c1c2cc(-c3ccnc(N[C@H]4C[C@@H]5CC[C@H]4C5)n3)cnc2nn1C ; CC(C)c1c2cc(-c3ccnc(N[C@H]4C[C@H]5C[C@@H]4C[C@H]5N)n3)ccc2nn1C ; CC(C)c1c2cc(-c3ccnc(N[C@H]4C[C@H]5C[C@@H]4C[C@H]5N)n3)cnc2nn1C ; CC(C)c1c2cc(-c3ccnc(N[C@H]4[C@@H]5CC[C@H]4C5)n3)ccc2nn1C ; CC(C)c1c2cc(-c3ccnc(N[C@H]4[C@@H]5CC[C@H]4C5)n3)cnc2nn1C]
or a stereoisomer or salt thereof.

36. The compound of claim 1, wherein the compound is a free base form.

37. The compound of claim 1, wherein the compound is a pharmaceutically acceptable salt.

38. The compound of claim 1, wherein the compound is a trifluoroacetic acid salt, a hydrochloric acid salt, or a formic acid salt.

39. A pharmaceutical composition comprising a compound or salt of claim 1 and a pharmaceutically acceptable carrier.

40. A method treating a disease or disorder, the method comprising administering an effective amount of the compound claim 1 to a subject in need thereof.

41. The method of claim 40, wherein the disease or disorder is a kinase-expressing cancer, an autoimmune disease, or an inflammatory disease.

42. The method of claim 41, wherein the cancer is bladder cancer, prostate cancer, or acute myeloid leukemia.

43-46. (canceled)
